SEQUENCE LISTING (23 sequences)

SEQ ID NO: 1 - DNA, 8 bp, Artificial Sequence
  Synthetic Sequence
cataactg

SEQ ID NO: 2 - DNA, 8 bp, Artificial Sequence
  Synthetic Sequence
aataactg

SEQ ID NO: 3 - DNA, 8 bp, Artificial Sequence
  Synthetic Sequence
caatattg

SEQ ID NO: 4 - DNA, 8 bp, Artificial Sequence
  Synthetic Sequence
ctacactt

SEQ ID NO: 5 - DNA, 8 bp, Artificial Sequence
  Synthetic Sequence
aataactg

SEQ ID NO: 6 - DNA, 8 bp, Artificial Sequence
  Synthetic Sequence
cataactg

SEQ ID NO: 7 - DNA, 8 bp, Artificial Sequence
  Synthetic Sequence
gctaactg

SEQ ID NO: 8 - DNA, 8 bp, Artificial Sequence
  Synthetic Sequence
ctacattg

SEQ ID NO: 9 - DNA, 8 bp, Artificial Sequence
  Synthetic Sequence
gcatattg

SEQ ID NO: 10 - DNA, 4 bp, Artificial Sequence
  Synthetic Sequence
aata

SEQ ID NO: 11 - DNA, 4 bp, Artificial Sequence
  Synthetic Sequence
cata

SEQ ID NO: 12 - DNA, 4 bp, Artificial Sequence
  Synthetic Sequence
gcta

SEQ ID NO: 13 - DNA, 4 bp, Artificial Sequence
  Synthetic Sequence
gcta

SEQ ID NO: 14 - DNA, 4 bp, Artificial Sequence
  Synthetic Sequence
ctac

SEQ ID NO: 15 - DNA, 4 bp, Artificial Sequence
  Synthetic Sequence
gcat

SEQ ID NO: 16 - DNA, 8 bp, Artificial Sequence
  Synthetic Sequence
caatattg

SEQ ID NO: 17 - DNA, 8 bp, Artificial Sequence
  Synthetic Sequence
aataactg

SEQ ID NO: 18 - DNA, 10 bp, Artificial Sequence
  Synthetic Sequence
agactgaccg

SEQ ID NO: 19 - DNA, 10 bp, Artificial Sequence
  Synthetic Sequence
agactgacgt

SEQ ID NO: 20 - DNA, 10 bp, Artificial Sequence
  Synthetic Sequence
ctggactctc

SEQ ID NO: 21 - DNA, 10 bp, Artificial Sequence
  Synthetic Sequence
ctactgacgg

SEQ ID NO: 22 - DNA, 10 bp, Artificial Sequence
  Synthetic Sequence
ctaccgacgt

SEQ ID NO: 23 - DNA, 10 bp, Artificial Sequence
  Synthetic Sequence
agggaatctc

(19) United States
(12) Patent Application Publication
(10) Pub. No.: US 2023/0265499 A1
(43) Pub. Date: Aug. 24, 2023

(54) In Situ Library Preparation for Sequencing

(71) Applicant: Factorial Diagnostics, Inc., San Carlos, CA (US)

(72) Inventors: John Daniel Wells, San Francisco, CA (US); Katie Leigh Zobeck, Sunnyvale, CA (US)

(21) Appl. No.: 18/021,176

(22) PCT Filed: Aug. 13, 2021
(86) PCT No.: PCT/US2021/046025

§ 371 Date: Feb. 14, 2023

Related U.S. Application Data
(60) Provisional application No. 63/066,071, filed on Aug. 14, 2020.
(60) Provisional application No. 63/134,079, filed on Jan. 5, 2021.

(51) Int. Cl. C12Q 1/6869; C12N 15/10; C12Q 1/6841

(52) U.S. Cl. CPC C12Q 1/6869; C12N 15/1065; C12Q 1/6841; C12Q 1/6804

(57) ABSTRACT

Aspects of the present disclosure relate generally to methods, compositions, and kits for preparing a ligation-based or amplicon-based library in situ for sequencing.

### 1. CROSS REFERENCE TO RELATED APPLICATIONS

This application claims the benefit of U.S. Provisional Application Ser. No. 63/066,071 filed Aug. 14, 2020, and U.S. Provisional Application Ser. No. 63/134,079, filed Jan. 5, 2021, which are hereby incorporated by reference in their entireties.

### 2. INTRODUCTION

Current target enrichment methods for analyzing genomic alterations include multiplexed PCR and hybrid capture performed on either purified nucleic acid or crude cell extracts. These methods are excellent for identifying homozygous and heterozygous mutations in the genome, and RNA expression levels in populations of cells. However, these methods typically use purified genomic DNA, from a large number of cells, so information regarding population heterogeneity is lost in the analysis and the methods have had problems scaling to small cell populations. Furthermore, spatial information of single cells in the tissue is often lost during the isolation step and thus single-cell sequencing data typically do not show how cells are organized to implement the concerted function within a tissue of interest.

Conventional library preparation techniques are performed outside of the natural microenvironment of the cell after cell lysis, and have not been successfully attempted inside the cell due to observed inhibitory effects seen with many methods for making crude lysates. Due to the cell lysis before library preparation, phenotypic and occasionally genotypic information of subpopulations is lost, especially if that population is rare. Causing either a large upfront cost of obtaining enough cells for sub-sorting before genomic isolation or performing DNA sequencing at a very high depth to obtain enough reads to identify a rare variant, both options are costly and inefficient.

New techniques are needed to resolve the heterogeneity of, for example, cancer cells and tumor-infiltrating immune cells, may provide new insights into the regulatory mechanisms within tumors and new drug targets to modulate tumor progression. Single cell sequencing is rising to this challenge, but existing methods are slow, expensive, low throughput and can only identify a small subset of target regions.

Therefore, there is a need for cost effective and sensitive multi-omics techniques for measuring disease-associated genetic alterations (e.g., reveals genetic, epigenetic, and transcriptomic heterogeneity) associated with heterogenous cell populations. There is also a need to preserve phenotypic information during library preparation and whole genomic sequencing or Next Generation Sequencing (NGS) to increase detection sensitivity of disease-associated genetic alterations in a heterogenous cell population.

### 3. SUMMARY

Aspects of the present disclosure relate generally to methods, compositions, and kits for preparing a ligation- or amplicon-based library in situ for sequencing.

The present inventors developed in situ amplicon-based and in situ ligation-based library preparation methods to prepare sequencing libraries (e.g., NGS sequencing libraries) for a multitude of individual cells within one reaction. In one example, the method utilizes the cell membrane to contain the genetic information into individual cell reactions within a single reaction, as opposed to the current NGS library preparation methods that require physical separation of populations or cells, so that they can be lysed before library preparation. By keeping the cells intact, phenotypic markers, including RNA or protein expression can be used to select for samples of interest. This allows for smaller populations (1-100 cells) to be analyzed without the need to develop a library preparation protocol for tiny amounts of cells or DNA. For example, by performing library preparation in situ, using the methods described herein, the present inventors found that subpopulations of 10 cells or less can be enriched with high yield containing a target DNA or RNA of interest, and the cell environment can be preserved. Performing library preparation inside cells in situ allows for enriching cell populations, such as in a cell suspension, of interest where the number of cells is low such as for rare cell populations. By performing library preparation inside cells in situ, the present inventors were able to enrich and sequence cell populations of interest with high yield.

Detailed understanding of tumor ecosystems at single-cell resolution has been limited for technological reasons. Conventional genomic, transcriptomic, and epigenomic sequencing protocols require tens-hundreds of nanograms of purified input material, and genomic DNA purification on its own required a large amount of input material. Thus to isolate enough material cancer-related genomic studies have been limited to using a large section of the tumor in the DNA isolation, yielding in bulk tumor sequencing, which masks the effects of genetic intratumor heterogeneity. Additionally, conventional techniques of bulk tumor sequencing fail to provide phenotypic insight of tumor heterogeneity, requiring multiple disparate tests to be performed on a tumor sample. Phenotypic heterogeneity of tumors, especially the percent of cancer cells and tumor-infiltrating immune cells can provide insight into regulatory mechanisms within tumors and new drug targets to modulate tumor progression.

Aspects of the present disclosure relate generally to methods, compositions, and kits for determining the phenotypic heterogeneity between cell populations in a sample and identifying disease-associated genetic alterations of distinct cell populations within the sample. Aspects of the present disclosure also include a computer readable-medium and a processor to carry out the steps of the methods or instructions of the kits described herein.

Aspects of the present disclosure include methods for determining heterogeneity of mixed cell populations and/or subcellular populations. Aspects of the present disclosure also include methods for determining heterogeneity of one or more cell populations within a tumor. Aspects of the present disclosure also include methods for labeling individual intact cells within one or more cell populations.

Aspects of the present disclosure include methods for determining the heterogeneity of a tumor, the method comprising: (a) providing a sample comprising a heterogenous cell population; (b) contacting one or more cell populations with a fragmentation buffer and a fragmentation enzyme to form a mixture; (c) performing an enzymatic fragmentation reaction on the mixture to form fragmented DNA or RNA within the one or more cell populations; (d) contacting the one or more cell populations comprising fragmented DNA or RNA with a set of indexing nucleotide sequences; (e) ligating the fragmented DNA or RNA to the indexing nucleotide sequences to produce an indexed library; (f) performing hybridization capture on the indexed library to produce an enriched indexed library; and (g) analyzing the enriched indexed library to determine the presence or absence of disease-associated genetic alterations within the cell populations.

An aspect of the present disclosure provides a method for preparing a ligation-based library in situ for sequencing, the method comprising: (a) providing a sample comprising a heterogenous cell/nuclei population having a plurality of phenotypes; (b) performing, in each cell/nuclei of the heterogenous cell/nuclei population, an enzymatic fragmentation reaction to form DNA fragments within the heterogenous cell/nuclei population; (c) ligating, in each cell/nuclei, the DNA fragments to adapter sequences in situ to create a ligated library comprising ligated DNA fragments; (d) sorting the cell/nuclei of the heterogenous cell/nuclei populations into subpopulations by phenotypes to determine target cells/nuclei and non-target cells/nuclei; (e) lysing each of the target cells/nuclei to collect the ligated DNA fragments; (f) purifying the ligated DNA fragments from the target cells/nuclei; and (g) sequencing the ligated DNA fragments from the target cells/nuclei.

In some embodiments, after step (c), but before step (e), the method further comprises amplifying the ligated DNA fragments to form amplicon products. In some embodiments, after step (e), but before step (g), the method further comprises amplifying the ligated DNA fragments to form amplicon products. In some embodiments, after step (e) but before step (g) the method comprises ligating the ligated DNA fragments with barcode adapter sequences.

In some embodiments, the method comprises, before step (a), adding primary antibodies to the sample, and wherein the method comprises, before step (d), adding detectable secondary antibodies or other detectable molecules to the sample. In some embodiments, the method comprises, before step (d), adding primary antibodies, followed by a detectable secondary antibody or other detectable molecule to the sample In some embodiments, the method comprises, before step (d), adding a detectable primary antibody to the sample.

In some embodiments, before step (c), performing an end-repair and A-tailing reaction on the one or more DNA fragments. In some embodiments, the end-repair and A-tailing reaction and the enzymatic fragmentation reaction is a single reaction.

In some embodiments, multiple PCR reactions are performed between steps (c) and (g).

In some embodiments, ligating the DNA fragments to the adapter sequences comprises running the DNA fragments and adapter sequences in a thermocycler at a temperature and duration sufficient to ligate the DNA fragmented to the adapter sequences. In some embodiments, the adapter sequences comprise Y-adapter nucleotide sequences, hairpin nucleotide sequences, or duplex nucleotide sequences.

In some embodiments, the contacting in step (e) comprises amplifying the ligated library to produce a barcoded indexed library. In some embodiments, the barcode adapter sequences comprise a set of forward and/or reverse barcoding adapters. In some embodiments, ligating the ligated DNA fragments with forward and/or reverse barcode adapters produce a barcoded indexed library.

In some embodiments, the method further comprises, before step (h), performing hybridization capture on the ligated DNA fragments. In some embodiments, the method further comprises, before step (h), performing hybridization capture on the barcoded indexed library.

In some embodiments, the ligating the barcode adapter sequences occurs before sorting in step (d), after step (d) but before step (e), or after step (e). In some embodiments, before step (b), the method comprises fixing and/or permeabilizing the heterogenous cell population.

In some embodiments, said sequencing comprises next generation sequencing. In some embodiments, each population of target cells comprises 3-10 cells. In some embodiments, the sample is a cell suspension generated from a tissue sample or a cell suspension generated from a liquid biopsy. In some embodiments, the sample is a Formalin-Fixed Paraffin-Embedded (FFPE) tissue sample or a cryopreserved tissue sample.

Further aspects of the present disclosure provide a method for preparing an amplicon-based library in situ for sequencing, the method comprising: (a) providing a sample comprising a heterogenous cell/nuclei population having a plurality of cell/nuclei phenotypes; (b) amplifying, in each cell/nuclei within the heterogenous population, DNA with a primer pool set to produce a first set of amplicon products for each cell/nuclei; (c) sorting the cell/nuclei phenotypes of the heterogenous cell populations into subpopulations by phenotypes to determine target cells and non-target cells; (d) lysing each of the target cells to isolate DNA fragments from the first set of amplicon products; (e) purifying the first set of amplicon products from the target cells; and (f) sequencing the first set of amplicon products from the target cells.

In some embodiments, before step (b), the method comprises fixing and/or permeabilizing the heterogenous cell population. In some embodiments, before step (d), wherein the method further comprises amplifying the first set of amplicon products with adapter sequences to produce a second set of amplicon products. In some embodiments, the method further comprises, after step (c) or (d), contacting the first set of amplicon products with barcoding sequences. In some embodiments, said barcoding sequences comprise a set of forward and/or reverse barcoding primers, and wherein the method comprises amplifying the first set of amplicon products with the set of forward and/or reverse barcoding primers to produce a barcoded indexed library comprising barcoded amplicon products.

In some embodiments, said barcoding sequences comprise a set of forward and/or reverse barcoding adapters, and wherein the method comprises ligating the set of forward and/or reverse barcode adapters to produce a barcoded indexed library comprising barcoded amplicon products.

In some embodiments, before step (b), the method comprises fixing and/or permeabilizing the heterogenous cell population. In some embodiments, the primer pool set comprises primers that hybridize to a target region of a target sequence of the DNA within the heterogenous cell population. In some embodiments, the primer pool set further comprises indexing primers.

In some embodiments, the sample is a cell suspension generated from a tissue sample or a cell suspension generated from a liquid biopsy. In some embodiments, the sample is a Formalin-Fixed Paraffin-Embedded (FFPE) tissue sample or a cryopreserved tissue sample. In some embodiments, said sequencing comprises next generation sequencing.

In some embodiments, said contacting occurs before or after sorting in step (c). In some embodiments, said contacting occurs after lysing in step (d). In some embodiments, each population of target cells comprises 3-10 cells. In some embodiments, multiple PCR reactions are performed between steps (c) and (0.

Further aspects of the present disclosure provide a method for preparing a ligation-based library in situ for sequencing, the method comprising: (a) providing a sample comprising a cell/nucleic population; (b) performing, in each cell of the cell/nuclei population, an enzymatic fragmentation reaction to form DNA fragments within the cell/nuclei population; (c) ligating, in each cell, the DNA fragments to adapter sequences in situ to create a ligated library comprising ligated DNA fragments; (d) lysing each of the cells to collect the ligated DNA fragments; (e) purifying the ligated DNA fragments (e.g., derived from the cells); and (f) sequencing the ligated DNA fragments (e.g., derived from the cells).

In some embodiments, the method comprises, after step (c), sorting the cell/nuclei population into subpopulations irrespective of phenotype. In some embodiments, the method comprises, after step (c), sorting the cell/nuclei population into subpopulations by phenotypes to determine target cells/nuclei and non-target cells/nuclei. In some embodiments, the method comprises, after step (c), but before step (d), the method further comprises amplifying the ligated DNA fragments to form amplicon products. In some embodiments, after step (d), but before step (f), the method further comprises amplifying the ligated DNA fragments with amplification primers to form amplicon products. In some embodiments, after step (d) but before step (f) the method comprises ligating the ligated DNA fragments with barcode adapter sequences.

In some embodiments, the method comprises, before step (a), adding primary antibodies to the sample, and wherein the method comprises, before step (d), adding detectable secondary antibodies or other detectable molecules to the sample. In some embodiments, the method comprises, before step (d), adding primary antibodies, followed by a detectable secondary antibody or other detectable molecule to the sample In some embodiments, the method comprises, before step (d), adding a detectable primary antibody to the sample.

In some embodiments, before step (c), performing an end-repair and A-tailing reaction on the one or more DNA fragments. In some embodiments, the end-repair and A-tailing reaction and the enzymatic fragmentation reaction is a single reaction.

In some embodiments, multiple PCR reactions are performed between steps (c) and (f).

In some embodiments, ligating the DNA fragments to the adapter sequences comprises running the DNA fragments and adapter sequences in a thermocycler at a temperature and duration sufficient to ligate the DNA fragmented to the adapter sequences. In some embodiments, the adapter sequences comprise Y-adapter nucleotide sequences, hairpin nucleotide sequences, or duplex nucleotide sequences. In some embodiments, the method comprises, after step (d), contacting the ligated DNA fragments with a set of forward and/or reverse barcoding primers, and amplifying the ligated DNA fragments to produce a barcoded indexed library. In some embodiments, the barcode adapter sequences comprise a set of forward and/or reverse barcoding adapter sequences.

In some embodiments, ligating the ligated DNA fragments with forward and/or reverse barcode adapter sequences produce a barcoded indexed library.

In some embodiments, the method further comprises, before step (f), performing hybridization capture on the ligated DNA fragments. In some embodiments, the method further comprises, before step (f), performing hybridization capture on the barcoded indexed library.

In some embodiments, said ligating the forward and/or reverse barcode adapter sequences occurs before sorting, after sorting but before purifying in step (e), or after purifying in step (e). In some embodiments, before step (b), the method comprises fixing and/or permeabilizing the heterogenous cell population. In some embodiments, said sequencing comprises next generation sequencing.

In some embodiments, the cell population comprises target cells having 3-10 cells. In some embodiments, the sample is a cell suspension generated from a tissue sample or a cell suspension generated from a liquid biopsy. In some embodiments, the sample is a Formalin-Fixed Paraffin-Embedded (FFPE) tissue sample or a cryopreserved tissue sample.

Further aspects of the present disclosure provide a method for preparing an amplicon-based library in situ for sequencing, the method comprising: (a) providing a sample comprising a cell/nuclei population; (b) amplifying, in each cell within the cell/nuclei population, DNA with a primer pool set to produce a first set of amplicon products for each cell; (c) lysing each of the cells to isolate DNA fragments within the first set of amplicon products; (d) purifying the DNA fragments; and (e) sequencing the DNA fragments.

In some embodiments, the method comprises, after step (b), sorting the cell/nuclei population into subpopulations by phenotypes to determine target cells/nuclei and non-target cells/nuclei.

In some embodiments, before step (c), wherein the method further comprises amplifying the first set of amplicon products with adapter sequences to produce a second set of amplicon products. In some embodiments, the method further comprises, after step (b) or (c), contacting the first set of amplicon products with sample barcoding sequences.

In some embodiments, said sample barcoding sequences comprise a set of forward and/or reverse barcoding primers, and wherein the method comprises amplifying the first set of amplicon products with the set of forward and/or reverse barcoding primers to produce a barcoded indexed library comprising barcoded amplicon products.

In some embodiments, said barcoding sequences comprise a set of forward and/or reverse barcoding adapters, and wherein the method comprises ligating the set of forward and/or reverse barcode adapters to produce a barcoded indexed library comprising barcoded amplicon products. In some embodiments, before step (b), the method comprises fixing and/or permeabilizing the/nuclei population. In some embodiments, the primer pool set comprises primers that hybridize to a target region of a target sequence of the DNA within the/nuclei population. In some embodiments, the primer pool set further comprises indexing primers.

In some embodiments, the sample is a cell suspension generated from a tissue sample or a cell suspension generated from a liquid biopsy. In some embodiments, the sample is a Formalin-Fixed Paraffin-Embedded (FFPE) tissue sample or a cryopreserved tissue sample. In some embodiments, said sequencing comprises next generation sequencing.

In some embodiments, the method further comprises, after step (b), sorting the cell/nucleic population into subpopulations by phenotypes to determine target cells/nucleic and non-target cells/nuclei. In some embodiments, said contacting occurs after lysing in step (c). In some embodiments, the cell population comprises target cells having 3-10 cells. In some embodiments, multiple PCR reactions are performed between steps (b) and (e). In some embodiments, before step (b), the method comprises fixing and/or permeabilizing the heterogenous cell population.

Aspects of the present disclosure further provide a kit for amplicon-based library preparation in situ, the kit comprising: a cell preservation agent capable of preserving a cell/nuclei population, the cell preservation agent selected from: a fixative, a permeabilizer, or a fixative and a permeabilizer; a primer pool set capable of amplifying a target sequence region of DNA within one or more cells of the cell/nuclei population; a polymerase chain reaction (PCR) Enzyme Master Mix comprising one or more of: an enzyme, a buffer, or an enzyme and a buffer; a cell lysis buffer; in an amount sufficient to prepare an amplicon-based library in situ for sequencing; and instructions for carrying out the amplicon-based library preparation in situ, the instructions providing the following steps: amplifying the target sequence region of DNA in the cell/nuclei population to produce a first set of amplicon products for each cell; lysing each of the cells to isolate DNA fragments having the target sequence region within the first set of amplicon products; purifying the DNA fragments; and sequencing the DNA fragments.

In some embodiments, the kit further comprises protease K for the lysing step. In some embodiments, the kit further comprises barcoding primers, and a second PCR Enzyme master mix comprising one or more of: an enzyme, a buffer, or an enzyme and a buffer.

Aspects of the present disclosure provide a kit for ligation-based library preparation in situ, the kit comprising: a cell preservation agent capable of preserving a cell/nuclei population, the cell preservation agent selected from: a fixative, a permeabilizer, or a fixative and a permeabilizer; a fragmentation enzyme and buffer for performing an enzymatic fragmentation reaction to form DNA fragments within the cell/nuclei population; optionally an End repair and A tail (ERA) master mix and buffer for performing an end-repair and A-tailing reaction on the one or more DNA fragments; a ligation enzyme and buffer; adapter sequences, wherein the ligation enzyme and buffer, and adapter sequences are capable of ligating, in each cell, the DNA fragments to the adapter sequences in situ to create a ligated library comprising ligated DNA fragments; amplification primers for amplifying the ligated DNA fragments to form amplicon products; a polymerase chain reaction (PCR) enzyme master mix comprising one or more of: an enzyme, a buffer, or an enzyme and a buffer; a cell lysis buffer; in an amount sufficient to prepare a ligation-based library in situ for sequencing; and instructions for carrying out the ligation-based library preparation in situ, the instructions providing the following steps: performing, in each cell of the cell/nuclei population, an enzymatic fragmentation reaction to form DNA fragments within the cell/nuclei population; ligating, in each cell, the DNA fragments to adapter sequences in situ to create the ligated library comprising ligated DNA fragments; lysing each of the cells to collect the ligated DNA fragments; purifying the ligated DNA fragments; and sequencing the ligated DNA fragments.

In some embodiments, the amplification primers comprise barcoding primers, sequencing primers, or a combination thereof. In some embodiments, the kit further comprises protease K for the lysing step. In some embodiments, the kit further comprises barcoding primers, and a second PCR Enzyme master mix comprising one or more of: an enzyme, a buffer, or an enzyme and a buffer.

### 4. BRIEF DESCRIPTION OF THE DRAWINGS

FIG. 1 provides an overview of the workflow of an aspect of the present disclosure in identifying phenotypic labels after preparing the library, sorting the cells by phenotype, and performing NGS.

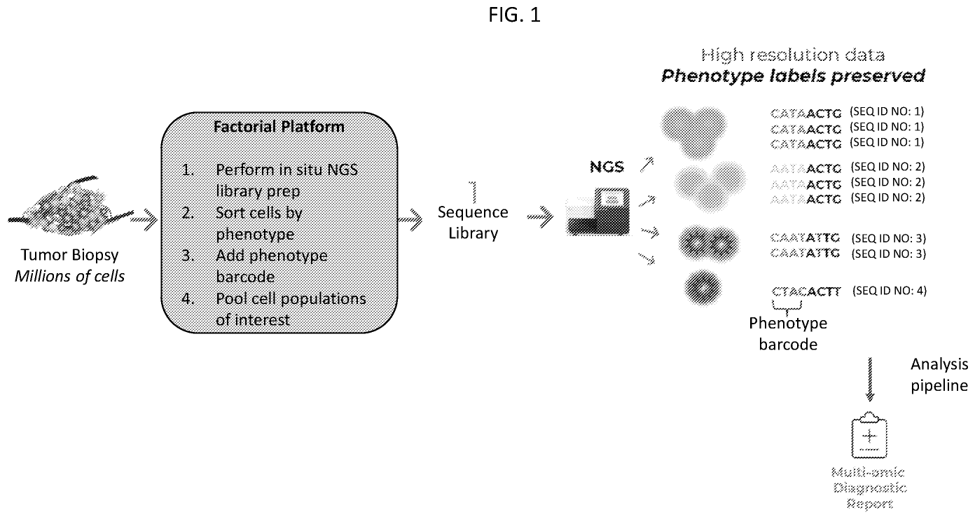

FIG. 2 shows an aspect of the present disclosure, of analyzing a plurality of phenotypically distinct cell populations.

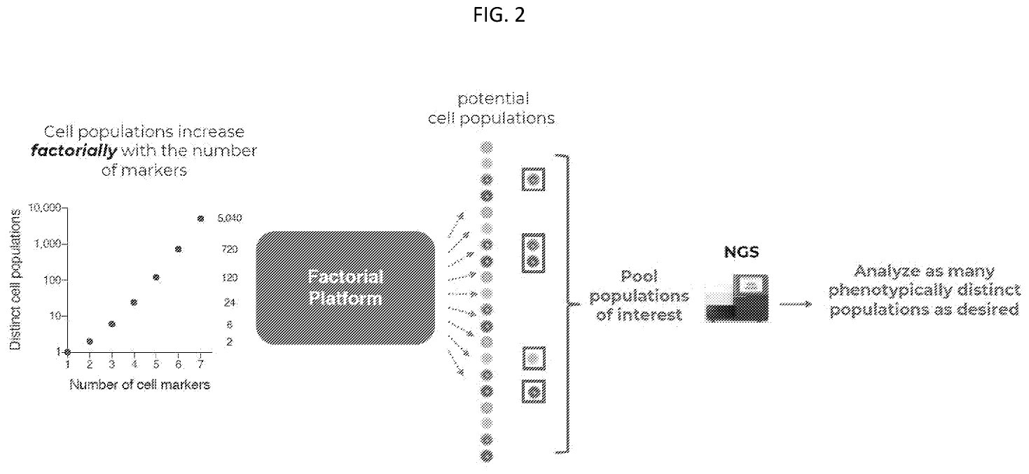

FIG. 3 provides a detailed workflow of an aspect of the present disclosure.

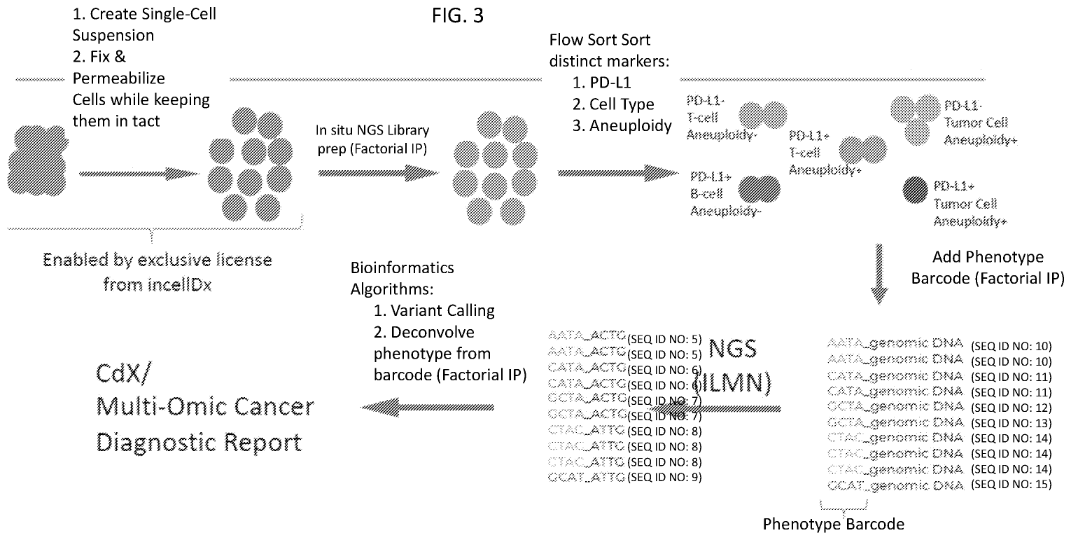

FIG. 4 provides a detailed workflow of an aspect of the present disclosure.

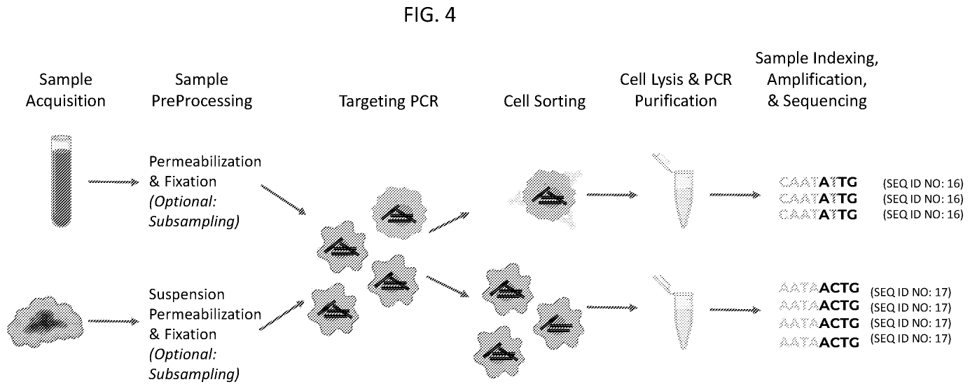

FIG. 5 provides a detailed workflow of an aspect of the present disclosure.

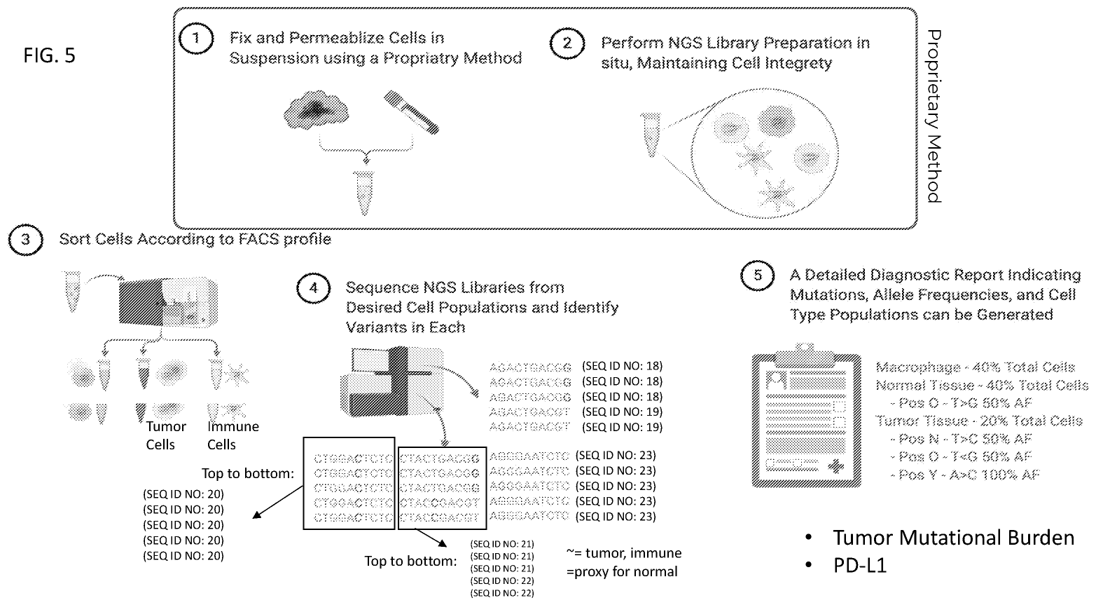

FIG. 6 provides a workflow diagram showing an amplification (e.g., rhAmpSeq as a non-limiting example, but any amplification method can be used) or ligation method (e.g., Hybrid Capture for DNA as a non-limiting example) for in Situ library preparation and potential applications.

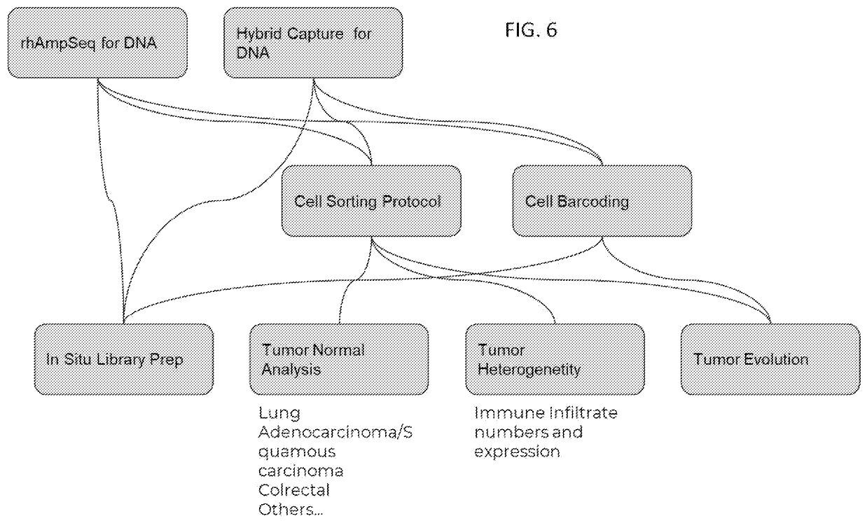

FIG. 7 provides an overview of potential aspects in the in Situ Library Preparation workflow. This workflow illustrates how cells remain intact throughout the process. Cell samples can come from cell culture, tissue, blood, biopsiesetc. and can be processed to create a cell suspension. The cell suspension is then fixed and permeabilized, before adding reagents in one or multiple steps to the cell suspension. After amplicons are generated, cell sorting can be implemented to isolate a subset of the reaction, before being lysed and purified.

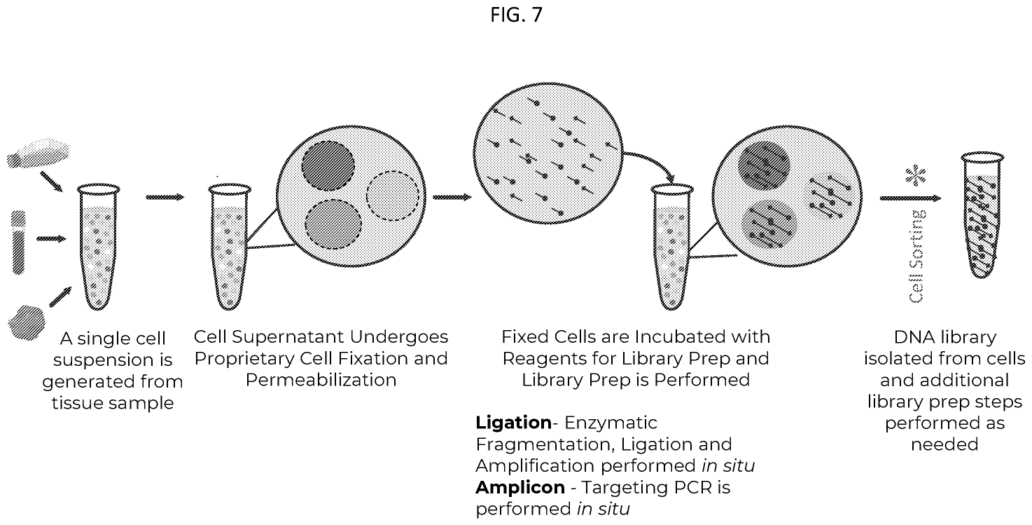

FIG. 8 shows amplified libraries prepared using the amplicon-based method of Example 1 that were run on a TapeStation HSD1000 (Agilent), showing product sized after the two PCR steps FIG. 8, panel (A). Libraries were not identical to gDNA, however appear to have amplification product in similar size ranges indicating amplification of the targets is occurring (fragments between 300 bp and 600 bp). The product around 180 bp was likely primer dimer. Sequencing libraries confirmed amplification of target amplicons FIG. 8, panel (B),

FIG. 9 shows amplicon-based library preparation (e.g., rhAmpSeq Library preparation (IDT)) of Example 2 performed on genomic DNA (gDNA) according to established manufacturer protocols. Amplified libraries were run on a TapeStation HSD1000 (Agilent), showing product sized after the two PCR steps.

FIG. 10 shows in situ amplicon library preparation performed on 16K and 32K fixed and permeabilized cells of Example 3. After PCR1, the cells were pelleted and resuspended in PBS, followed by sorting individual cells based on forward scatter and backscatter properties on a SONY SH800S, no dyes, stains or fluorophores were added to the cells. Subpopulations of 500, 1000, or 5000 cells were isolated, lysed and amplified using indexed primers. Amplicon products were ran on a TapeStation HSD1000 (Agilent), indicating amplification product in all subpopulations.

FIG. 11 shows in situ amplicon library preparation performed on two populations of fixed and permeabilized cells of Example 4. After PCR1, the cells were pelleted and resuspend in cell staining buffer (Biolegend) and then stained according to the experiment protocol below for either CD45-PE or IgG-PE. Cells were mixed and then sorted on a SONY SH800S based on PE fluorescence intensity. FIG. 11, panel (A) contained a histogram of the fluorescence intensities, FIG. 11, panel (B) contained cell numbers and percentages total observed, and FIG. 11, panel (C) showed size profile of the library after PCR2 amplification with TapeStation HSD1000 (Agilent).

FIG. 12 provides an example of In Situ ligation based Library preparation performed on genomic DNA (gDNA) according to established manufacturer protocols. An in situ protocol was developed and performed on in situ cells (in situ) (see experiment protocols of Example 5). After which the cells were lysed and amplified, libraries were purified and then run on a TapeStation HSD5000 (Agilent), showing product sized after the library preparation. FIG. 12, panel (A). Libraries are not identical to gDNA, due to differences in efficiency of enzymatic fragmentation, however amplification products are present in the samples, as indicated by the gel. Samples were then sequenced to confirm these products contain the required sequences for Illumina sequencing. And 99% of reads sequenced are mapping to the human genome FIG. 12, panel (B). Genome coverage is low, however, that is due to sequencing depth, which was low.

FIG. 13 shows in situ ligation library preparation of Example 6 performed on two populations of fixed and permeabilized cells. After the PCR step, the cells were pelleted and resuspend in cell staining buffer (Biolegend) and then stained according to the experiment protocol below for either CD45-PE or IgG-PE. Cells were mixed and then sorted on a SONY SH800S based on PE fluorescence intensity. FIG. 13, panel (A) contains a histogram of the fluorescence intensities, FIG. 13, panel (B) contains cell numbers and percentages total observed, and FIG. 13, panel (C) shows size profile of the library after PCR2 amplification with TapeStation HSD5000 (Agilent).

FIG. 14 provides a non-limiting example of the steps of the amplicon-based method of the present disclosure as compared to the ligation-based method of the present methods.

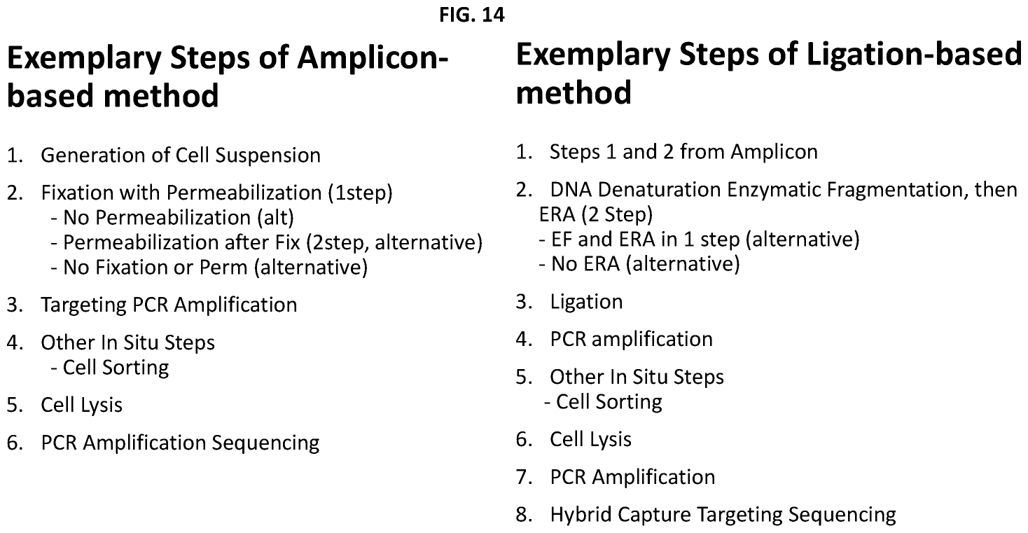

FIG. 15 provides a non-limiting example of amplicon-based method steps and alternatives or additional steps of the present disclosure.

FIG. 16 provides a non-limiting example of ligation-based method steps and alternatives or additional steps of the present disclosure.

### 5. DEFINITIONS

All publications, patents and patent applications cited herein, whether supra or infra, are hereby incorporated by reference in their entireties.

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a primer” includes a mixture of two or more such primers, and the like. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The terms “cytometry” and “flow cytometry” are also used consistent with their customary meanings in the art. In particular, the term “cytometry” can refer to a technique for identifying and/or sorting or otherwise analyzing cells. The term “flow cytometry” can refer to a cytometric technique in which cells present in a fluid flow can be identified, and/or sorted, or otherwise analyzed. Flow cytometry can be used in conjunction with standard methods to identify cells of interest, e.g., by labeling them with fluorescent markers and detecting the fluorescent markers via laser excitation. The terms “about” and “substantially” as used herein to denote a maximum variation of 10%, or 5%, with respect to a property including numerical values. Cytometry is, in some cases, used as a catch all to cover any known methods for identifying and sorting two or more populations of cells and can include magnetic activated cell sorting, and the like.

The practice of the present disclosure will employ, unless otherwise indicated, conventional methods of medicine, chemistry, biochemistry, immunology, cell biology, molecular biology and recombinant DNA techniques, within the skill of the art. Such techniques are explained fully in the literature. See, e.g., T Cell Protocols (Methods in Molecular Biology, G. De Libero ed., Humana Press; 2.sup.nd edition, 2009); C. W. Dieffenbach and G. S. Dveksler, PCR Primer: A Laboratory Manual (Cold Spring Harbor Laboratory Press; 2.sup.nd Lab edition, 2003); Next Generation Sequencing: Translation to Clinical Diagnostics (L. C. Wong ed., Springer, 2013); Deep Sequencing Data Analysis (Methods in Molecular Biology, N. Shomron ed., Humana Press, 2013); Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company, 1993); A. L. Lehninger, Biochemistry (Worth Publishers, Inc., current addition); Sambrook et al., Molecular Cloning: A Laboratory Manual (3.sup.rd Edition, 2001); Methods In Enzymology (S. Colowick and N. Kaplan eds., Academic Press, Inc.).

“Substantially purified” generally refers to isolation of a substance (compound, polynucleotide, oligonucleotide, protein, or polypeptide) such that the substance comprises the majority percent of the sample in which it resides. Typically, in a sample, a substantially purified component comprises 50%, 80%-85%, or 90-95% of the sample. Techniques for purifying polynucleotides, oliognucleotides, and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide or oligonucleotide is a nucleic acid molecule devoid, in whole or part, of sequences normally associated with it in nature; or a sequence, as it exists in nature, but having heterologous sequences in association therewith; or a molecule disassociated from the chromosome.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

A polynucleotide “derived from” a designated sequence refers to a polynucleotide sequence which comprises a contiguous sequence of approximately at least about 6 nucleotides, at least about 8 nucleotides, at least about 10-12 nucleotides, or at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription, or transcription, which is based on the information provided by the sequence of bases in the region(s) from which the polynucleotide is derived. As such, it may represent either a sense or an antisense orientation of the original polynucleotide.

“Recombinant” as used herein to describe a nucleic acid molecule means a polynucleotide of genomic, cDNA, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

As used herein, a “solid support” refers to a solid surface such as a magnetic bead, latex bead, microtiter plate well, glass plate, nylon, agarose, acrylamide, and the like.

As used herein, the term “target nucleic acid region” or “target nucleic acid” denotes a nucleic acid molecule with a “target sequence” to be amplified. The target nucleic acid may be either single-stranded or double-stranded and may include other sequences besides the target sequence, which may not be amplified. The term “target sequence” refers to the particular nucleotide sequence of the target nucleic acid which is to be amplified. The target sequence may include a probe-hybridizing region contained within the target molecule with which a probe will form a stable hybrid under desired conditions. The “target sequence” may also include the complexing sequences to which the oligonucleotide primers complex and extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands (or sense and antisense strands).

The term “primer” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is generally single-stranded for maximum efficiency in amplification but may alternatively be double-stranded. If double-stranded, the primer can first be treated to separate its strands before being used to prepare extension products. This denaturation step is typically affected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. A Primer can contain a sequence that hybridizes to the template strand only or also include additional sequences 5′ of the region that hybridizes to the template. These regions can include an indexing sequence, and/or an adapter sequence.

The term “adapter”, as used herein, refers to a fully or partially double stranded molecule that can be ligated to another molecule. An adapter can include a Y-adapter, hairpin adapter, full double stranded, and the like. The adapter is minimally composed of a common sequence that can be used for sequencing or further amplification of the library. The term “adapter sequence” is used to refer to the common sequence added on with adapters or PCR primers.

The term “binding” as used herein, refers to any form of attaching or coupling two or more components, entities, or objects. For example, two or more components may be bound to each other via chemical bonds, covalent bonds, ionic bonds, hydrogen bonds, electrostatic forces, Watson-Crick hybridization, etc.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. PCR reaction volumes typically range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g., Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” or “first set of primers” in reference to a nested amplification reaction mean the primers used to generate a first amplicon, and “secondary primers” or “second set of primers” mean the one or more primers used to generate a second, or nested, amplicon. In some embodiments, “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999) (two-color real-time PCR). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

The term “amplicon” or “amplified product” or “amplicon product” refers to the amplified nucleic acid product of a PCR reaction or other nucleic acid amplification process. The “amplicon product” refers to a segment of nucleic acid generated by an amplification process such as the PCR process or other nucleic acid amplification process such as ligation (e.g., ligase chain reaction). The terms are also used in reference to RNA segments produced by amplification methods that employ RNA polymerases, such as NASBA, TMA, etc. (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specific PCR, inverse PCR (see, e.g., Triglia, et al., (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis. It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

The “melting temperature” or “Tₘ” of double-stranded DNA is defined as the temperature at which half of the helical structure of DNA is lost due to heating or other dissociation of the hydrogen bonding between base pairs, for example, by acid or alkali treatment, or the like. The T.sub.m of a DNA molecule depends on its length and on its base composition. DNA molecules rich in GC base pairs have a higher T.sub.m than those having an abundance of AT base pairs. Separated complementary strands of DNA spontaneously reassociate or anneal to form duplex DNA when the temperature is lowered below the T.sub.m. The highest rate of nucleic acid hybridization occurs approximately 25 degrees C. below the T.sub.m. The T.sub.m may be estimated using the following relationship: T.sub.m=69.3+0.41(GC) (Marmur et al. (1962) J. Mol. Biol. 5:109-118).

The term “barcode” refers to a nucleic acid sequence that is used to identify a single cell or a subpopulation of cells. Barcode sequences can be linked to a target nucleic acid of interest during amplification or ligation and used to trace back the DNA or RNA to the cell or population from which the target nucleic acid originated. A barcode sequence can be added to a target nucleic acid of interest during amplification by carrying out PCR with a primer that contains a region comprising the barcode sequence and a region that is complementary to the target nucleic acid such that the barcode sequence is incorporated into the final amplified target nucleic acid product (i.e., amplicon). Barcodes can be included in either the forward primer or the reverse primer or both primers used in PCR to amplify a target nucleic acid. Alternatively, barcoding sequences can be included into barcoding adapters can be ligated onto a DNA or RNA target region using a ligation-based method. The term “barcode” or barcoding sequence” is used interchangeably herein as “indexing sequence”, “index” or “indexing”. In some embodiments, the barcode sequence refers to a sequence of 4-20 base pairs (bp) that is used to identify the origin of a sample, or population. The barcoding sequence on its own or in combination with another indexing sequence is a unique identifier (e.g., in a pool) of the specific sample or population being sequenced. In some embodiments, the indexing sequence is a sequence that is inserted in between two different consensus regions in adapters or primers.

The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; birds; and laboratory animals, including rodents such as mice, rats and guinea pigs, and the like. The term does not denote a particular age. Thus, both adult, newborn, and embryonic individuals are intended to be covered.

“Encode,” as used in reference to a nucleotide sequence of nucleic acid encoding a gene product, e.g., a protein, of interest, is meant to include instances in which a nucleic acid contains a nucleotide sequence that is the same as the endogenous sequence, or a portion thereof, of a nucleic acid found in a cell or genome that, when transcribed and/or translated into a polypeptide, produces the gene product.

“Target nucleic acid” or “target nucleotide sequence,” as used herein, refers to any nucleic acid or nucleotide sequence that is of interest for which the presence and/or expression level in a single cell or a cell within a cell population is sought using a method of the present disclosure. A target nucleic acid may include a nucleic acid having a defined nucleotide sequence (e.g., a nucleotide sequence encoding a cytokine), or may encompass one or more nucleotide sequences encoding a class of proteins.

“Originate,” as used in reference to a source of an amplified piece of nucleic acid, refers to the nucleic acid being derived either directly or indirectly from the source, e.g., a well in which a single T cell is sorted. Thus in some cases, the origin of a nucleic acid obtained as a result of a sequential amplification of an original nucleic acid may be determined by reading barcode sequences that were incorporated into the nucleic acid during an amplification step performed in a location that can in turn be physically traced back to the single T cell source based on the series of sample transfers that was performed between the sequential amplification steps.

The term “population”, e.g., “cell population” or “population of cells”, as used herein means a grouping (i.e., a population) of one or more cells that are separated (i.e., isolated) from other cells and/or cell groupings. For example, a 6-well culture dish can contain 6 cell populations, each population residing in an individual well. The cells of a cell population can be, but need not be, clonal derivatives of one another. A cell population can be derived from one individual cell. For example, if individual cells are each placed in a single well of a 6-well culture dish and each cell divides one time, then the dish will contain 6 cell populations. The cells of a cell population can be, but need not be, derived from more than one cell, i.e. non-clonal. The cells from which a non-clonal cell population may be derived may be related or unrelated and include but are not limited to, e.g., cells of a particular tissue, cells of a particular sample, cells of a particular lineage, cells having a particular morphological, physical, behavioral, or other characteristic, etc. A cell population can be any desired size and contain any number of cells greater than one cell. For example, a cell population can be 2 or more, 10 or more, 100 or more, 1,000 or more, 5,000 or more, 104 or more, 105 or more, 106 or more, 107 or more, 108 or more, 109 or more, 1010 or more, 1011 or more, 1012 or more, 1013 or more, 1014 or more, 1015 or more, 1016 or more, 1017 or more, 1018 or more, 1019 or more, or 1020 or more cells.

A “heterogeneous” cell population may include one or more cell populations, where each cell population contains cells that are phenotypically distinct from other cell populations.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the primer” includes reference to one or more primers and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### 6. DETAILED DESCRIPTION

Aspects of the present disclosure relate generally to methods, compositions, and kits for preparing a ligation- or amplicon-based library in situ for sequencing.

Further aspects of the present disclosure relate generally to methods, compositions, and kits for determining the heterogeneity of cell populations in a sample and identifying disease-associated genetic alterations of distinct cell populations within the sample. Aspects of the present disclosure also include a computer readable-medium and a processor to carry out the steps of the method or instructions of the kit described herein.

Further aspects of the present methods include preparation of the sample and/or fixation of the cells of the sample performed in such a manner that the prepared cells of the sample maintain characteristics of the unprepared cells, including characteristics of unprepared cells in situ, i.e., prior to collection, and/or unfixed cells following collection but prior to fixation and/or permeabilization and/or labeling. Keeping cells intact during library preparation using the methods described herein preserves the natural structure of the cells during library preparation.

### 6.1 Methods

### 6.1.1 Ligation-Based Library In Situ

Aspects of the present disclosure provide methods include preparing a ligation-based library preparation method in situ for sequencing, a ligation-based library in situ for sequencing.

Performing library preparation inside cells in situ allows for one to perform NGS library preparation inside of a multitude of individual cells within one reaction. This is a platform technology with a range of potential applications including cancer diagnostics, prenatal diagnostics, and profiling the microbiome, and it will aid sequencing of rare subpopulations by leveraging the ability to enrich the cell populations after library preparation.

The method for preparing a ligation-based library in situ for sequencing includes (a) providing a sample comprising a cell/nucleic population; (b) performing, in each cell/nuclei of the cell/nuclei population, an enzymatic fragmentation reaction to form DNA fragments within the cell/nuclei population; (c) ligating, in each cell/nuclei, the DNA fragments to adapter sequences to create a ligated library comprising ligated DNA fragments; (d) lysing each of the cells to collect the ligated DNA fragments; (e) purifying the ligated DNA fragments; and (f) sequencing the ligated DNA fragments.

### 6.1.1.1 Enzymatic Fragmentation Reaction

In some embodiments, the method includes contacting the cell/nuclei population with a fragmentation buffer and a fragmentation enzyme to form an enzymatic fragmentation mixture. Performing a enzymatic fragmentation reaction in the present ligation-based method provides for generating smaller sized DNA or RNA fragments containing the target region of interest. Methods for fragmenting DNA or RNA can include mechanical or enzyme-based fragmenting. Mechanical shearing methods include acoustic shearing, hydrodynamic shearing and nebulization, while enzyme-based methods include transposons, restriction enzymes and nicking enzymes. Any standard enzymatic fragmentation buffer and enzymatic fragmentation enzyme can be used for fragmenting the DNA or RNA.

In some embodiments, the one or more cell populations, the fragmentation buffer, and fragmentation enzyme are pipetted into a test tube. In some embodiments, the test tube is on ice.

In certain embodiments, the method optionally includes denaturing, by heat, prior to enzymatic fragmentation to improve fragmentation, likely by opening the chromatin structure of DNA or RNA in the cell/nuclei population. In alternative embodiments, the heat denaturation step is not performed prior to enzymatic fragmentation.

In some embodiments, the cell/nuclei population within the enzymatic fragmentation mixture is diluted to a volume of about 0.5 μl or more, about 1 μl or more, about 1.5 μl or more, about 2 μl or more, about 2.5 μl or more, about 3 μl or more, about 3.5 μl or more, about 4 μl or more, about 4.5 μl or more, about 5 μl or more, about 6 μl or more, about 7 μl or more, about 8 μl or more, about 9 μl or more, about 10 μl or more, about 11 μl or more, about 12 μl or more, about 13 μl or more, about 14 μl or more, about 15 μl or more, about 16 μl or more, about 17 μl or more, about 18 μl or more, about 19 μl or more, about 20 μl or more, about 25 μl or more, about 30 μl or more, about 35 μl or more, about 40 μl or more, about 45 μl or more, about 50 μl or more, about 55 μl or more, or about 60 μl or more.

In some embodiments, the cell/nuclei population in the enzymatic fragmentation mixture is diluted to contain 1 to 1,000,000 cells. In some embodiments, the cell/nuclei population is diluted to contain 1 to 20,000 cells. In some embodiments, the cell/nuclei population is diluted to contain 1 to 16,000 cells. In some embodiments, the cell/nuclei population is diluted to contain 1 to 15,000 cells. In some embodiments, the cell/nuclei population is diluted to contain 1 to 10,000 cells. In some embodiments, the cell/nuclei population is diluted to contain 1 to 100 cells, 100 to 200 cells, 200 to 300 cells, 300 to 400 cells 400 to 500 cells, 500 to 600 cells, 600 to 700 cells, 700 to 800 cells, 800 to 900 cells, 900 to 1000 cells, 1000 to 1100 cells, 1100 to 1200 cells, 1200 to 1300 cells, 1300 to 1400 cells, or 1400 to 1500 cells. In some embodiments, the cell/nuclei population is diluted to contain 20,000 cells or less, 19,000 cells or less, 18,000 cells or less, 17,000 cells or less, 16,000 cells or less, 15,000 cells or less, 14,000 cells or less, 13,000 cells or less, 12,000 cells or less, 11,000 cells or less, 10,000 cells or less, 9,000 cells or less, 8,000 cells or less, 7,000 cells or less, 6,000 cells or less, 5,000 cells or less, 4,000 cells or less, 3,000 cells or less, 2,000 cells or less, 1,500 cells or less, 1,000 cells or less, 500 cells, 250 cells or less, 100 cells or less, 50 cells or less, 25 cells or less, 10 cells or less, 5 cells or less, or 2 cells or less. In some embodiments, the cell/nuclei population is diluted to contain 1 cell. In some embodiments, the cell/nuclei population is diluted to contain 1 to 15,000 cells. In some embodiments, the cell/nuclei population is diluted to contain 1 to 300 cells, 1 to 10 cells, 3 to 10 cells, 10 to 20 cells, 1 to 5 cells, 1 to 15 cells, 1 to 25 cells, 1 to 75 cells, and the like.

In certain embodiments, the enzymatic fragmentation mixture does not include EDTA. In certain embodiments, the enzymatic fragmentation mixture includes EDTA.

In some embodiments, the fragmentation enzyme is selected from a KAPA fragmentation enzyme, TaKara fragmentation enzyme, NEBNext Ultra enzymatic fragmentation enzyme, biodynamic DNA Fragmentation Enzyme Mix, KAPA Fragmentation Kit for Enzymatic Fragmentation, and the like. In some embodiments, the fragmentation enzyme is a Caspase-Activated DNase (CAD). In some embodiments, a fragmentation enzyme and fragmentation buffer are contacted with cell/nuclei population in an amount sufficient to perform a fragmentation reaction. In some embodiments, the volume of fragmentation enzyme added to the sample containing cell/nuclei population ranges from 10 μl to 100 μl. In some embodiments, the volume of fragmentation enzyme added to the sample containing cell/nuclei population ranges from 1 μl to 20 μl, 1 μl to 5 μl, 5 μl to 10 μl, 5 μl to 15 μl, or 8 μl to 12 μl. In certain embodiments, the volume of fragmentation enzyme added to the sample containing cell/nuclei population is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, or 20 μl or more.

In some embodiments, the fragmentation buffer is selected from aKAPA fragmentation buffer, TaKara fragmentation buffer, NEBNext Ultra enzymatic fragmentation buffer, biodynamic DNA Fragmentation buffer, KAPA Fragmentation buffer, and the like. However, any commercially available enzymatic fragmentation buffer can be used for fragmenting the DNA or RNA of the cell/nuclei.

In some embodiments, the final enzymatic fragmentation mixture comprises a volume ranging from 10 μl to 100 μl. In some embodiments, the fragmentation buffer is a KAPA fragmentation buffer. In some embodiments, the volume of fragmentation buffer added to the sample containing cell/nuclei population ranges from 10 μl to 100 μl. In some embodiments, the volume of fragmentation buffer added to the sample containing cell/nuclei population ranges from 1 μl to 20 μl, 1 μl to 5 μl, 5 μl to 10 μl, 5 μl to 15 μl, or 8 μl to 12 μl. In certain embodiments, the volume of fragmentation buffer added to the sample containing cell/nuclei population is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, or 70 μl or more.

In some embodiments, the final volume of the enzymatic fragmentation mixture containing one or more cells, a fragmentation buffer, and a fragmentation enzyme ranges from 5 μl to 100 μl. In some embodiments, the final volume of the enzymatic fragmentation mixture containing one or more cells, a fragmentation buffer, and a fragmentation enzyme is 10 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the enzymatic fragmentation mixture comprises a conditioning solution. In some embodiments, the volume of conditioning solution added to the enzymatic fragmentation mixture ranges from 1 μl to 20 μl. In some embodiments, the volume of 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, or 20 μl or more. In some embodiments, the conditioning solution is a solution that adjusts the enzymatic fragmentation buffer to handle highly sensitive reagent compositions, and in some cases sequesters EDTA (or other chelators) in the sample. In some embodiments, the conditioning solution contains a reagent that binds EDTA in the sample. In some embodiments, the conditioning solution contains Magnesium or other cations to bind to EDTA in the cell population. In some embodiments, the conditioning solution is a solution that binds to magnesium in the sample. In some embodiments, the conditioning solution contains a divalent cation chelator to bind to excess magnesium in the sample.

In some embodiments, the method includes performing enzymatic fragmentation on the nucleic acids (e.g., DNA or RNA) within the cell/nuclei population to form an enzymatic fragmentation reaction mixture. In some embodiments, performing an enzymatic fragmentation reaction on the mixture comprises loading the enzymatic fragmentation mixture onto a thermocycler. In some embodiments, performing an enzymatic fragmentation reaction on the mixture comprises loading the enzymatic fragmentation mixture onto a heat block.

In some embodiments, the method includes incubating the enzymatic fragmentation mixture in the thermocycler for a duration/time period ranging from 1 minute to 120 minutes, 3 minutes to 10 minutes, 5 minutes to 20 minutes, 10 minutes to 25 minutes, or 20 minutes to 40 minutes. In certain embodiments, the duration is 1 minute or more, 2 minutes or more, 3 minutes or more, 4 minutes or more, 5 minutes or more, 6 minutes or more, 7 minutes or more, 8 minutes or more, 9 minutes or more, 10 minutes or more, 15 minutes or more, 20 minutes or more, 25 minutes or more, 30 minutes or more, 35 minutes or more, 40 minutes or more, 45 minutes or more, 50 minutes or more, 55 minutes or more, or 60 minutes or more. In some embodiments, before fragmenting, the method includes a pre-incubation step to allowing the enzymes to enter the cell.

In some embodiments, performing an enzymatic fragmentation reaction on the mixture comprises loading the mixture onto a thermocycler and incubating the mixture at a temperature ranging from 2° C. to 80° C., such as 4° C. to 37° C., 4° C. to 50° C., or 5° C. to 40° C. In some embodiments, the method includes incubating the mixture in the thermocycler at a temperature of 2° C. or more, 3° C. or more, 4° C. or more, 5° C. or more, 6° C. or more, 7° C. or more, 8° C. or more, 9° C. or more, 10° C. or more, 15° C. or more, 20° C. or more, 25° C. or more, 30° C. or more, 35° C. or more, 40° C. or more, 45° C. or more, 50° C. or more, 55° C. or more, 60° C. or more, 65° C. or more, 70° C. or more, 75° C. or more, or 80° C. or more.

In some embodiments, before the ligating step (c) of the ligation-based method, the method includes performing an end-repair and/or A-tailing reaction on the one or more DNA or RNA fragments. In some embodiments the enzymatic fragmentation enzyme is heat inactivated before end repair and A (ERA) tailing (described below) at a known temperature for inactivating the specific enzyme 65-99.5*C for 5-60 minutes. In some embodiments the End repair and A tailing incubation step also acts as the heat inactivation step for enzymatic fragmentation enzymes.

In some embodiments, the End-repair and A-tailing reaction and the enzymatic fragmentation reaction occurs in a single reaction, with multiple temperature incubations. For example, the End repair and/or A-tailing reaction can occur during the enzymatic fragmentation reaction in a single reaction. In other embodiments, the End repair and/or A-tailing reaction can occur in different, separate reactions. In some embodiments, the End-repair and A-tailing reaction and the enzymatic fragmentation reaction are separate reactions.

### 6.1.1.2 End Repair and A-Tailing

In some embodiments, the method includes performing an End-repair and/or A-tailing reaction on the one or more fragmented DNA or RNA within the cell/nuclei population. End Repair and/or A-Tailing are two enzymatic steps configured to blunt the DNA or RNA fragments and, optionally, add an overhanging A nucleotide to the end of the DNA or RNA fragments, for example, to improve ligation efficiency. The end-repair and/or A-tailing reaction is performed before ligating the DNA or RNA fragments.

In some embodiments, the End Repair and/or A-tailing can occur in the same reaction as the enzymatic fragmentation reaction described above.

In some embodiments, performing an end-repair and/or A-tailing reaction comprises contacting the fragmented DNA or RNA within the cell/nuclei population with an End Repair A-tail buffer and an End Repair A-tail enzyme to form an End Repair A-tail mixture. In some embodiments, performing an End-repair and A-tailing reaction comprises contacting the fragmented DNA or RNA within the cell/nuclei population in the enzymatic fragmentation reaction mixture with an End Repair A-tail buffer and an End Repair A-tail enzyme to form an End Repair A-tail mixture. In some embodiments, contacting the fragmented DNA or RNA within the cell/nuclei population in the enzymatic fragmentation reaction mixture with an End Repair A-tail buffer and an End Repair A-tail enzyme occurs on ice.

In some embodiments, the fragmented DNA (e.g., double stranded DNA or single stranded DNA) or RNA within the End Repair A-tail mixture is diluted to a volume of about 0.5 μl or more, about 1 μl or more, about 1.5 μl or more, about 2 μl or more, about 2.5 μl or more, about 3 μl or more, about 3.5 μl or more, about 4 μl or more, about 4.5 μl or more, about 5 μl or more, about 6 μl or more, about 7 μl or more, about 8 μl or more, about 9 μl or more, about 10 μl or more, about 11 μl or more, about 12 μl or more, about 13 μl or more, about 14 μl or more, about 15 μl or more, about 16 μl or more, about 17 μl or more, about 18 μl or more, about 19 μl or more, about 20 μl or more, about 25 μl or more, about 30 μl or more, about 35 μl or more, about 40 μl or more, about 45 μl or more, about 50 μl or more, about 55 μl or more, about 60 μl or more, about 65 μl or more, about 70 μl or more, about 75 μl or more, about 80 μl or more, about 85 μl or more, about 90 μl or more, about 95 μl or more, or about 100 μl or more.

In some embodiments, the volume of End Repair A-tail enzyme added to the enzymatic fragmentation reaction mixture (e.g., containing the fragmented DNA or RNA) ranges from 1 μl to 20 μl, 1 μl to 5 μl, 5 μl to 10 μl, 5 μl to 15 μl, or 8 μl to 12 μl. In certain embodiments, the volume of fragmentation enzyme added to the sample containing cell/nuclei population is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, or 20 μl or more.

In some embodiments, the volume of End Repair A-tail buffer added to the enzymatic fragmentation reaction mixture (e.g., containing the fragmented DNA or RNA) ranges from 10 μl to 100 μl. In some embodiments, the volume of fragmentation buffer added to the sample containing cell/nuclei population ranges from 1 μl to 20 μl, 1 μl to 5 μl, 5 μl to 10 μl, 5 μl to 15 μl, or 8 μl to 12 μl. In certain embodiments, the volume of End Repair A-tail buffer added to the sample containing cell/nuclei population is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, or 70 μl or more.

In some embodiments, the final volume of the End Repair A-tail mixture containing one or more cells, an End Repair A-tail buffer, and an End Repair A-tail enzyme ranges from 5 μl to 100 μl. In some embodiments, the final volume of the End Repair A-tail mixture containing one or more cells, an End Repair A-tail buffer, and an End Repair A-tail enzyme is 10 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the method further comprises running the End Repair A-tail mixture in a thermocycler to form an End Repair A-tail reaction mixture.

In some embodiments, the End Repair A-tail mixture is incubated in the thermocycler at a temperature ranging from 2° C. to 90° C. In some embodiments, performing an End Repair A-tail reaction on the End Repair A-tail mixture comprises loading the End Repair A-tail mixture onto a thermocycler and incubating the End Repair A-tail mixture at a temperature ranging from 2° C. to 50° C., such as 4° C. to 37° C., 4° C. to 50° C., or 5° C. to 40° C. In some embodiments, the method includes incubating the End Repair A-tail mixture in the thermocycler at a temperature of 2° C. or more, 3° C. or more, 4° C. or more, 5° C. or more, 6° C. or more, 7° C. or more, 8° C. or more, 9° C. or more, 10° C. or more, 15° C. or more, 20° C. or more, 25° C. or more, 30° C. or more, 35° C. or more, 40° C. or more, 45° C. or more, 50° C. or more, 55° C. or more, 60° C. or more, 65° C. or more, 70° C. or more, 75° C. or more, 85° C. or more, 85° C. or more, 90° C. or more, 95° C. or more, or 100° C. or more.

In some embodiments, the End Repair A-tail mixture is incubated for a duration ranging from 5 minutes to 50 minutes. In some embodiments, the method includes incubating the End Repair A-tail mixture in the thermocycler for a duration/time period ranging from 1 minute to 50 minutes, 3 minutes to 10 minutes, 5 minutes to 20 minutes, 10 minutes to 25 minutes, or 20 minutes to 40 minutes. In certain embodiments, the duration is 1 minute or more, 2 minutes or more, 3 minutes or more, 4 minutes or more, 5 minutes or more, 6 minutes or more, 7 minutes or more, 8 minutes or more, 9 minutes or more, 10 minutes or more, 15 minutes or more, 20 minutes or more, 25 minutes or more, 30 minutes or more, 35 minutes or more, 40 minutes or more, 45 minutes or more, 50 minutes or more, 55 minutes or more, or 60 minutes or more. In some embodiments the End repair and A tail enzymes are heat inactivated before proceeding to ligation at 65-100° C. for 5-60 minutes or more.

### 6.1.1.3 Adapter-Indexing Ligation

The present ligation-based method includes ligating, in each cell, the DNA or RNA fragments to adapter sequences in situ to create a ligated library comprising ligated DNA or RNA fragments.

In some embodiments, ligating includes performing ligase chain reaction (LCR). The ligase chain reaction (LCR) is an amplification process that involves a thermostable ligase to join two probes or other molecules together. In some embodiments, the thermostable ligase can include, but is not limited to Pfu ligase, or a Taq ligase. In some embodiments, the ligated product is then amplified to produce an amplicon product. In some embodiments, LCR can be used as an alternative approach to PCR. In other embodiments, PCR can be performed after LCR.

Ligating the DNA fragments to the adapter sequences comprises running the DNA fragments and adapter sequences in a thermocycler at a temperature and duration sufficient to ligate the DNA fragmented to the adapter sequences. Ligation reagents and/or enzymes can be used for ligating the DNA or RNA fragments. In some embodiments, ligation chain reaction (LCR) can be used for ligating the DNA or RNA fragments.

The fragmented DNA or RNA are contacted with adapter sequences to form a ligated library/ligation mixture containing the ligated DNA or RNA fragments. In some embodiments, the ligation mixture can include a Ligation Master Mix. In some embodiments, the ligation mixture can include a Blunt/TA Ligase Master Mix.

Adapter Ligation enzymatically combines (e.g., ligates) adapters provided in the reaction to the prepared DNA or RNA fragments. Non-limiting examples of adapter sequences include, but are not limited to, adapter nucleotide sequences that allow high-throughput sequencing of amplified or ligated nucleic acids. In some embodiments, the adapter sequences are selected from one or more of: a Y-adapter nucleotide sequence, a hairpin nucleotide sequence, a duplex nucleotide sequence, and the like. In some embodiments, the adapter sequences are for pair-end sequencing. In some embodiments, the adapter sequences include sequencing read primer sequences (e.g., R1, R2, i5, i7 etc.). In some embodiments, the adapter sequences include sample barcodes. Adapter sequences can be used in a ligation reaction of the disclosed method for the desired sequencing method used.

In some embodiments, the ligation mixture includes the End-repair A-tail reaction mixture or enzymatic fragmentation reaction mixture, a set of adapter sequences, and a ligation master mix. In certain embodiments, ligation mixture includes the End-repair A-tail reaction mixture or enzymatic fragmentation reaction mixture, a set of adapter sequences, nuclease free H2O, and a ligation master mix. In certain embodiments, the ligation mixture includes a final volume ranging from 10 μl to 200 μl, such as 10 μl to 100 μl, 10 μl to 150 μl, 50 μl to 150 μl, 50 μl to 120 μl, 70 μl to 115 μl, or 90 μl to 110 μl. In certain embodiments, the ligation mixture includes a final volume of 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, 100 μl or more, 105 μl or more, 110 μl or more, 115 μl or more, 120 μl or more, 125 μl or more, 130 μl or more, 135 μl or more, 140 μl or more, 145 μl or more, 150 μl or more, 155 μl or more, 160 μl or more, 165 μl or more, 170 μl or more, 175 μl or more, 180 μl or more, 185 μl or more, 190 μl or more, 195 μl or more, or 200 μl or more.

In some embodiments, the ligation mixture includes the enzymatic fragmentation mixture (e.g., when End-repair A tail is included in the enzymatic fragmentation reaction) in a volume ranging from 1 μl to 100 μl. In some embodiments, the ligation mixture includes the enzymatic fragmentation mixture in a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the ligation mixture includes the End-repair A-tail reaction mixture or enzymatic fragmentation mixture in a volume ranging from 1 μl to 100 μl. In some embodiments, the ligation mixture includes the End-repair A-tail reaction mixture or enzymatic fragmentation mixture in a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the ligation mixture includes the set of adapter sequences in a volume ranging from 1 μl to 20 μl, 1 μl to 5 μl, or 1 μl to 10 μl. In some embodiments, the ligation mixture includes the set of adapter sequences in a volume of 1 μl or more, 1.5 μl or more, 2 μl or more, 2.5 μl or more, 3 μl or more, 3.5 μl or more, 4 μl or more, 4.5 μl or more, 5 μl or more, 5.5 μl or more, 6 μl or more, 6.5 μl or more, 7 μl or more, 7.5 μl or more, 8 μl or more, 8.5 μl or more, 9 μl or more, 9.5 μl or more, 10 μl or more, 11 μl or more, 12 μl or more, 13 μl or more, 14 μl or more, 15 μl or more, or 20 μl or more.

In some embodiments, the nuclease free H₂O in the ligation mixture comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, or 15 μl or more. In some embodiments the nuclease free H₂O is replaced with a buffered solution (e.g., such as PBS).

In some embodiments, the ligation master mix comprises nuclease free H2O, a ligation buffer, and a DNA ligase. In some embodiments, the ligation master mix includes a final volume ranging from 5 μl to 100 μl, such as 10 μl to 50 μl, 25 μl to 50 μl, or 30 μl to 60. In some embodiments, the ligation master mix includes a final volume of 10 μl or more, 11 μl or more, 12 μl or more, 13 μl or more, 14 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the nuclease free H₂O in the ligation master mix comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, or 15 μl or more.

In some embodiments, the ligation buffer in the ligation master mix comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, or 70 μl or more.

In some embodiments, the DNA ligase in the ligation master mix comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 20 μl or more, 25 μl or more, 30 μl or more, 35 μl or more, 40 μl or more, 45 μl or more, 50 μl or more, 55 μl or more, 60 μl or more, 65 μl or more, or 70 μl or more.

In certain embodiments, the method comprises preparing the ligation master mix to a final volume ranging from 10 μl to 100 μl. In some embodiments, the final volume of the ligation master mix ranges from 1 μl to 20 μl, 1 μl to 5 μl, 5 μl to 10 μl, 5 μl to 15 μl, or 8 μl to 12 μl. In certain embodiments, the final volume of the ligation master mix is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the method includes ligating the fragmented DNA or RNA to the adapter sequences. In certain embodiments, ligating the fragmented DNA or RNA to the adapter sequences comprises running the ligation mixture in the thermocycler at a temperature and duration sufficient to ligate the fragmented DNA or RNA to the adapter sequences, such as, but not limited to: barcoding sequences, consensus read regions for sequencing, adapter sequences, or other indexing sequences for the sequencing method being used.

In some embodiments, the temperature ranges from 4° C. to 90° C. In some embodiments, the method includes incubating the ligation mixture in the thermocycler at a temperature of 2° C. or more, 3° C. or more, 4° C. or more, 5° C. or more, 6° C. or more, 7° C. or more, 8° C. or more, 9° C. or more, 10° C. or more, 15° C. or more, 20° C. or more, 25° C. or more, 30° C. or more, 35° C. or more, 40° C. or more, 45° C. or more, 50° C. or more, 55° C. or more, 60° C. or more, 65° C. or more, 70° C. or more, 75° C. or more, 85° C. or more, 85° C. or more, 90° C. or more, 95° C. or more, or 100° C. or more.

In some embodiments, the duration ranges from 5 minutes to 4 hours. In some embodiments, the method includes incubating the ligation mixture in the thermocycler for a duration/time period ranging from 1 minute to 5 hours, 1 minute to 4 hours, 1 minute to 50 minutes, 3 minutes to 10 minutes, 5 minutes to 20 minutes, 10 minutes to 25 minutes, or 20 minutes to 40 minutes. In certain embodiments, the duration is 1 minute or more, 2 minutes or more, 3 minutes or more, 4 minutes or more, 5 minutes or more, 6 minutes or more, 7 minutes or more, 8 minutes or more, 9 minutes or more, 10 minutes or more, 15 minutes or more, 20 minutes or more, 25 minutes or more, 30 minutes or more, 35 minutes or more, 40 minutes or more, 45 minutes or more, 50 minutes or more, 55 minutes or more, or 60 minutes or more. In certain embodiments, the duration is 1 hour or more, 1.5 hours or more, 2 hours or more, 2.5 hours or more, 3 hours or more, 3.5 hours or more, 4 hours or more. 4.5 hours or more, or 5 hours or more.

In some embodiments the ligase enzyme is heat inactivated at a temperature ranging from 65-99.5° C. for a duration ranging from 5-60 minutes before proceeding to the next steps. In some embodiments, ligase enzymes do not need to be heat inactivated.

### 6.1.1.4 Examples of Additional Amplification of Ligated Library

In some embodiments of the in situ ligation-based library preparation method, after the ligation step, but before lysing the cells to collect the ligated DNA or RNA fragments, the method further comprises amplifying the ligated DNA or RNA fragments to form amplicon products. Amplifying the ligated DNA or RNA fragments allows for to creating more copies of the DNA or RNA fragments, reducing the likelihood of region drop out due to in efficiencies in purification and/or hybridization capture protocols. Additionally, the method allows for adding additional sequences such as adapter sequences, read sequences, full primer sequences with sample barcodes, and the like during amplification. In some embodiments, amplifying the ligated DNA or RNA fragments to form amplicon products comprises contacting the ligated DNA or RNA fragments with amplification primers (e.g., primers used to hybridize with sample DNA or RNA that define the region to be amplified, but can also include, barcoding primers, P5/P7 primers, R1/R2 primers, other sequencing primers, and the like).

Additionally, multiple PCR reactions may be m performed, for example, after ligation but before sequencing the ligated DNA or RNA fragments of the cells. Some, none, or all of these additional PCR steps could occur before cell lysis, while some, none, or all of these additional PCR steps could occur after cell lysis. Additional PCR steps can include adding additional components to a PCR reaction, with each addition defined as a “PCR step”. For example, adding targeting primers, followed by adding amplification primers can take place in two PCR reactions, e.g. two PCR steps or one PCR reaction, e.g., one PCR step. In some embodiments, one or more, two or more, three or more, four or more, five or more, six or more, seven or more, eight or more, nine or more, or ten or more distinct PCR reactions can be performed. In certain embodiments, two PCR reactions are performed between ligation and sequencing steps (e.g., after ligation, but before lysing). In certain embodiments, three PCR reactions are performed between ligation and sequencing steps (e.g., after ligation, but before lysing). In certain embodiments, four PCR reactions are performed between ligation and sequencing steps (e.g., after ligation, but before lysing). In certain embodiments, the PCR reactions are performed after ligation but before the lysing step. In certain embodiments, the PCR reactions are performed after ligation but before the lysing step.

When performing amplification after the ligation step, the method includes contacting the ligated library (e.g., adapter ligated DNA or RNA fragments) with primers. In some embodiments, the method includes amplifying the ligated library with primers containing minimal sequences (e.g., read 1, read 2 sequences, P5 and/or P7 sequences, etc.). In some embodiments, the method includes amplifying the ligated library with primers including sample barcodes. In some embodiments, the method includes amplifying the ligated library with primers including the sequencing adapters, such as P5 and P7.

In some embodiments, the method includes amplifying the adapter-ligated fragments (e.g., ligated library) to create more copies before going through hybridization capture and/or sequencing. In some embodiments, the method includes amplifying the adapter-ligated fragments to add full length adapter sequences onto the adapter-ligated fragments, if necessary.

In some embodiments, after the ligating step to produce the ligated library but before sequencing, the method includes contacting the ligated library with an amplification mixture. In some embodiments, the amplification mixture comprises any readily available, standard amplification library mix or one or more components thereof, a set of amplification primers, and the adapter-ligated library. In some embodiments, the amplification mixture comprises a KAPA HiFi Hotstart Ready Mix (2×) or one or more components from the ready mix thereof, a set of amplification primers, and the adapter-ligated library. In some embodiments, the amplification mixture comprises a xGen Library Amplification Primer Mix or one or more components from the primer mix thereof, a set of amplification primers, and the adapter-ligated library. In other embodiments, the amplification mixture includes a Library Amplification Hot Start Master Mix and a xGen UDI primer Mix (IDT).

In some embodiments, the amplification mixture comprises a total volume ranging from 10 to 100 μl. In some embodiments, the final volume of the amplification mixture ranges from 1 μl to 50 μl, 1 μl to 30 μl, 1 μl to 25 μl, 1 μl to 5 μl, 5 μl to 10 μl, 5 μl to 15 μl, or 8 μl to 12 μl. In certain embodiments, the final volume of the amplification mixture is 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the amplification library mix (e.g., KAPA HiFi Hotstart Ready Mix (2×), xGen Library Amplification Primer Mix, or Amplification Hot Start Master Mix) within the amplification mixture comprises a volume ranging from 1 to 100 μl. In some embodiments, the amplification library mix within the amplification mixture ranges from 1 μl to 20 μl, 1 μl to 5 μl, 5 μl to 10 μl, 5 μl to 15 μl, or 8 μl to 12 μl. In certain embodiments, the amplification library mix within the amplification mixture comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the set of amplification primers within the amplification mixture comprises a volume ranging from 10 to 100 μl. In some embodiments, the set of amplification primers within the amplification mixture ranges from 1 μl to 20 μl, 1 μl to 5 μl, 5 μl to 10 μl, 5 μl to 15 or 8 μl to 12 In certain embodiments, the set of amplification primers within the amplification mixture comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the Library Amplification Hot Start Master Mix within the amplification mixture comprises a volume ranging from 1-100 μl. In some embodiments, the Library Amplification Hot Start Master Mix within the amplification mixture comprises a volume of about 10 μl, 15 μl, 20 μl, 25 μl, 30 μl, 35 μl, 40 μl, 45 μl, 50 μl, 55 μl, 60 μl, 65 μl, 70 μl, 75 μl, 80 μl, 85 μl, 90 μl, 95 μl, or 100 μl.

In some embodiments, the primer Mix within the amplification mixture comprises a volume ranging from 1-10 μl. In some embodiments, the primer Mix (IDT) within the amplification mixture comprises a volume of about 1 μl, 2 μl, 3 μl, 4 μl, 5 μl, 6 μl, 7 μl, 8 μl, 9 μl, or about 10 μl.

In some embodiments, the ligated library within the amplification mixture comprises a volume ranging from 10 to 100 μl. In some embodiments, the ligated library within the amplification mixture ranges from 1 μl to 20 μl, 1 μl to 5 μl, 5 μl to 10 μl, 5 μl to 15 μl, or 8 μl to 12 μl. In certain embodiments, the ligated library within the amplification mixture comprises a volume of 1 μl or more, 2 μl or more, 3 μl or more, 4 μl or more, 5 μl or more, 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, 20 μl or more, 25 μl or more, 30 μl or more, 35 μl or more, 40 μl or more, 45 μl or more, 50 μl or more, 55 μl or more, 60 μl or more, 65 μl or more, 70 μl or more, 75 μl or more, 80 μl or more, 85 μl or more, 90 μl or more, 95 μl or more, or 100 μl or more.

In some embodiments, the method comprises amplifying the amplification mixture to produce a first set of amplicon products. In some embodiments, amplifying is performed using a thermocycler. In some embodiments, amplifying is performed using polymerase chain reaction (PCR).

In some embodiments, amplifying comprises running the amplification mixture in the thermocycler for a duration ranging from 1 second to 5 minutes. In some embodiments, amplifying comprises running the amplification mixture in the thermocycler for a duration ranging from 1 second to 1 minute. In some embodiments, amplifying comprises running the amplification mixture in the thermocycler for a duration ranging from 30 seconds to 1 minute. In some embodiments, amplifying comprises running the amplification mixture in the thermocycler for a duration ranging from 45 seconds to 1 minute. In some embodiments, amplifying comprises running the amplification mixture in the thermocycler for a duration of 1 second or more, 5 seconds or more, 15 seconds or more, 20 seconds or more, 25 seconds or more, 30 seconds or more, 35 seconds or more, 40 seconds or more, 45 seconds or more, 50 seconds or more, 55 seconds or more, 60 seconds or more, 1 minute or more, or 1.5 minutes or more.

In some embodiments, the temperature of incubation of the amplification mixture in the thermocycler ranges from 4° C. to 110° C. In some embodiments, the method includes incubating the amplification mixture in the thermocycler at a temperature of 2° C. or more, 3° C. or more, 4° C. or more, 5° C. or more, 6° C. or more, 7° C. or more, 8° C. or more, 9° C. or more, 10° C. or more, 15° C. or more, 20° C. or more, 25° C. or more, 30° C. or more, 35° C. or more, 40° C. or more, 45° C. or more, 50° C. or more, 55° C. or more, 60° C. or more, 65° C. or more, 70° C. or more, 72° C. or more, 75° C. or more, 85° C. or more, 85° C. or more, 90° C. or more, 95° C. or more, 100° C. or more, 105° C. or more, 110° C. or more, 115° C. or more, 120° C. or more, 125° C. or more, 130° C. or more, 140° C. or more, 145° C. or more, or 150° C. or more.

### 6.1.1.5 Lysing the Cells to Collected Ligated DNA or RNA Fragments

Aspects of the present ligation-based method include lysing each of the cells to collect the ligated and/or amplified DNA or RNA fragments.

The lysing step can be accomplished by contacting the DNA or RNA fragments within the cell with a cell lysing agent or physically disrupting the cell structure. In some embodiments, said lysing occurs after the ligation step. In some embodiments, lysing occurs after one or more PCR steps. In some embodiments, lysing occurs after a sorting step. Lysing the cells with a cell lysing agent facilitates purification and isolation of the DNA or RNA fragments for each cell/nuclei population.

Lysing the cells breaks open the cells, and in some cases. also breaks down the proteins in the cells leaving the ligated DNA or RNA behind (e.g., ligated DNA or RNA fragments).

Non-limiting examples of cell lysing agents include, but are not limited to, an enzyme solution, physical manipulation, or chemical methods. In some embodiments, the lysis solution includes a proteases or proteinase K, phenol and guanidine isothiocyanate, RNase inhibitors, SDS, sodium hydroxide, potassium acetate, and the like. However, any known cell lysis buffer may be used to lyse the cells within the one or more cell populations. Physical methods include mechanical shearing or repeated freeze thaws. Chemical denaturation includes use of detergents, chaotropic, or hypotonic solutions.

In some embodiments, lysing includes heating the cells for a period of time sufficient to lyse the cells. In certain embodiments, the cells can be heated to a temperature of about 25° C. or more, 30° C. or more, 35° C. or more, 37° C. or more, 40° C. or more, 45° C. or more, 50° C. or more, 55° C. or more, 60° C. or more, 65° C. or more, 70° C. or more, 80° C. or more, 85° C. or more, 90° C. or more, 96° C. or more, 97° C. or more, 98° C. or more, or 99° C. In certain embodiments, the cells can be heated to a temperature of about 90° C., 95° C., 96° C., 97° C., 98° C., or 99° C.

### 6.1.1.6 Additional Exemplary Steps

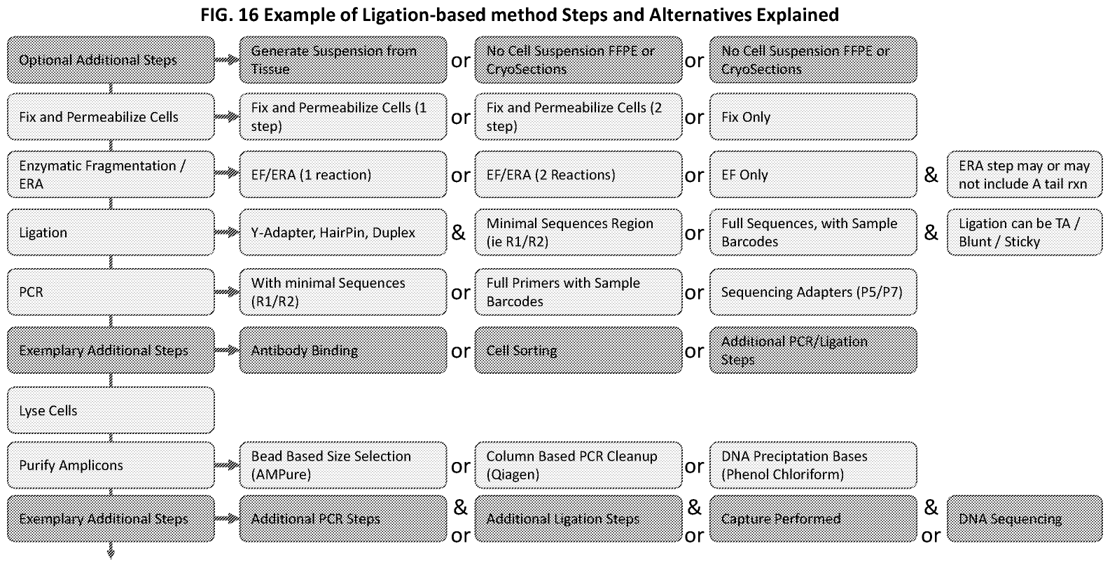

Aspects of the present ligation-based methods include additional exemplary steps, for example, as shown in FIG. 16.

The present ligation-based methods include some additional steps that can be performed either before or after performing the lysing step, according to the step. Some or all of these steps do not occur in some embodiments.

### Barcoding

In some embodiments, after performing the lysing step, the method includes adding barcoding sequences to the isolated DNA or RNA fragments to create a barcoded indexed library. In some embodiments the set of indexing primers include barcoding sequences. In certain embodiments, barcode sequences are added to the DNA or RNA fragments to allow for identification of specific cell phenotypes from which amplified nucleic acids originated. In some embodiments, barcodes may be added at one or both ends of each DNA or RNA fragment. The term “barcode”, as used herein and in its conventional sense, refers to a nucleic acid sequence that is used to identify a single cell or a subpopulation of cells. Barcode sequences can be linked to a target nucleic acid of interest during amplification or ligation and used to trace back the amplicon or ligated DNA or RNA fragment to the cell or population of cells from which the target nucleic acid originated. A barcode sequence can be added to a target nucleic acid of interest during amplification or ligation by carrying out PCR or ligation with a with the barcode sequence such that the barcode sequence is incorporated into the final amplified or ligated target nucleic acid product.

In some embodiments, after performing the lysing step, the method includes ligating the DNA or RNA fragments with barcode adapter sequences. In certain embodiments, the barcode adapter sequences comprise a set of forward and/or reverse barcoding adapter sequences. In some embodiments, ligating the forward and/or reverse barcode adapter sequences occurs before sorting, after sorting but before the purifying step, or after the purifying in step.

In other embodiments, after performing the lysing step, the method includes contacting the DNA or RNA fragments with a set of forward and/or reverse barcoding primers, and amplifying the DNA or RNA fragments to produce a barcoded indexed library.

### Cell Sorting

The ligation-based method or amplicon-based method of the present application can include additional steps such as antibody staining and/or cell sorting

In some embodiments, contacting the cells with an antibody or detectable molecule recognizing DNA, RNA, protein, or other molecule can occur after the ligation step or after amplification in a amplicon-based method. In some embodiments, contacting the cells with an antibody or detectable molecule recognizing DNA, RNA, protein, or other molecule can occur before the enzymatic fragmentation step. In some embodiments, contacting the cells with an antibody or detectable molecule recognizing DNA, RNA, protein, or other molecule can occur after an in situ PCR step.

In some embodiments, the ligation-based method includes sorting the cell/nucleic population into subpopulations by phenotypes (ie combinations of detectable molecules) to determine target cells/nuclei and non-target cells/nuclei. In certain embodiments, the sorting occurs after the ligation step. In certain embodiments, the sorting occurs after an in situ PCR step. Cell sorting and/or detectable labels facilitates the differentiation of cells by cell size, granularity, DNA content, morphology, differential protein expression (e.g., presence or absence of protein expression, or an amount of protein expression), calcium flux, and the like.

Aspects of the amplicon-based library preparation method of the present disclosure include after the first amplification step (e.g., target amplification), and/or after the second amplification step (e.g., adding adapter sequences), the method optionally includes antibody staining and sorting the cell/nuclei population into subpopulations by phenotypes to determine target cells/nuclei and non-target cells/nuclei.

In some embodiments, sorting the cells or contacting the cells with one or more detectable label provides for sorting protein-expressing cells, cells that secrete proteins, cells expressing an antigen-specific antibody, and the like. In some embodiments, before sorting, the cell/nuclei population is contacted with an antibody being directed against a distinct cell surface molecule on the cell, under conditions effective to allow antibody binding. In some embodiments, cell sorting and/or contacting the sample with a detectable label provides for differentiating cells by morphology presence or absence of chromatin (e.g., clumped chromatin), or the absence of conspicuous nucleoli.

In some embodiments, the cell/nuclei population can be prepared to include a detectable label, e.g., aptamers, cell stains, etc. For example, the cell/nuclei population can be prepared by adding one or more primary and/or secondary antibodies to the sample. Primary antibodies can include antibodies specific for a particular cell type or cell surface molecule on a cell. Secondary antibodies can include detectable labels (e.g., fluorescence label) that bind to the primary antibody. Additional non-limiting examples of detectable labels include: Haematoxylin and Eosin staining, Acid and Basic Fuchsin Stain, Wright's Stain, antibody staining, cell membrane fluorescent dye, carboxyfluorescein succinimidyl ester (CFSE), DNA stains, cell viability dyes such as DAPI, PI, 7-AAD, fixable compatible dyes, amine dyes, and the like.

In some embodiments, primary antibodies are added to the sample containing the cell/nuclei population before enzymatic fragmentation. In some embodiments, primary antibodies are added to the sample containing the cell/nuclei population the lysing step of lysing the cells.

In some embodiments, primary and secondary antibodies are added to the sample before the lysing step of lysing the cells. In other embodiments, primary antibodies are added to the sample before the enzymatic fragmentation step, and the secondary antibody or detectable label are added to the sample before the lysing step of lysing the cells.

Non-limiting examples of cell sorting techniques that can be used in the present methods include, but are not limited to, flow cytometry, fluorescence activated cell sorting (FACS), in situ hybridization (ISH), fluorescence in situ hybridization, Ramen flow cytometry, fluorescence microscopy, optical tweezers, micro-pipettes, microfluidic magnetic separation devices, and magnetic activated cell sorting, and methods thereof. In some embodiments, the sorting step of the methods of the present disclosure includes FACS techniques, where FACS is used to select cells from the population containing a particular surface marker, or the selection step can include the use of magnetically responsive particles as retrievable supports for target cell capture and/or background removal. For example, a variety FACS systems are known in the art and can be used in the methods of the invention (see e.g., PCT Application Publication No.: WO99/54494, US Application No. 20010006787, U.S. Pat. No. 10,161,007, each expressly incorporated herein by reference in their entirety).

In some embodiments, sorting comprises sorting the cell/nuclei population having a plurality of phenotypes into subpopulations by phenotypes to determine target cells/nuclei and non-target cells/nuclei within the population. In some embodiments, cells are sorted into subpopulations of cells irrespective of phenotypes.

### 6.1.1.7 Purifying the Ligated DNA or RNA Fragments

In some aspects, the ligation-based method of the present application includes purifying the ligated DNA or RNA fragments of the cells/nuclei. Purification of the ligated DNA or RNA fragments can be performed after lysing the cells, but before sequencing. For example, the purification step can be performed after any one of the following steps: after ligation and lysing; after ligation, one or more additional PCR steps and lysing; after ligation-based or amplification-based barcoding and lysing; or after ligation, cell sorting, and lysing the cells.

Techniques for purifying ligated DNA or RNA fragments are well-known in the art and include, for example, using size selection based magnetic bead purification reagent (e.g., Solid Phase Reversible Immobilization (SPRI) beads) passing through a column, phenol chloroform and the like. In some embodiments, purifying ligated DNA or RNA fragments can include using magnetic streptavidin beads, for example if the DNA or RNA fragments contain biotin.

In some embodiments, purifying the ligated DNA or RNA fragments of the present methods creates an enriched or purified library for sequencing. The term “enriched” as used herein and in its conventional sense, refers to isolated nucleotide sequences containing the genomic regions of interest (e.g., target regions) using known purification techniques (e.g., hybridization capture, magnetic bead purification techniques, and the like). The purified libraries described in the methods herein includes the final purified library before sequencing.

In some embodiments, the purifying step includes bead purification techniques using one or more of the following techniques: a bead-based size selection (e.g., AMPure), column based PCR cleanup (e.g., Qiagen), or a DNA precipitation bases technique using phenyoll chloroform.

In some embodiments, the ligation-based method includes performing additional amplification/PCR and/or ligation steps after purification.

### Hybridization Capture

In some embodiments, the ligation-based method includes performing hybridization capture on the purified library. For example, this step can occur before sequencing.

This purified library may optionally contain barcoded sequences ligated or amplified onto the DNA or RNA fragments.

Hybridization capture can be performed using any conventionally acceptable hybridization capture technique. For example, in one embodiment, performing hybridization capture comprises contacting the purified library (e.g., purified library with or without barcode sequences) with oligonucleotides configured to hybridize to one or more target DNA or RNA sequences and performing hybridization capture on purified DNA or RNA fragments.

In some embodiments, hybridization capture protocols described herein can include DNA from 1 cell population per hybridization capture reaction, or 2 or more, 3 or more, 4 or more, 5 or more, 6 or more, 7 or more, 8 or more, 9 or more, 10 or more, 15 or more, 20 or more, 25 or more, 30 or more, 35 or more, 40 or more, 45 or more, or 50 or more pooled populations per hybridization capture reaction. Non-limiting examples of kits that can be used for the hybridization capture methods can include but are not limited to Agilent SureSelectXT2, Twist Fast Hybridization and Wash Kit, Roche KAPA Target Enrichment, and the like.

In some embodiments, performing hybridization capture includes hybridizing the purified DNA or RNA fragments of the purified library with oligonucleotides to produce the enriched nucleic acid library. In some embodiments, performing hybridization capture includes contacting the purified DNA or RNA fragments with a one or more oligonucleotides that hybridize to target purified DNA or RNA fragments.

In such embodiments, the method further includes hybridizing blocking oligonucleotides in the same hybridization reaction. In certain embodiments, the blocking oligonucleotides are xGen Universal Blockers.

In some embodiments, the one or more oligonucleotides comprises a set of oligonucleotides that are biotinylated.

In some embodiments, hybridization capture further comprises adding magnetic streptavidin beads that bind to the one or more oligonucleotide probes. In some embodiments, after the oligonucleotide probes are captured using magnetic streptavidin bead, the captured/enriched amplicon product is eluted and amplified another time.

In some embodiments, hybridization capture occurs in solution or on a solid support.

A non-limiting example of a hybridization capture method includes hybridizing oligonucleotide probes to the purified DNA or RNA fragments. Oligonucleotide probes can be DNA or RNA, and can be double-stranded, or single-stranded. In some embodiments, the oligonucleotides have biotinylated nucleotides incorporated into the oligonucleotides. Hybridization typically occurs by repeatedly heating and cooling the sample to increase association of the probe to the DNA or RNA. In some embodiments, oligonucleotide blockers are added to reduce likelihood of over-represented genomic sequences from mis-associating with the probes and also prevent the adapters attached to the PCR DNA or RNA fragments from binding to each other or genomic sequences. After hybridization, the probes are captured using magnetic streptavidin bead (via strong association with the biotin on the probe), then the “captured” Pre-Cap PCR product (e.g., purified DNA or RNA fragments) is eluted and amplified.

In some embodiments, after hybridization capture, the method includes eluting the purified DNA or RNA fragment. In some embodiments, the method includes amplifying the eluted captured/enriched purified DNA or RNA fragment.

In some embodiments, the oligonucleotides are designed to hybridize to multiple targets with the use of multiple oligonucleotides in a single hybridization capture experiment.

In some embodiments, the oligonucleotides are DNA oligonucleotides. In some embodiments, the oligonucleotides are RNA oligonucleotides. In some embodiments, the oligonucleotides are single stranded. In some embodiments, the oligonucleotides are double stranded.

In some embodiments, capture oligonucleotides are used during the hybridization capture method. For example, capture oligonucleotides are biotinylated oligonucleotide baits. Oligonucleotide biotinylated baits are designed to hybridize to regions of interest (e.g., target regions). In certain embodiments, after hybridization of oligonucleotide baits to the target regions, contacting the hybridized oligonucleotide baits with streptavidin beads to separate the bait:target nucleic acid complex from other fragments that are not bound to baits.

In some embodiments, each oligonucleotide comprises a nucleotide sequence that hybridize to an anti-sense strand of a nucleotide sequence encoding a target region of one or more cells. In some embodiments, each oligonucleotide comprises a unique nucleotide sequence that hybridizes to an anti-sense strand of a nucleotide sequence encoding a different target region of one or more cells. Thus, an oligonucleotide pool can include a plurality of oligonucleotides, where each oligonucleotide hybridizes to a distinct target nucleic acid.

In embodiments where hybrid capture is performed, an oligonucleotide pool includes oligonucleotides of a xGen Lockdown Panel. In certain embodiments where hybrid capture is performed, a oligonucleotide pool includes oligonucleotides of a xGen Probe Pool. In certain embodiments where hybrid capture is performed, a oligonucleotide pool includes oligonucleotides of a xGen lockdown Panels and Probe Pools. In certain embodiments where hybrid capture is performed, a oligonucleotide pool includes oligonucleotides of a xGen lockdown Panels and Probe Pools. In some embodiments, the panels comprise probes to target genes associated with a disease or condition. In some embodiments, the target genes are selected from one or more of: PD-L1, PD-1, HER2, BL1, CCDC6, EIF1AX, HIST1H2BD, MED12, POLE, SMARCB1, UPF3A, ACO1, CCND1, EIF2S2, HIST1H3B, MED23, POT1, SMC1A, VHL, ACVR1, CD1D, ELF3, HIST1H4E, MEN1, POU2AF1, SMC3, WASF3, ACVR1B, CD58, EML4, HLA-A, MET, POU2F2, SMO, WT1, ACVR2A, CD70, EP300, HLA-B, MGA, PPM1D, SMTNL2, XIRP2, ACVR2B, CD79A, EPAS1, HLA-C, MLH1, PPP2R1A, SNX25, XPO1, ADNP, CD79B, EPHA2, HNF1A, MPL, PPP6C, SOCS1, ZBTB20, AJUBA, CDC27, EPS8, HOXB3, MPO, PRDM1, SOX17, ZBTB7B, AKT1, CDC73, ERBB2, HRAS, MSH2, PRKAR1A, SOX9, ZFHX3, ALB, CDH1, ERBB3, IDH1, MSH6, PSG4, SPEN, ZFP36L1, ALK, CDH10, ERCC2, IDH2, MTOR, PSIP1, SPOP, ZFP36L2, ALPK2, CDK12, ERG, IKBKB, MUC17, PTCH1, SPTAN1, ZFX, AMER1, CDK4, ESR1, IKZF1, MUC6, PTEN, SRC, ZMYM3, APC, CDKN1A, ETNK1, IL6ST, MXRA5, PTPN11, SRSF2, ZNF471, APOL2, CDKN1B, EZH2, IL7R, MYD88, PTPRB, STAG2, ZNF620, ARHGAP35, CDKN2A, FAM104A, ING1, MYOCD, QKI, STAT3, ZNF750, ARHGAP5, CDKN2C, FAM166A, INTS12, MYOD1, RAC1, STAT5B, ZNF800, ARID1A, CEBPA, FAM46C, IPO7, NBPF1, RACGAP1, STK11, ZNRF3, ARID1B, CHD4, FAT1, IRF4, NCOR1, RAD21, STK19, ZRSR2, ARID2, CHD8, FBXO11, ITGB7, NF1, RASA1, STX2, ARID5B, CIB3, FBXW7, ITPKB, NF2, RB1, SUFU, ASXL1, CIC, FGFR1, JAK1, NFE2L2, RBM10, TBC1D12, ATM, CMTR2, FGFR2, JAK2, NIPBL, RET, TBL1XR1, ATP1A1, CNBD1, FGFR3, JAK3, NOTCH1, RHEB, TBX3, ATP1B1, CNOT3, FLG, KANSL1, NOTCH2, RHOA, TCEB1, ATP2B3, COL2A1, FLT3, KCNJ5, NPM1, RHOB, TCF12, ATRX, COL5A1, FOSL2, KDM5C, NRAS, RIT1, TCF7L2, AXIN1, COL5A3, FOXA1, KDM6A, NSD1, RNF43, TCP11L2, AXIN2, CREBBP, FOXA2, KDR, NT5C2, RPL10, TDRD10, AZGP1, CRLF2, FOXL2, KEAP1, NTN4, RPL22, TERT, B2M, CSDE1, FOXQ1, KEL, NTRK3, RPL5, TET2, BAP1, CSF1R, FRMD7, KIT, NUP210L, RPS15, TG, BCLAF1, CSF3R, FUBP1, KLF4, OMA1, RPS2, TGFBR2, BCOR, CTCF, GAGE12J, KLF5, OR4A16, RPS6KA3, TGIF1, BHMT2, CTNNA1, GATA1, KLHL8, OR4N2, RREB1, TIMM17A, BIRC3, CTNNB1, GATA2, KMT2A, OR52N1, RUNX1, TNF, BMPR2, CUL3, GATA3, KMT2B, OTUD7A, RXRA, TNFAIP3, BRAF, CUL4B, GNAll, KMT2C, PAPD5, SELP, TNFRSF14, BRCA1, CUX1, GNA13, KMT2D, PAX5, SETBP1, TOP2A, BRCA2, CYLD, GNAQ, KRAS, PBRM1, SETD2, TP53, BRD7, DAXX, GNAS, KRT5, PCBP1, SF3B1, TRAF3, C3orf70, DDX3X, GNB1, LATS2, PDAP1, SGK1, TRAF7, CACNA1D, DDX5, GNPTAB, LCTL, PDGFRA, SH2B3, TRIM23, CALR, DIAPH1, GPS2, LZTR1, PDSS2, SLC1A3, TSC1, CARD11, DICER1, GTF2I, MAP2K1, PDYN, SLC26A3, TSC2, CASP8, DIS3, GUSB, MAP2K2, PHF6, SLC44A3, TSHR, CBFB, DNM2, H3F3A, MAP2K4, PHOX2B, SLC4A5, TTLL9, CBL, DNMT3A, H3F3B, MAP2K7, PIK3CA, SMAD2, TYRO3, CBLB, EEF1A1, HIST1H1C, MAP3K1, PIK3R1, SMAD4, U2AF1, CCDCl20, EGFR, HIST1H1E, MAX, PLCG1, SMARCA4, and UBR5.

### 6.1.1.8 Sequencing of Nucleic Acids

Aspects of the present methods include sequencing the purified libraries. Sequencing occurs after the purification step; after the purification and additional ligation/PCR steps; or after the purification and additional ligation/PCR and hybridization capture steps.

Any high-throughput technique for sequencing can be used in the practice of the methods described herein. For example, DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like. These sequencing approaches can thus be used to sequence target nucleic acids of interest, for example, nucleic acids encoding target genes and other phenotypic markers amplified from the cell/nuclei populations.

In some embodiments, sequencing comprises whole genome sequencing.

Certain high-throughput methods of sequencing comprise a step in which individual molecules are spatially isolated on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLID sequencing or polony sequencing, e.g. Kim et al, Science, 316: 1481-1414 (2007)). Such methods may comprise amplifying the isolated molecules either before or after they are spatially isolated on a solid surface. Prior amplification may comprise emulsion-based amplification, such as emulsion PCR, or rolling circle amplification.

In some embodiments, sequencing is performed on the Illumina® MiSeq platform, which uses reversible-terminator sequencing by synthesis technology (see, e.g., Shen et al. (2012) BMC Bioinformatics 13:160; Junemann et al. (2013) Nat. Biotechnol. 31(4):294-296; Glenn (2011) Mol. Ecol. Resour. 11(5):759-769; Thudi et al. (2012) Brief Funct. Genomics 11(1):3-11; herein incorporated by reference in its entirety), NovaSeq, NextSeq, HiSeq, and the like. In some embodiments, sequencing is performed on any preferred, standard sequencing platform.

Aspects of the present methods include sequencing target nucleic acids of interest, for example, nucleic acids encoding target genes and other phenotypic markers amplified from the one or more cell populations.

### 6.1.2 Amplicon-Based Library In Situ

Aspects of the present disclosure include amplicon-based library preparation methods.

In one aspect, the amplicon-based library in situ preparation includes (a) providing a sample comprising a cell population; (b) amplifying, in each cell within the cell population, DNA or RNA with a primer pool set to produce a first set of amplicon products for each cell; (c) lysing each of the cells to isolate DNA or RNA fragments within the first set of amplicon products; (d) purifying the DNA or RNA fragments of the cells; and (e) sequencing the DNA or RNA fragments of the cells.

6.1.2.1 Amplification of DNA or RNA to Produce First Set of Amplicon Products

In the amplicon-based library preparation method, the method includes amplifying, in each cell within the cell/nuclei population, DNA or RNA with a primer pool set to produce a first set of amplicon products for each cell.

In some embodiments, the primers in the primer pool set are DNA primers. In some embodiments, the primers in the primer pool set are RNA primers. In some embodiments, the primer pool set includes targeting primers for targeting the target sequence region of the DNA or RNA within the cell/nuclei population.

### Primer Sets

In some embodiments, the first primer pool set of the present disclosure is designed to amplify multiple targets with the use of multiple primer pairs in a PCR experiment (e.g. in 1 or more PCR steps, 2 or more PCR steps, or 3 or more PCR steps).

In some embodiments the first primer pool set comprises a first forward primer pool. In some embodiments, the first primer pool set comprises a first reverse primer pool. The number primers within each primer pool set is dependent on the number of targets that will be prepared using the amplicon-based method. In some embodiments, the primers in the primer pool set further comprises indexing primers (e.g. barcoding primers).

In some embodiments the primer pool set comprises a first forward primer pool and a reverse primer pool. In some embodiments, the first primer pool set comprises 5 or more, 10 or more, 15 or more, 20 or more, 25 or more, 30 or more, 35 or more, 40 or more, 45 or more, 50 or more, 55 or more, 60 or more, 70 or more, 80 or more, 90 or more, or 100 or more forward and/or reverse primers. In some embodiments, the first primer pool set comprises 100 or more, 125 or more, 150 or more, 175 or more, 200 or more, 225 or more, 250 or more, 275 or more, 300 or more, 325 or more, 350 or more, 375 or more, 400 or more, 425 or more, 450 or more, 475 or more, or 500 or more forward and/or reverse primers. In some embodiments, the first primer pool set includes a range of 5-1000 forward and/or reverse primers. In some embodiments, the first primer pool set includes a range of 5-25, 25 to 50, 50 to 75, 75 to 100, 100 to 150, 150 to 200, 200 to 250, 250 to 300, 300 to 350, 350 to 400, 400 to 450, 450 to 500, 500 to 550, 550 to 600, 600 to 650, 650 to 700, 700 to 750, 750 to 800, 800 to 850, 850 to 900, 900 to 950, or 950 to 1000 forward and/or reverse primers. In some embodiments, the first primer pool set includes 1000 or more, 1500 or more, 2000 or more, 2500 or more, 3000 or more, 3500 or more, 4000 or more, 4500 or more, 5000 or more, 5500 or more, 6000 or more, 6500 or more, 7000 or more, 7500 or more, 8000 or more, 8500 or more, 9000 or more, 9500 or more, 10,000 or more, 10,500 or more, 11,000 or more, 11,500 or more, 12,000 or more, 12,500 or more, 13,000 or more, 13,500 or more, 14,000 or more, 14,500 or more, 15,000 or more, 15,500 or more, 20,000 or more, 20,500 or more, 21,500 or more, 22,000 or more, 22,500 or more, 23,000 or more, 24,500 or more, 25,000 or more, 25,500 or more, 26,000 or more, 26,500 or more, 27,000 or more, 27,500 or more, 28,000 or more, 28,500 or more, or 30,000 or more forward and/or reverse primers. In some embodiments, the first primer pool set includes 25,000 or more, 30,000 or more, 35,000 or more, 40,000 or more, 45,000 or more, 50,000 or more, 55,000 or more, 60,000 or more, or 65,000 or more forward and/or reverse primers. In some embodiments, the first primer pool set ranges from 1-30,000 forward and/or reverse primers, 1-60,000 forward and/or reverse primers, 1-50,000 forward and/or reverse primers, 1-25,000 forward and/or reverse primers, 1-26,000 forward and/or reverse primers, 1-1000 forward and/or reverse primers, 1000-2000 forward and/or reverse primers, 2000-3000 forward and/or reverse primers, 3000-4000 forward and/or reverse primers, 4000-5000 forward and/or reverse primers, 5000-6000 forward and/or reverse primers, 6000-7000 forward and/or reverse primers, 7000-8000 forward and/or reverse primers, 8000-9000 forward and/or reverse primers, 9000 to 10,000 forward and/or reverse primers, 10,000 to 11,000 forward and/or reverse primers, 11,000 to 12,000 forward and/or reverse primers, 12,000 to 13,000 forward and/or reverse primers, 13,000 to 14,000 forward and/or reverse primers, 14,000 to 15,000 forward and/or reverse primers, 15,000 to 16,000 forward and/or reverse primers, 16,000 to 17,000 forward and/or reverse primers, 17,000 to 18,000 forward and/or reverse primers, 18,000 to 19,000 forward and/or reverse primers, 19,000 to 20,000 forward and/or reverse primers, 20,000 to 21,000 forward and/or reverse primers, 21,000 to 22,000 forward and/or reverse primers, 22,000 to 23,000 forward and/or reverse primers, 23,000 to 24,000 forward and/or reverse primers, 24,000 to 25,000 forward and/or reverse primers, 25,000 to 26,000 forward and/or reverse primers, 26,000 to 27,000 forward and/or reverse primers, 27,000 to 28,000 forward and/or reverse primers, 28,000 to 29,000 forward and/or reverse primers, 29,000 to 30,000 forward and/or reverse primers, 30,000 to 40,000 forward and/or reverse primers, 40,000 to 50,000 forward and/or reverse primers, or 50,000 to 60,000 forward and/or reverse primers.

In some embodiments, each forward primer and each reverse primer includes a nucleotide sequence having a length ranging from 10 to 200 nucleotides; such as, 10 to 20 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, 140 to 150 nucleotides, 150 to 160 nucleotides, 160 to 170 nucleotides, 170 to 180 nucleotides, 180 to 190 nucleotides, or 190 to 200 nucleotides. In some embodiments, each forward and each reverse primer includes a nucleotide sequence having a length ranging from 10 to 50 nucleotides, such as 10 to 30, 20 to 40, or 30 to 50 nucleotides. In some embodiments, each forward and each reverse primer includes a nucleotide sequence having a length ranging from 10 to 20 nucleotides, such as 10 to 12, 12 to 14, 10 to 15, 14 to 16, 16 to 18, or 18 to 20 nucleotides. In some embodiments, each forward and each reverse primer includes a nucleotide sequence having a length of 10, 11, 12, 13, 14, 15, 16, 17, 18, 19, 20, 21, 22, 23, 24, 25, 26, 27, 28, 29, or 30 nucleotides. Forward primers within the set of forward primers can have different lengths. Similarly, reverse primers within the set of reverse primers can have different lengths. In certain embodiments, forward primers within the set of forward primers can have different lengths but similar Melting Temperature (Tm) and thus can have similar PCR reaction times. Reverse primers within the set of reverse primers can have different lengths but similar Melting Temperature (Tm) and thus can have similar PCR reaction times.

In some embodiments, each forward primer comprises a nucleotide sequence that hybridize to an anti-sense strand of a nucleotide sequence encoding a target region (e.g., target region of the DNA or RNA) of one or more cells. In some embodiments, the nucleotide sequence is a DNA sequence. In some embodiments, the nucleotide sequence is an RNA sequence. In some embodiments, each primer comprises a unique nucleotide sequence that hybridizes to an anti-sense strand of a nucleotide sequence encoding a different target region (e.g., a different target region of the DNA or RNA) of one or more cells. Thus, a forward primer pool can include a plurality of forward primers, where each forward primer hybridizes to a distinct target nucleic acid.

In some embodiments, each reverse primer comprises a nucleotide sequence that hybridize to a sense strand of a nucleotide sequence encoding a target region of one or more cells. In some embodiments, each primer comprises a unique nucleotide sequence that hybridizes to an anti-sense strand of a nucleotide sequence encoding a different target region of one or more cells. Thus, a reverse primer pool can include a plurality of reverse primers, where each reverse primer hybridizes to a distinct target nucleic acid. In some embodiments, the primers can include a modification that is cleaved off before they are able to polymerize.

As described herein, a first primer pool set can include publicly available primer pool sets of known nucleic target regions of interest. In some embodiments, the first primer pool set can include any standard multiplexing primer panel for sequencing. In some embodiments, a forward primer pool includes primers selected from a rhAmp PCR Panel, CleanPlex® NGS Panel, and Ampliseq Panel. In some embodiments, a reverse primer pool includes primers of a rhAmp PCR Panel, CleanPlex® NGS Panel, and Ampliseq Panel. However, the forward and revers primers do not need to be from any existing panels. In some embodiments, the primer pool set comprises RNA:DNA hybrids. In some embodiments the panel includes only the target regions of interest. In some embodiments the panel includes both the target region of interest and a common sequence, such that the target region of interest is on the 3′ end of the common sequence.

Aspects of the present disclosure include amplifying the DNA or RNA within the cell/nuclei population using the first primer pool set to produce a first set of amplicon products. In some embodiments, the nucleic acids of the cell/nuclei population are amplified in situ.

The term “amplicon”, as used herein and in its conventional sense, refers to the amplified nucleic acid product of a PCR reaction or other nucleic acid amplification process (e.g., ligase chain reaction (LGR), nucleic acid sequence-based amplification (NASBA), transcription-mediated amplification (TMA), Q-beta amplification, strand displacement amplification, target mediated amplification, and the like). Amplicons may comprise RNA or DNA depending on the technique used for amplification. For example, DNA amplicons may be generated by RT-PCR, whereas RNA amplicons may be generated by TMA/NASBA.

### Multiplexed Polymerase Chain Reaction

As explained above, the primer sets described herein by are used in in situ PCR—for amplification of target nucleic acids in a sample containing a cell/nuclei population. PCR is a technique for amplifying desired target nucleic acid sequence contained in a nucleic acid molecule or mixture of molecules. In PCR, a pair of primers is employed in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, N Y 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

The present methods can use PCR for amplification of DNA or RNA fragments in one or more PCR reactions, with one or more of the PCR steps occurring in situ. As a non-limiting example, in a multiplexing assay, more than one target sequence can be amplified by using multiple primer pairs in a reaction mixture. PCR steps can also be used to create copies of amplicon products containing the DNA or RNA products. In some embodiments, multiple PCR reactions are performed between the first amplification step (e.g., target amplification) and the sequencing steps.

In particular, PCR uses relatively short oligonucleotide primers which flank the target nucleotide sequence to be amplified, oriented such that their 3′ ends face each other, each primer extending toward the other. The polynucleotide sample is extracted and denatured, e.g., by heat, and hybridized with first and second primers that are present in molar excess. Polymerization is catalyzed in the presence of the four deoxyribonucleotide triphosphates (dNTPs—dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. In some cases, PCR is carried out with a commercially available thermal cycler, e.g., Perkin Elmer.

RNA may be amplified by reverse transcribing the RNA into cDNA (RT-PCR) using an RNA dependent DNA polymerase (RT-PCR) with a single targeting primer set to the anti-sense strand of RNA, oligo-dT primers, or random sequences, such as a random hexamer. PCR amplification can then occur with addition targeting primers as described above. Alternatively, a single enzyme may be used for both steps as described in U.S. Pat. No. 5,322,770, incorporated herein by reference in its entirety. RNA may also be reverse transcribed into cDNA, followed by asymmetric gap ligase chain reaction (RT-AGLCR) as described by Marshall et al. (1994) PCR Meth. App. 4:80-84. Suitable DNA polymerases include reverse transcriptases, such as avian myeloblastosis virus (AMV) reverse transcriptase (available from, e.g., Seikagaku America, Inc.) and Moloney murine leukemia virus (MMLV) reverse transcriptase (available from, e.g., Bethesda Research Laboratories).

Any PCR reaction mixture (e.g., used interchangeably herein as “PCR Enzyme Master Mix”) and heat-resistant DNA polymerase may be used to produce amplicon products. For example, those contained in a commercially available PCR kit can be used. In some embodiments, the PCR reaction mixture can include other enzymes that aid in transcription (e.g., such as RNAseH to cleave a modification in primers). Non-limiting examples of a PCR kit includes rhAmpSeq Library Kit (IDT) and rhAmpSeq Library Mix. In some embodiments, one or more components of a PCR kit can be used in the PCR reaction mixture, at various concentrations. As the reaction mixture, any buffer known to be usually used for PCR can be used. Examples include IDTE (10 mM Tris, 0.1 mM EDTA; Integrated DNA Technologies), Tris-HCl buffer, a Tris-sulfuric acid buffer, a tricine buffer, and the like. Examples of heat-resistant polymerases include Taq DNA polymerase (e.g., FastStart Taq DNA Polymerase (Roche), Ex Taq (registered trademark) (Takara), Z-Taq, AccuPrime Taq DNA Polymerase, M-PCR kit (QIAGEN), KOD DNA polymerase, and the like.

The amounts of the primer and template DNA used, etc., in the present disclosure can be adjusted according to the PCR kit and device used. In some embodiments, about 0.1 to 1 μl of the first primer pool set is added to the in situ PCR reaction mixture. In some embodiments, a forward primer pool of about 0.5 μl, about 1 μl, about 1.5 μl, about 2 μl, about 2.5 μl, about 3 μl, about 3.5 μl, about 4 μl, about 4.5 μl, or about 5 μl is added to the PCR reaction mixture. In some embodiments, a reverse primer pool of about 0.5 μl, about 1 μl, about 1.5 μl, about 2 μl, about 2.5 μl, about 3 μl, about 3.5 μl, about 4 μl, about 4.5 μl, or about 5 μl is added to the PCR reaction mixture.

In some embodiments, the PCR reaction mixture includes the first primer pool set, the population of cells, and a PCR library mix. Any standard PCR library mix can be used in the PCR reaction mixture. In some embodiments, the library mix is a rhAmpSeq Library Mix or components of the rhAmpSeq Library Mix. In some embodiments, the PCR library mix contains one or more components of a rhAmpSeq Library mix or one or more components of any standard PCR Library mixture. In some embodiments, a forward primer pool of the first primer pool set includes forward primers of a rhAmp PCR Panel. In some embodiments, a reverse primer pool of the first primer pool set includes reverse primers of a rhAmp PCR Panel. However, any standard PCR library mix or PCR Enzyme Master Mix for sequencing can be used.

In some embodiments, about 0.1 to 10 μl of the PCR library mix is added to the PCR reaction mixture. In some embodiments, a PCR library mix of about 0.5 μl, about 1 about 1.5 μl, about 2 μl, about 2.5 μl, about 3 μl, about 3.5 μl, about 4 μl, about 4.5 μl, about 5 μl, about 6 about 7 μl, about 8 μl, about 9 μl, or about 10 μl, is added to the PCR reaction mixture.

The PCR reaction mixture of the present disclosure includes one or more cell populations. In some embodiments, the cell population is diluted to a volume of about 0.5 μl, about 1 μl, about 1.5 μl, about 2 μl, about 2.5 μl, about 3 μl, about 3.5 μl, about 4 μl, about 4.5 μl, about 5 μl, about 6 μl, about 7 μl, about 8 μl, about 9 about 10 μl, about 11 μl, about 12 μl, about 13 μl, about 14 μl, about 15 μl, about 16 μl, about 17 μl, about 18 μl, about 19 or about 20 μl.

In some embodiments, the cell/nuclei population is diluted to contain 1 to 30,000 cells. In some embodiments, the cell/nuclei population is diluted to contain 1 to 20,000 cells. In some embodiments, the cell/nuclei population is diluted to contain 1 to 15,000 cells. In some embodiments, the cell/nuclei population is diluted to contain 1 to 16,000 cells. In some embodiments, the cell/nuclei population is diluted to contain 1 to 15,000 cells. In some embodiments, the cell/nuclei population is diluted to contain 1 to 10,000 cells. In some embodiments, the cell/nuclei population is diluted to contain 1 to 100 cells, 100 to 200 cells, 200 to 300 cells, 300 to 400 cells 400 to 500 cells, 500 to 600 cells, 600 to 700 cells, 700 to 800 cells, 800 to 900 cells, 900 to 1000 cells, 1000 to 1100 cells, 1100 to 1200 cells, 1200 to 1300 cells, 1300 to 1400 cells, or 1400 to 1500 cells. In some embodiments, the cell/nuclei population is diluted to contain 20,000 cells or less, 19,000 cells or less, 18,000 cells or less, 17,000 cells or less, 16,000 cells or less, 15,000 cells or less, 14,000 cells or less, 13,000 cells or less, 12,000 cells or less, 11,000 cells or less, 10,000 cells or less, 9,000 cells or less, 8,000 cells or less, 7,000 cells or less, 6,000 cells or less, 5,000 cells or less, 4,000 cells or less, 3,000 cells or less, 2,000 cells or less, 1,500 cells or less, 1,000 cells or less, 500 cells, 250 cells or less, 100 cells or less, 50 cells or less, 25 cells or less, 10 cells or less, 5 cells or less, or 2 cells or less. In some embodiments, the cell/nuclei population is diluted to contain 1 cell. In some embodiments, the cell/nuclei population is diluted to contain 1 to 15,000 cells.

As described herein, the PCR cycling conditions are not particularly limited as long as the desired target genes can be amplified. For example, the thermal denaturation temperature can be set to 92 to 100° C., e.g., 94 to 98° C. The thermal denaturation time can be set to, for example, 5 to 180 seconds, e.g., 10 to 130 seconds. The annealing temperature for hybridizing primers can be set to, for example, 55 to 80° C., e.g., 60 to 70° C. The annealing time can be set to, for example, 10 to 60 seconds, e.g., 10 to 20 seconds. The extension reaction temperature can be set to, for example, 55 to 80° C., e.g., 60 to 70° C. The elongation reaction time can be set to, for example, 4 to 15 minutes, e.g., 10 to 20 minutes. In some embodiments, the annealing and extension reaction can be performed under the same conditions. In some embodiments, the operation of combining thermal denaturation, annealing, and an elongation reaction is defined as one cycle. This cycle can be repeated until the required amounts of amplification products are obtained. For example, the number of cycles can be set to 30 to 40 times, e.g., about 30 to 35 times. In some embodiments, the number of cycles can be set to 5 to 10 cycles, 10 to 15 cycles, 15 to 20 cycles, 20 to 25 cycles, 25 to 30 cycles, 35 to 40 cycles, 45 to 50 cycles, or 55 to 60 cycles.

In the present disclosure, the “PCR cycling conditions” may include one of, any combination of, or all of the conditions with respect to the temperature and time of each thermal denaturation, annealing, and elongation reaction of PCR and the number of cycles. When PCR cycling conditions are set, the touchdown PCR method can be used in terms of inhibiting non-specific amplification. Touchdown PCR is a technique in which the first annealing temperature is set to a relatively high temperature and the annealing temperature is gradually reduced for each cycle, and, midway and thereafter, PCR is performed in the same manner as general PCR. Shuttle PCR may also be used in terms of inhibiting non-specific amplification. Shuttle PCR is a PCR in which annealing and extension reaction are performed at the same temperature.

Although different PCR cycling conditions can be used for each primer pair, it is preferable from the viewpoint of operation and efficiency that PCR cycling conditions are set in such a manner that the same PCR cycling conditions can be used for different primer pairs and the variation of PCR cycling conditions used to obtain necessary amplification products is minimized. The number of variations of PCR cycling conditions is preferably 10 or less, 5 or less, more preferably 4 or less, still more preferably 3 or less, even more preferably 2 or less, and even still more preferably 1. When the number of variations of PCR cycling conditions used to obtain all the necessary amplification products is reduced, PCRs using the same PCR cycling conditions can be simultaneously performed using one PCR device. Accordingly, the desired amplification products can be obtained in a short time using smaller amounts of resources.

In some embodiments, the method of the present disclosure includes, after producing the first set of amplicon products, purifying the first set of amplicon products. Techniques for purifying amplicon products are well-known in the art and include, for example, using magnetic bead purification reagent, passing through a column, use of ampure beads, phenol chloroform and the like.

6.1.2.2 Additional Exemplary Amplification or Ligation Steps

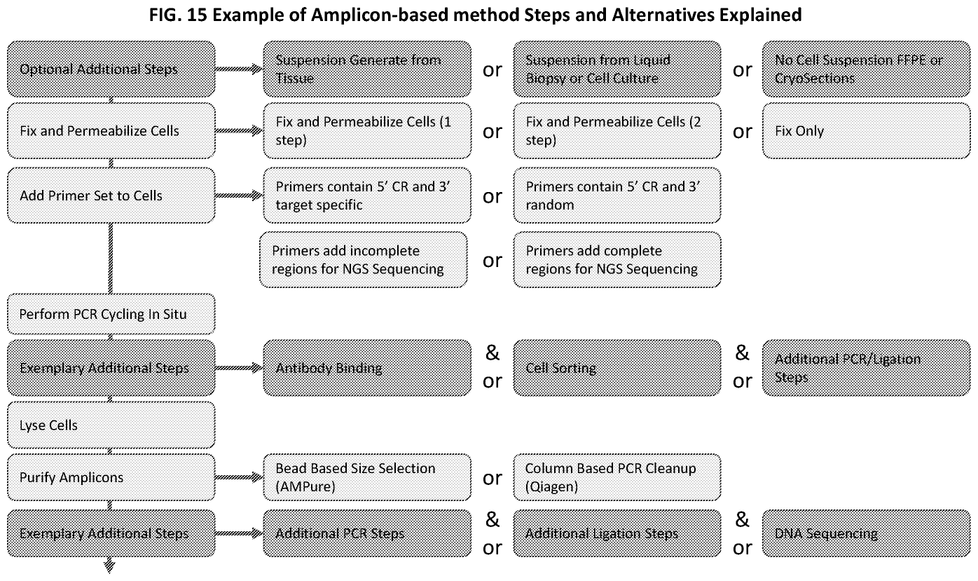

As described above and as shown in FIG. 15, the amplicon-based method of the present disclosure can include multiple additional PCR steps after the first amplification step and before sequencing. For example, additional PCR steps can be performed before or after lysing or after purification. The method can also include ligation steps to ligate on adapter sequences for subsequent PCR or direct sequencing.

In some embodiments, the method further comprises amplifying the first set of amplicon products with primer sequences to produce a set of amplicon products. This step can be performed after the first amplification step and before the lysing step, after the lysing step, or after a second amplification step (e.g., amplification with sample barcoding sequences). In some embodiments, the primer sequences include sample barcodes.

In some embodiments, the method further comprises, after the sorting step or lysing step, contacting the first set of amplicon products with sample barcoding sequences. In some embodiments, sample barcoding sequences comprise a set of forward and/or reverse sample barcoding primers, and wherein the method comprises amplifying the first set of amplicon products with the set of forward and/or reverse sample barcoding primers to produce a barcoded indexed library comprising sample barcoded amplicon products.

As an alternative to amplification of sample barcoding sequences, in some embodiments, the sample barcoding sequences comprise a set of barcoding adapters, and wherein the method comprises ligating the set of barcode adapters to produce a barcoded indexed library comprising barcoded amplicon products.

In some embodiments, the method further comprises ligating on adapter sequences. Non-limiting examples of adapter sequences include, but are not limited to, adapter nucleotide sequences that allow high-throughput sequencing of amplified nucleic acids. In some embodiments, the adapter sequences are selected from one or more of: a Y-adapter nucleotide sequence, a hairpin nucleotide sequence, a duplex nucleotide sequence, and the like. In some embodiments, the adapter sequences are for pair-end sequencing. In some embodiments, the adapter sequences include sequencing reads (e.g., R1, R2, etc.). In some embodiments, the adapter sequences include sample barcodes. Adapter sequences can be used in a ligation reaction of the disclosed method for the desired sequencing method used.

In some embodiments, ligating includes performing ligase chain reaction (LCR). The ligase chain reaction (LCR) is an amplification process that involves a thermostable ligase to join two probes or other molecules together. In some embodiments, the ligated product is then amplified to produce a second amplicon product. In some embodiments, LCR can be used as an alternative approach to PCR. In other embodiments, PCR can be performed after LCR.

In some embodiments, the thermostable ligase can include, but is not limited to Pfu ligase, or a Taq ligase. In some embodiments, the method further comprises, after the sorting step or lysing step, contacting the first set of amplicon products with sample barcoding sequences. In some embodiments, sample barcoding sequences comprise a set of forward and/or reverse sample barcoding primers, and wherein the method comprises amplifying the first set of amplicon products with the set of forward and/or reverse sample barcoding primers to produce a barcoded indexed library comprising sample barcoded amplicon products.

6.1.2.3 Additional Cell Sorting

Aspects of the amplicon-based library preparation method of the present disclosure include after the first amplification step (e.g., target amplification), and/or after the second amplification step (e.g., adding adapter sequences), the method optionally includes antibody staining and sorting the cell/nuclei population into subpopulations by phenotypes to determine target cells/nuclei and non-target cells/nuclei.

Cell sorting and/or detectable labeling of DNA or RNA fragments that can be performed in the amplicon-based library preparation method is previously described in section 6.1.1.6, under “cell sorting”.

6.1.2.4 Lysing the Cells to Collected DNA or RNA Fragments

The amplicon-based library preparation method of the present disclosure includes lysing each of the cells to isolate DNA or RNA fragments within the first set of amplicon products and has previously been described in section 6.1.1.5

The lysing step can be accomplished by contacting the DNA or RNA fragments within the cell with a cell lysing agent. In some embodiments, said lysing occurs after the ligation step. In some embodiments, lysing occurs after a sorting step. In some embodiments, lysing occurs after a PCR step (e.g. reaction). Lysing the cells with a cell lysing agent facilitates purification and isolation of the DNA or RNA fragments for each cell/nuclei population.

Non-limiting examples of cell lysing agents include, but are not limited to, an enzyme solution. In some embodiments, the enzyme solution includes a proteases or proteinase K, phenol and guanidine isothiocyanate, RNase inhibitors, SDS, sodium hydroxide, potassium acetate, and the like. However, any known cell lysis buffer may be used to lyse the cells within the one or more cell populations.

In some embodiments, lysing includes heating the cells for a period of time sufficient to lyse the cells. In certain embodiments, the cells can be heated to a temperature of about 80° C. or more, 85° C. or more, 90° C. or more, 96° C. or more, 97° C. or more, 98° C. or more, or 99° C. In certain embodiments, the cells can be heated to a temperature of about 90° C., 95° C., 96° C., 97° C., 98° C., or 99° C.

6.1.2.5 Purifying the Amplified DNA or RNA Fragments

In some aspects, the amplicon-based method of the present application includes purifying the amplicon products of the cells/nuclei. Purification of the amplicon products can be performed after lysing the cells, but before sequencing. For example, the purification step can be performed after any one of the following steps: after amplification and lysing; after amplification, one or more additional PCR or ligation steps and lysing; after ligation-based or amplification-based barcoding and lysing; or after amplification, cell sorting, and lysing the cells.

Techniques for purifying amplicon products are well-known in the art and include, for example, using magnetic bead purification reagent, passing through a column, use of ampure beads, and the like.

In some embodiments, purifying the amplicon products of the present methods creates an enriched or purified library for sequencing.

Purification techniques that can be performed in the amplicon-based methods of the present disclosure are previously described in section 6.1.1.7.

6.1.2.6 Sequencing of Nucleic Acids

Aspects of the present methods include sequencing the purified libraries. Sequencing occurs after the purification step; after the purification and additional ligation/PCR steps; or after the purification and additional PCR and/or ligation steps.

Any high-throughput technique for sequencing can be used in the practice of the methods described herein. For example, DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like. These sequencing approaches can thus be used to sequence target nucleic acids of interest, for example, nucleic acids encoding target genes and other phenotypic markers amplified from the one or more cell populations.

Various sequencing techniques that can be performed on the purified libraries are described in section “6.1.1.8”.

### 6.1.3 Additional Common Steps in Ligation-Based and Amplicon-Based Library Preparation Methods

Aspects of the ligation-based and amplicon-based library preparation methods of the present disclosure include additional steps that are common to both ligation-based and amplicon-based methods.

Fixing and/or Permeabilizing Cells

In some embodiments, the ligation-based or amplicon-based method includes, before providing a sample comprising a cell/nuclei population, fixing and/or permeabilizing the cell/nuclei population.

Fixing and/or permeabilizing cells from a cell/nuclei population can be performed upon collection of the sample.

In some embodiments, the method includes suspending one or more cells within cell/nuclei population in a liquid. In some embodiments, the cellular sample in suspension are fixed and permeabilized as desired.

Fixing and permeabilizing the cellular sample can be performed by any convenient method as desired. For example, in some embodiments, the cellular sample is fixed according to fixing and permeabilization techniques described in U.S. Pat. No. 10,627,389, which is hereby incorporated by reference in its entirety.

In some embodiments, fixing the cellular sample includes contacting the sample with a fixation reagent. Fixation reagents of interest are those that fix the cells at a desired time-point. Any convenient fixation reagent may be employed, where suitable fixation reagents include, but are not limited to: formaldehyde, paraformaldehyde, formaldehyde/acetone, methanol/acetone, IncellFP (IncellDx, Inc) etc. For example, paraformaldehyde used at a final concentration of about 1 to 15% has been found to be a good cross-linking fixative.

In some embodiments, the cells in the sample are permeabilized by contacting the cells with a permeabilizing reagent. Permeabilizing reagents of interest are reagents that allow the labeled biomarker probes, e.g., as described in greater detail below, to access to the intracellular environment. Any convenient permeabilizing reagent may be employed, where suitable reagents include, but are not limited to: mild detergents, such as EDTA, Tris, IDTE (10 mM Tris, 0.1 mM EDTA), Triton X-100, NP-40, saponin, Tween-20, etc.; methanol, and the like.

In some embodiments, a collected liquid sample, e.g., as obtained from fine needle aspirations (FNA) or a pipette that results in dissociation of the cells, is immediately contacted with solution intended to prepare the cells of the sample for further processing, e.g., fixation solution, permeabilization solution, staining solution, labeling solution, or combinations thereof, so to minimize degradation of the cells of the sample that may occur prior to preparation of the cells or prior to analysis of the cells. In some embodiments, the sample is frozen and thawed before processing. By “immediately contacted” used herein and in its conventional sense, the cells of the sample or the sample itself is contacted with the subject agent or solution without unnecessary delay from the time the sample is collected or thawed. In some embodiments, a sample is immediately contacted with a preparative agent or solution in 6 or less hours from the time the sample is collected, including but not limited to, e.g., 5 hours or less, 4 hours or less, 3 hours or less, 2 hours or less, 1 hours or less, 30 min. or less, 20 min. or less, 15 min. or less, 10 min. or less, 5 min. or less, 4 min. or less, 3 min. or less, 2 min. or less, 1 min. or less, etc., optionally including a lower limit of the minimum amount of time necessary to physically contact the sample with the preparative agent or solution, which may, in some instances be on the order of 1 sec. to 30 sec or more.

In some embodiments, the sample is a Formalin-Fixed Paraffin-Embedded (FFPE) tissue sample. In some embodiments, the sample is a cryopreserved tissue sample.

Aspects of the present methods include preparation of the sample and/or fixation of the cells of the sample performed in such a manner that the prepared cells of the sample maintain characteristics of the unprepared cells, including characteristics of unprepared cells in situ, i.e., prior to collection, and/or unfixed cells following collection but prior to fixation and/or permeabilization and/or labeling. Such characteristics that may be maintained include but are not limited to, e.g., cell morphological characteristics including but not limited to, e.g., cell size, cell volume, cell shape, etc. The preservation of cellular characteristics through sample preparation may be evaluated by any convenient means including, e.g., the comparison of prepared to cells to one or more control samples of cells such as unprepared or unfixed or unlabeled samples. Comparison of cells of a prepared sample to cells of an unprepared sample of a particular measured characteristic may provide a percent preservation of the characteristic that will vary depending on the particular characteristic evaluated. The percent preservation of cellular characteristics of cells prepared according to the methods described herein will vary and may range from 50% maintenance or more including but not limited to, e.g., 60% maintenance or more, 65% maintenance or more, 70% maintenance or more, 75% maintenance or more, 80% maintenance or more, 85% maintenance or more, 90% maintenance or more, etc., and optionally with a maximum of 100% maintenance. In some instances, preservation of a particular cellular characteristic may be evaluated based on comparison to a reference value of the characteristic (e.g., from a predetermined measurement of one or more control cells, from a known reference standard based on unprepared cells, etc.). In some embodiments, the cells may be evaluated using a hemocytometer, microscope, and/or any other known cell counting method.

In some embodiments, the method of fixing and permeabilizing the cells include spinning the cells down, contained within a tube, with a centrifuge (e.g., 1,000 G at 5 min) to separate the supernatant from the cells. In some embodiments, the method includes adding 500 μl freezing media after spinning the cells. In some embodiments, the cells in the freezing media are placed in a refrigerator at a temperature of about −20° C.±5° C. In some embodiments, the cells in the freezing media are placed in a refrigerator at a temperature of about −20° C.±10° C.

In such embodiments, the method includes removing the first supernatant without disturbing the cell pellet. In some embodiments, the method includes adding 100 μl IDTE buffer or any known permeabilizing buffer after removing the first supernatant.

In such embodiments, the method includes adding phosphate buffered saline (PBS) to the cells contained within the tube after removing the first supernatant. In some embodiments, the method includes adding 500 μl freezing media after adding PBS to the cells. In some embodiments, the cells in the freezing media are placed in a refrigerator at a temperature of about −20° C.±5° C. In some embodiments, the cells in the freezing media are placed in a refrigerator at a temperature of about −20° C.±10° C.

In such embodiments, the method includes gently mixing the cells after adding PBS by pipetting to re-suspend the cell pellet. In such embodiments, the method includes spinning the cells down (e.g., 300-1500 G at 5 min). In such embodiments, the method includes removing the second supernatant without disturbing the cell pellet. In some embodiments, the method includes adding PBS, IDTE or any known permeabilizing buffer to the cells. In some embodiments, about 11 μl of PBS is added to about 16,000 cells.

In some embodiments, the sample is a cell suspension generated from a tissue sample or a cell suspension generated from a liquid biopsy. In some embodiments the cell suspension is a crude suspension and suspended cells are not necessarily single cells. In some embodiments, the cell suspension comprises cell clusters of 2-100 cells, 2-500 cells, 2-1000 cells, 2-5000 cells, 2-10000 cells, and the like.

### Cell/Nuclei Population

The sample of the present ligation-based and amplicon-based methods include a cell/nuclei population (e.g., cell population and/or a cell nuclei population).

In some embodiments, the cell/nuclei population has a single phenotype. In some embodiments, the cell/nuclei population is a heterogenous cell/nuclei population having one or more distinct phenotypes. In some embodiments, the cell/nuclei population is a heterogenous cell/nuclei population having a plurality of phenotypes. In some embodiments, the cell/nuclei population is a heterogenous cell/nuclei population having a plurality of distinct phenotypes. In some embodiments, the cell/nuclei population is not a heterogeneous population. In some embodiments, the cell/nuclei population comprises one or more phenotypes, two or more phenotypes, three or more phenotypes, four or more phenotypes, five or more phenotypes, six or more phenotypes, seven or more phenotypes, eight or more phenotypes, nine or more phenotypes, or ten or more phenotypes. In some embodiments, the cell/nuclei population comprises multiple phenotypes. In some embodiments, the cell/nuclei population comprises a single phenotype. Non-limiting examples of phenotypes include, but are not limited to cell size, morphology, granularity, DNA content, protein expression, and the like.

In some embodiments, the cell/nuclei population can include one or more cell/nuclei populations and/or subcellular populations. In some embodiments, the cell/nuclei population are from a tumor biopsy. In some embodiments, the tumor sample is a solid tumor sample. In some embodiments, the tumor biopsy is a liquid tumor sample. In some embodiments, a tumor sample can include a heterogenous cell population. In some embodiments, the tumor sample is from human tumors such as, but not limited to, tumors from the breast, ovarian, lung, prostate, colon, renal, liver, skin blood, bone marrow, lymph nodes, spleen, thymus, etc. In some embodiments, cancer cells that can be detected by the methods of the present disclosure include, but are not limited to, cancer cells from hematological cancers, including leukemia, lymphoma and myeloma, and solid cancers, including for example tumors of the brain (glioblastomas, medulloblastoma, astrocytoma, oligodendroglioma, ependymomas), carcinomas, e.g. carcinoma of the lung, liver, thyroid, bone, adrenal, spleen, kidney, lymph node, small intestine, pancreas, colon, stomach, breast, endometrium, prostate, testicle, ovary, skin, head and neck, and esophagus.

Tumor microenvironments contain a heterogenous population of cells. Characterizing the composition and the interaction, dynamics, and function of a heterogenous population of cells at the single-cell resolution are important for fully understanding the biology of tumor heterogeneity, under both normal and diseased conditions. For example, cancer, a disease caused by somatic mutations conferring uncontrolled proliferation and invasiveness, can benefit from advances in single-cell analysis. Cancer cells can manifest resistance to various therapeutic drugs through cellular heterogeneity and plasticity. The tumor microenvironment includes an environment containing tumor cells that cooperate with other tumor cells and host cells in their microenvironment and can adapt and evolve to changing conditions.

In some embodiments, the heterogeneous population of cells can include, but are not limited to, inflammatory cells, cells that secret cytokines and/or chemokines, cytotoxic immune cells (e.g., natural killer and/or CD8+ T cells), immune cells, macrophages (e.g., immunosuppressive macrophages or tumor-associated macrophages), antigen-presenting cells, cancer cells, tumor-associated neutrophils, erythrocytes, dendritic cells (e.g., myeloid dendritic cells and/or plasmacytoid dendritic cells), B cells, tumor-infiltrated T cells, fibroblasts, endothelial cells, PD1+ T cells, and the like.

In some embodiments, the sample can be from cell lines such as ovarian cancer (A4, OVCAR3), teratocarcinoma (NT2), colon cancer (HT29), prostate (PC3, DU145), cervical cancer (ME180), kidney cancer (ACHN), lung cancer (A549), skin cancer (A431), glioma (C6), but are not limited to only these lines.

In some embodiments, the cell populations within the sample are from mutated/malignant tissue or abnormal blood. In some embodiments, the methods of the present disclosure steps are also performed on cell populations within the sample that are from non-mutated/benign tissue or normal blood, which serve as a controls sample. In some embodiments, the cell populations within the sample are from both non-mutated tissue or normal blood, which serves as a “tumor-normal” control sample, and mutated/malignant tissue and abnormal blood, which serves as a “target” sample. For example, aspects of the present methods also include performing tumor normal analysis from normal cells within a biopsy, e.g., for example where the “target” sample came from. Such methods allow for detecting and diagnosing cell populations from non-mutated tissue or normal blood to determine if mutations are found in familial germlines that may also develop in other places of the body, or if the mutations are somatic to provide for better treatment options.

In some embodiments, the cell/nuclei population within the sample includes one cell population. In some embodiments, the cell/nuclei population within the sample includes two or more, three or more, four or more, five or more, six or more, seven or more, eight or more, nine or more, or ten or more cell populations. In some embodiments, the cell/nuclei population within the sample includes eleven or more, twelve or more, thirteen or more, fourteen or more, fifteen or more, sixteen or more, seventeen or more, eighteen or more, nineteen or more, or twenty or more cell populations.

In some embodiments the cell/nuclei population is in suspension. In some embodiments, the cell suspension comprises a single cell. In some embodiments, the cell suspension comprises a plurality of cells. In some embodiments, the cell population comprises a plurality of cells. In some embodiments, the cell/nuclei population is a single cell. In some embodiments, the cell/nuclei population comprises 1-10 cells. In some embodiments, the cell/nuclei population comprises 3-10 cells. In some embodiments, the cell/nuclei population comprises 3-50 cells. In some embodiments, the cell/nuclei population comprises 2-100 cells. In some embodiments, the cell/nuclei population contains 1 to 1000,000 cells. In some embodiments, the cell/nuclei population contains 1 to 20,000 cells. In some embodiments, the cell/nuclei population contains 1 to 15,000 cells. In some embodiments, the cell/nuclei population contains 1 to 16,000 cells. In some embodiments, the cell/nuclei population contains 1 to 15,000 cells. In some embodiments, the cell/nuclei population contains 1 to 10,000 cells. In some embodiments, the cell/nuclei population contains 1 to 100 cells, 100 to 200 cells, 200 to 300 cells, 300 to 400 cells 400 to 500 cells, 500 to 600 cells, 600 to 700 cells, 700 to 800 cells, 800 to 900 cells, 900 to 1000 cells, 1000 to 1100 cells, 1100 to 1200 cells, 1200 to 1300 cells, 1300 to 1400 cells, or 1400 to 1500 cells. In some embodiments, the cell/nuclei population is contains 20,000 cells or less, 19,000 cells or less, 18,000 cells or less, 17,000 cells or less, 16,000 cells or less, 15,000 cells or less, 14,000 cells or less, 13,000 cells or less, 12,000 cells or less, 11,000 cells or less, 10,000 cells or less, 9,000 cells or less, 8,000 cells or less, 7,000 cells or less, 6,000 cells or less, 5,000 cells or less, 4,000 cells or less, 3,000 cells or less, 2,000 cells or less, 1,500 cells or less, 1,000 cells or less, 500 cells, 250 cells or less, 100 cells or less, 50 cells or less, 25 cells or less, 10 cells or less, 5 cells or less, or 2 cells or less. In some embodiments, the cell/nuclei population contains 1 cell. In some embodiments, the cell/nuclei population contains 1 to 15,000 cells. In some embodiments, the cell/nuclei population contains 1 to 300 cells, 1 to 10 cells, 3 to 10 cells, 10 to 20 cells, 1 to 5 cells, 1 to 15 cells, 1 to 25 cells, 1 to 75 cells, and the like.

In some embodiments, the cell/nuclei population is on a substrate. In some embodiments, the substrate is a slide. In some embodiments, the slide is made from glass.

### Analysis of Sequencing Data

Aspects of the present disclosure also provides a method for analyzing sequencing data, such as those acquired using the library preparation methods described herein. Such methods are implemented by a computer-implemented method, where a user may access a file on a computer system, wherein the file is generated by sequencing multiplexed amplification products from one or more cell populations of a heterogenous sample by, e.g., a method of analyzing a heterogeneous cell population, as described herein. Thus, the file may include a plurality of sequencing reads for a plurality of nucleic acids derived from the heterogenous cell population. Each of the sequencing reads may be a sequencing read of a nucleic acid that contains a target nucleic acid nucleotide sequence (e.g., a nucleotide sequence encoding a target region of interest) and one or more barcode sequences that identifies the cell source (e.g., a cell in a well in a multi-well plate, a capillary, a microfluidic chamber, etc.) from which the nucleic acid originated (e.g., after PCR and/or ligation of the target nucleic acid expressed by the one or more cells in the in the well). In some embodiments, the sequencing read is a paired-end sequencing read.

The sequencing reads in the file may be assembled to generate a consensus sequence of a target nucleic acid nucleotide sequence by matching the nucleotide sequence corresponding to the target nucleic acid nucleotide sequence and the barcode sequences contained in each sequencing read.

Aspects of the present disclosure include analyzing the sequenced indexed libraries.

In some embodiments, analyzing includes identifying, in each of the indexed libraries, whether the indexed libraries contain one or more indexing errors.

In some embodiments, analyzing the sequenced indexed libraries includes correcting one or more indexing errors if an indexing error is present.

In some embodiments, analyzing the sequenced indexed libraries includes removing one or more indexed libraries that does not contain an indexed sequence.

In some embodiments, analyzing the sequenced indexed libraries includes demultiplexing each of the indexed libraries according to each of their barcode sequence.

In some embodiments, demultiplexing includes separating the reads of different indexed libraries, as determined by the barcode sequence, into individual files.

In some embodiments, analyzing the sequenced indexed libraries includes trimming each of the indexed libraries to remove at least a portion of the barcode and/or adapter sequence. In some embodiments, analyzing the sequenced indexed libraries includes trimming each of the indexed libraries to remove the full barcode and/or adapter sequences. The barcode information is kept in the header of the read. Thus, the header information (e.g., barcode) will be carried through to subsequent steps in the bioinformatics analysis.

In some embodiments, analyzing the sequenced indexed libraries includes aligning each of the indexed libraries to a target sequence of the human genome and producing an alignment file for each of the indexed libraries.

In some embodiments, analyzing the sequenced indexed libraries comprises running each of the alignment files through a variant caller configured to identify and quantify genetic alterations within the indexed libraries. A variant caller, used herein in its conventional sense, is an algorithm that calls structural variants and writes them to an output file. In some embodiments, the variant caller includes additional statistical tests in addition to variant identification. In some embodiments, the variant caller does not include additional statistical tests in addition to variant identification.

In some embodiments, the genetic alterations include structural variants. Non-limiting examples of structural variants include, but are not limited to splice variations, somatic mutations, or genetic polymorphisms. In some embodiments, structural variants include genetic variations and mutations associated with cancer. In some embodiments, the structural variants of the one or more populations of cells are compared with cell types with known structural variants using reference samples and variant databases.

In some embodiments, the indexed libraries are aligned to a reference sequence with one or more genome or transcriptome read aligners selected from Burrows Wheeler Aligner (BWA), BWA-MEM, Bowtie2, RNA-STAR, and Salmon. In some embodiments, the reference sequence is a sequence of the human genome. In some embodiments, the reference sequence is a sequence for the target nucleic acid in a reference database, such as GenBank®. Thus, in some embodiments, a target nucleotide sequence in a first sequencing read in a subset of sequencing reads, as described above, is 80% or more, e.g., 85% or more, 90% or more, 95% or more, or up to 100% identical to a reference sequence for the target nucleic acid from a reference database. In some embodiments, the reference sequence is one or more other sequences in sequencing reads of the same subset. Thus, in such cases, a target nucleotide sequence in a first sequencing read in a subset of sequencing reads, as described above, is 80% or more, e.g., 85% or more, 90% or more, 95% or more, or up to 100% identical to a target nucleotide sequence in a second sequencing read in the same subset. In some instances, a target nucleotide sequence in a first sequencing read in a subset is 80% or more, e.g., 85% or more, 90% or more, 95% or more, or up to 100% identical to a target nucleotide sequence in all other sequencing reads in the same subset.

In some embodiments, identifying the genetic alterations within the indexed library includes extracting structural variants from each of the alignment files of the indexed libraries. In some embodiments, extracting structural variants comprises listing all the structural variants commonly found in the alignment file for each indexed library.

In some embodiments, identifying includes identifying at least one of: the percentage of genome reads in a region of the sequence containing a variant, the quality scores of nucleotides in reads covering a variant, and the total number of reads at a variant position. In some embodiments, the quality score is output by the sequencer and tells the user the quality of that nucleotide call by the sequencer. For example, the quality score can be represented by a Phred quality score which is a unique character representing the error rate of that nucleotide call.

In some embodiments, quantifying the structural variants includes determining statistical significance of each structural variant using one of more statistical algorithms to calculate a statistical score and/or a significance value for each of the structural variants.

In some embodiments, the statistical algorithm is a binomial distribution model, over-dispersed binomial model, beta, normal, exponential, or gamma distribution model.

In some embodiments, the structural variants are selected from one of more of: single nucleotide variants (SNVs), small insertions, deletions, indels, and a combination thereof. However, the methods used herein are not limited to such structural variants.

In some embodiments, the genetic variant may be a single nucleotide variant, that is a change from one nucleotide to a different nucleotide in the same position. In some embodiments, the genetic variant may be an insertion or deletion, that adds or removes nucleotides. In some embodiments, the genetic variant may be a combination of multiple events including single nucleotide variants and insertions and/or deletions. In some embodiments, a genetic variant may be composed of multiple genetic variants present in different regions of interest.

Requiring a positive determination for the genetic variant in a plurality of replicate amplification reactions reduces the probability of a false positive determination of the genetic variant being present in a DNA sample. In some embodiments, the method includes requiring multiple positive determinations in replicate amplification reactions.

In some embodiments, the mean frequency and coefficient of variation (CV) at which a given variant is observed (i.e. in sequencing results) as a result of error in the method used to sequence a DNA sample can be used to determine and/or model background levels (i.e. noise) for a genetic variant. These values can be used, for example, to determine cumulative distribution function (CDF) values and/or to calculate z-scores. In turn, measurements and/or models of background noise for a genetic variant can then be used to establish threshold frequencies above which a genetic variant must be observed to be determined as being present in a given amplification reaction (a positive determination). For a positive determination, the frequency of the variant must be higher than the mean frequency at background levels.

In some embodiments, the method includes comparing the frequency of variants to a threshold frequency, wherein the threshold frequency is determined using, for example, a binomial, over-dispersed binomial, Beta, Normal, Exponential or Gamma probability distribution model. In some embodiments, the threshold frequency at which a given genetic variant must be observed at or above to be determined as being present in a replicate amplification reaction is the frequency at which the cumulative distribution function (CDF) value of that genetic variant reaches a predefined threshold value (CDF thresh) of 0.99, 0.995, 0.999, 0.9999, 0.99999 or greater.

In some embodiments of the method of the invention, the threshold frequency is determined using a z-score cut-off. In some embodiments, the background mean frequency and variance of the frequency for the genetic variant determined in step (i) are modelled with a Normal distribution, and the threshold frequency for calling a mutation is the frequency at the z-score which is a number of standard deviations above the background mean frequency. In some embodiments, the threshold frequency is the frequency at z-score of 20. In some embodiments, the threshold frequency is the frequency at z-score of 30.

In some embodiments, establishing a threshold frequency at or above which the genetic variant must be observed in sequencing results of amplification reactions to assign a positive determination for the presence of the genetic variant in a given amplification reaction comprises (a) based on the read count distribution determined for a plurality of genetic variants—which is optionally a normal distribution defined by the mean frequency and variance of the frequency determined for a plurality of genetic variants, establishing a plurality of threshold frequencies at or above which the genetic variants should be observed in sequencing results of amplification reactions to assign a positive determination for the presence of the genetic variant in a given amplification reaction, and (b) based on step (a), establishing an overall threshold frequency at or above which a genetic variant must be observed in sequencing results of a given amplification reaction to assign a positive determination for the presence of the genetic variant in that amplification reaction, which is the threshold frequency at which 90%, 95%, 97.5%, 99% or more of the threshold frequencies determined in step (a) are less than this value. In some embodiments, threshold frequencies need not be determined for each possible base at each position of the region of interest, and an overall threshold based on a plurality of genetic variants can be used in the method of the disclosure.

A computer system for implementing the present computer-implemented method may include any arrangement of components as is commonly used in the art. The computer system may include a memory, a processor, input and output devices, a network interface, storage devices, power sources, and the like. The memory or storage device may be configured to store instructions that enable the processor to implement the present computer-implemented method by processing and executing the instructions stored in the memory or storage device.

The output of the analysis may be provided in any convenient form. In some embodiments, the output is provided on a user interface, a print out, in a database, as a report, etc. and the output may be in the form of a table, graph, raster plot, heat map etc. In some embodiments, the output is further analyzed to determine properties of the cell from which a target nucleotide sequence was derived. Further analysis may include correlating expression of a plurality of target nucleotide sequences within cells, principle component analysis, clustering, statistical analyses, and the like.

### 6.2 Compositions and Kits

Aspects of the present disclosure provides a composition and/or kits for ligation-based or amplicon-based library preparation methods described herein. The composition and/or kit may comprise one or more of the primer sets described herein. The composition and/or kit may comprise one or more of the adapter sequences described herein. The composition and/or kit may also comprise one or more reagents described herein.

Aspects of the present disclosure provides a kit for ligation-based or amplicon-based library preparation in situ.

Aspects of the present disclosure provides a kit for amplicon-based or ligation-based library preparation for a cell/nuclei population for sequencing. The kit may comprise one or more primer sets, oligonucleotides, adapter sequences, reagents, enzymes, and/or buffers described herein used in the amplicon-based or ligation-based methods described in section 6.1.1, 6.1.2, or 6.1.3. The kit may comprise one or more primer sets, oligonucleotides, adapter sequences, reagents, enzymes, and/or buffers described herein at concentrations used in the amplicon-based or ligation-based methods described in section 6.1.1, 6.1.2, or 6.1.3 The kit may further comprise instructions for preparing the ligation-based or amplicon-based methods described herein. The kit may also comprise reagents for performing amplification or ligation techniques (e.g., PCR, amplification, ligation, etc.), enzymatic fragmentation and/or ERA, hybridization capture, barcoding, purification techniques, and/or sequencing (e.g., Next Generation Sequencing). For example, the kit may further comprise enzymes, sample mixtures, lysing agents, purification reagents, amplification reagents (PCR buffers, PCR kits, enzymes, polymerases, and the like), ligation reagents (e.g., reagents for enzymatic fragmentation and/or End-tail A repair, ligases, and the like), and/or sequencing reagents as described previously in section 6.1.1 6.1.2, and 6.1.3 of the methods described herein.

In some embodiments, the kit comprises a pre-processed cell/nuclei population sample, such as a permeabilized and/or fixed cell/nuclei population of the sample, as described in section 6.1.3.

In some embodiments, the kit comprises a specific amount of the cell/nuclei population and reagents as described in sections 6.1.1-6.1.3.

Aspects of the present disclosure include kits for ligation-based library preparation in situ, a kit comprising: a cell preservation agent capable of preserving a cell/nuclei population, the cell preservation agent selected from: a fixative, a permeabilizer, or a fixative and a permeabilizer; a fragmentation enzyme and buffer for performing an enzymatic fragmentation reaction to form DNA or RNA fragments within the cell/nuclei population; an End repair and A tail (ERA) master mix and buffer for performing an end-repair and A-tailing reaction on the one or more DNA or RNA fragments; a ligation enzyme and buffer; adapter sequences, wherein the ligation enzyme and buffer, and adapter sequences are capable of ligating, in each cell, the DNA or RNA fragments to the adapter sequences in situ to create a ligated library comprising ligated DNA or RNA fragments; and a cell lysis buffer; in an amount sufficient to prepare a ligation-based library in situ for sequencing; and instructions for carrying out the ligation-based library preparation in situ, the instructions providing the following steps: performing, in each cell of the cell/nuclei population, an enzymatic fragmentation reaction to form DNA or RNA fragments within the cell/nuclei population; ligating, in each cell, the DNA or RNA fragments to adapter sequences in situ to create the ligated library comprising ligated DNA or RNA fragments; lysing each of the cells to collect the ligated DNA or RNA fragments; purifying the ligated DNA or RNA fragments of the cells; and sequencing the ligated DNA or RNA fragments of the cells.

Adapter sequences in the kit, and instructions in the kit for performing the step of adapter-indexing ligation are described in section 6.1.1.3 “Adapter-indexing ligation”.

In some embodiments, the kit further comprises amplification primers for amplifying the ligated DNA or RNA fragments to form amplicon products. Amplification primers include but are not limited to primers used to hybridize with sample DNA or RNA that define the region to be amplified, sequencing primers, barcoding primers, and the like. In certain embodiments, the kit further comprises a polymerase chain reaction (PCR) enzyme master mix comprising one or more of: an enzyme, a buffer, or an enzyme and a buffer.

In some embodiments, the amplification primers comprise barcoding primers, sequencing primers, or a combination thereof.

In some embodiments, the kit further comprises barcoding primers, and a second PCR Enzyme master mix comprising one or more of: an enzyme, a buffer, or an enzyme and a buffer. In some embodiments, the PCR enzyme master mix of the kit can include a PCR enzyme master mix of a “PCR library mix” or “PCR kit” as described in section 6.1.2.1. In some embodiments, the barcoding primers can be included in an amount sufficient for 8 reactions, 24 reactions, 96 reactions, and the like. In certain embodiments, additional barcoding primers and PCR enzyme master mix can be included in the kit or separately packaged.

In some embodiments, the kit can include a cell lysis buffer as described in section 6.1.1.5. In some embodiments, the kit can further include a protease K or other enzymes used during the lysing step. In some embodiments the additional lysis reagents (including buffer or protease K) can be included in the kit or separately packaged.

The ligation-based kit can include instructions for any additional steps used in the ligation-based method as described in section 6.1.1.

In some embodiments, the kit can further include one or more components selected from: SPRI, PBS, IDTE, antibodies and cell staining reagents, streptavidin magnetic beads, and a combination thereof. For example, the antibodies and cell staining reagents within the kit can be used for cell sorting, and the kit comprises further instructions for carrying out the cell sorting steps as described in section 6.1.1.5 or 6.1.2.3 “cell sorting”. In certain embodiments, antibodies and cell staining reagents for cell sorting can be included in the kit but separately packaged.

In some embodiments where hybrid capture will be performed as described in section 6.1.1.7 “Hybridization Capture”, the kit will include components and instructions necessary for performing hybridization capture. For example, in some embodiments, the kit comprises one or more hybridization capture components selected from: Biotinylated target panel, hybridization buffer, wash buffers, blocking oligonucleotides, PCR Enzyme Master Mix (e.g., enzyme, buffer, or both enzyme and buffer), amplification primers (e.g., P5/P7 amplification primers, barcoding primers, and the like), and a combination thereof. In certain embodiments, the components used for hybridization capture can be included in the kit and separately packaged. When hybridization capture is performed, the kit comprises further instructions for hybridization capture the cell sorting steps as described in section 6.1.1.7 “Hybridization Capture”.

In certain embodiments, the kit can comprise further instructions for carrying out additional steps of the ligation-based method as described in section 6.1.1. The method steps as described in section 6.1.1 can be incorporated as instructional steps included in the kit. For example, when a sorting step is required and the antibody and/or staining reagents for cell sorting is included in the kit, the instructions can further include the step of sorting the cell/nuclei population into subpopulations by phenotypes to determine target cells/nuclei and non-target cells/nuclei. In another example, when a hybridization capture step is required and the hybridization capture components are included in the kit, the instructions can further include the step of performing hybridization capture.

In certain embodiments, the kit can include software for carrying out particular steps as described in the instructions. For example, in some embodiments, the software can include instructions for carrying out analysis of the DNA or RNA within the one or more cell/nuclei populations after sequencing (e.g., sequencing data), as described in section 6.1.3 “Analysis of Sequencing Data”.

Aspects of the present disclosure include a kit for amplicon-based library preparation in situ, comprising a cell preservation agent capable of preserving a cell/nuclei population, the cell preservation agent selected from: a fixative, a permeabilizer, or a fixative and a permeabilizer; a primer pool set capable of amplifying a target sequence region of DNA or RNA within one or more cells of the cell/nuclei population; a polymerase chain reaction (PCR) Enzyme Master Mix comprising one or more of: an enzyme, a buffer, or an enzyme and a buffer; a cell lysis buffer; in an amount sufficient to prepare an amplicon-based library in situ for sequencing; and instructions for carrying out the amplicon-based library preparation in situ, the instructions providing the following steps: amplifying the target sequence region of DNA or RNA in the cell/nuclei population to produce a first set of amplicon products for each cell; lysing each of the cells to isolate DNA or RNA fragments having the target sequence region of DNA or RNA within the first set of amplicon products; purifying the DNA fragments; and sequencing the DNA or RNA fragments of the cells/nuclei population.

In some embodiments, the kit further comprises protease K or other enzymes used during the lysing step. In some embodiments the additional lysis reagents (including buffer or protease K) can be included in the kit or separately packaged.

In some embodiments, the kit further comprises barcoding primers, and a second PCR Enzyme master mix comprising one or more of: an enzyme, a buffer, or an enzyme and a buffer.

In some embodiments, the primer pool set comprises target primer sets as described in section 6.1.1, 6.1.2, or 6.1.3. In particular embodiments, the primer pool set comprise target primer sets as described in section 6.1.2.1.

In some embodiments, the PCR enzyme master mix of the kit can include a PCR enzyme master mix of a “PCR reaction mixture” “PCR library mix” or “PCR kit” as described in section 6.1.2.1.

In some embodiments, the kit can include a cell lysis buffer as described in section 6.1.1.5. In some embodiments, the kit can further include a protease K used during the lysing step.

In some embodiments, the kit further comprises barcoding primers, and a second PCR Enzyme master mix comprising one or more of: an enzyme, a buffer, or an enzyme and a buffer. The PCR enzyme master mix of the kit can include a PCR enzyme master mix of a “PCR reaction mixture” “PCR library mix” or “PCR kit” as described in section 6.1.2.1. In some embodiments, an amount of barcoding primers can include an amount sufficient for a 8 sample reaction, a 24 sample reaction, a 96 sample reaction, and the like. In certain embodiments, additional barcoding primers and PCR enzyme master mix can be included in the kit and separately packaged.

In some embodiments, the kit can further include one or more of: SPRI, PBS, IDTE, antibodies and cell staining reagents, or a combination thereof. In some embodiments, the antibodies and cell staining reagents within the kit can be used for cell sorting, and the kit comprises further instructions for carrying out the cell sorting steps as described in section 6.1.1.5 or 6.1.2.3 “cell sorting”. In certain embodiments, antibodies and cell staining reagents for cell sorting can be included in the kit and separately packaged.

In some embodiments, the kit further comprises adapter sequences, e.g., for carrying out a second amplification step. For example, if a second amplification step is required, the kit can include instructions for carrying out the step of amplifying the first set of amplicon products with adapter sequences to produce a second set of amplicon products.

In certain embodiments, the kit can comprise further instructions for carrying out additional steps of the amplicon-based method as described in section 6.1.2. The method steps as described in section 6.1.2 can be incorporated as instructional steps included in the kit. For example, when a sorting step is required and the antibody and/or staining reagents for cell sorting is included in the kit, the instructions can further include the step of sorting the cell/nuclei population into subpopulations by phenotypes to determine target cells/nuclei and non-target cells/nuclei.

In certain embodiments, the kit can include software for carrying out particular steps as described in the instructions. For example, in some embodiments, the software can include instructions for carrying out analysis of the DNA or RNA within the one or more cell/nuclei populations after sequencing (e.g., sequencing data), as described in section 6.1.3 “Analysis of Sequencing Data”.

The kits (e.g., ligation-based kit or amplicon-based kit) of the present disclosure can include instructions in various forms, e.g., written form, digital form, CD-ROM, DVD, flash drive, hard drive, etc.) Additionally, a computer system for implementing the present method, kit instructions, and a software may include any arrangement of components as is commonly used in the art. The computer system may include a memory, a processor, input and output devices, a network interface, storage devices, power sources, and the like. The memory or storage device may be configured to store instructions that enable the processor to implement the present computer-implemented method or software by processing and executing the instructions stored in the memory or storage device.

The components of the kits (e.g. ligation-based kit and amplicon-based kit) of the present disclosure can be packaged separately, in multiple containers or packages, or in a single containers or packages. For example, a ligation-based kit comprising components with similar storage temperatures can be packaged together in 1 container or package, while the remaining components of the kit can be packaged in a separate container or package. Thus, each component within each kit can be packaged together, with one or more other components of the kits, or separately, depending on the storage conditions and needs of the user.

### 7. EXAMPLES

The following examples are put forth so as to provide those of ordinary skill in the art with a complete disclosure and description of how to make and use the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### 7.1 Example 1: In Situ Amplicon-Based Library Preparation

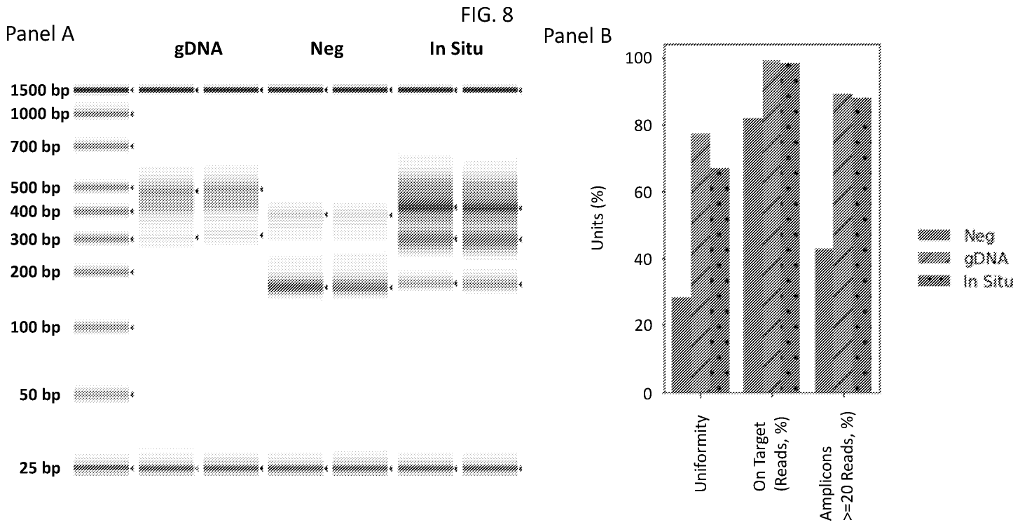

Amplicon based Library preparation was performed on genomic DNA (gDNA) according to established manufacturer protocols. An in situ protocol was developed and performed on in situ cells (in situ) and a negative control consisting of PBS (Neg) (see experiment protocols, example 1). In brief, cells were fixed and permeabilized. PCR1 reagents were then added to a cell suspension of these fixed and permeabilized cells and PCR performed. Cells were then pelleted, and cell supernatant removed ensuring the only the cells and products amplified in the cells are carried through to the next steps, which include cell lysis and PCR2 amplification on the purified cell lysate. As shown in FIG. 8, amplified libraries were run on a TapeStation HSD1000 (Agilent), showing product sized after the two PCR steps (Panel A). Libraries were not identical to gDNA, however appear to have amplification product in similar size ranges indicating amplification of the targets was occurring (fragments between 300 bp and 600 bp). The product around 180 bp was likely primer dimer. Sequencing libraries confirmed amplification of target amplicons (Panel B).

### Materials and Methods:

Step 1: Cell Fixation

2.5×105 cells were fixed and permeabilized using IncellMax (IncellDx), a fixation/permeabilizer, according to the following protocol: The suspension containing desired number of cells was diluted with 1 ml PBS and pelleted at 300×g for 5 minutes. The supernatant was removed, and the cell pellet was resuspended in 250 μl of 1×IncellMax reagent, followed by a 1-hour incubation at room temp. Cells were then washed with PBS by centrifuging at 1,500×g for 5 minutes, removing the supernatant and then resuspended with PBS.

Step 2: In Situ Library Preparation—Amplicon

16,000 fixed cells were amplified in situ using rhAmpSeq Library Kit (IDT) using a mix of the Sample ID panel and a custom target panel by performing the PCR1 step according to protocol recommendations, except for the enzyme deactivation step, which was eliminated. In brief, the 16,000 cells were pelleted and resuspended in 11 μl of PBS and then the following was added to the cell suspension: 5 μl 4× rhAmpSeq Library Mix 1 and 41 each 10×library primer pool. Cells then underwent PCR amplification, by activating the enzyme for 10 min at 95*C and then amplifying for 14 cycles with 15 sec at 95*C then 8 min at 61*C. Following PCR, cells were immediately centrifuged at 1,500×g for 5 minutes, the supernatant removed, and cells resuspended with 25 μl PBS.

Step 3: Cell Lysis and Amplicon Purification

Resuspended cells were lysed by adding 25 μl Buffer AL (Qiagen) and 5 μl Protease or proteinaseK (Qiagen) and incubating at 70*C for 10 minutes. PCR amplicons were then purified using 0.8×SPRIselect beads (BeckmanCoulter), washed with 80% ethanol and eluted in 20 μl IDTE (IDT).

Step 4: Library Amplification

Purified rhAmpSeq PCR1 amplicons were diluted 1:20 and then 11 μl is amplified using 5 μl of 4×rhAmpSeq Library Mix 2, and 2 μl each of 1 uM Indexing PCR Primers. Amplicons were further amplified by activating the enzyme for 3 min at 95*C, amplifying for 24 cycles with 95*C for 15 sec, 60*C for 30 sec, and 72*C for 30 sec, followed by a final extension at 72*C for 1 min. Amplicons were purified using 1×SPRIselect beads (BeckmanCoulter), washed with 80% ethanal and eluted in 20 μl IDTE (IDT).

Step 5: Sequencing

Amplified libraries were quantified using HighSensitivity D1000 Tapestation (Agilent) and KAPA Library Quant Kit (Roche) and then sequencing on an Illumina MiSeq (Illumina) according to the manufacturer recommendations. Reads were mapped to the human genome (hg38) using BWA MEM. Performance metrics including coverage and uniformity were determined using a combination of picard tools and custom algorithms.

Results:

Libraries were generated from samples in which in situ PCR was performed. Amplification products occurred in the expected size range (fragments between 300 bp and 600 bp), with formation of a primer dimer (fragments at 180 bp). Sequencing libraries confirmed amplification of target amplicons (Panel B).

### 7.2 Example 2: In Situ Amplicon-Based Library Preparation, V2

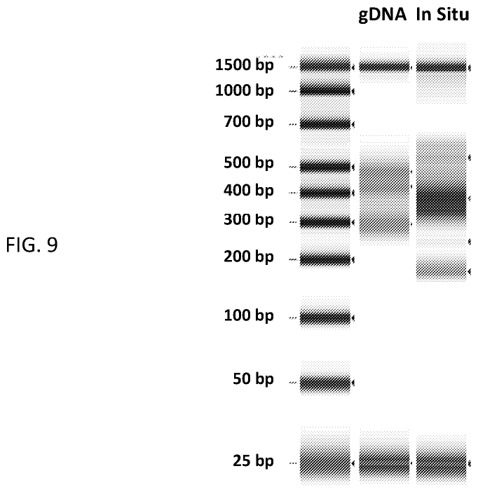

Amplicon Library preparation was performed on genomic DNA (gDNA) according to established manufacturer protocols (IDT). An in situ protocol using multiple in situ PCR steps was performed on cells (in situ) (see materials and methods). In brief, cells were fixed and permeabilized, one set of PCR reagents were added to a cell suspension of these fixed and permeabilized cells and PCR performed. Cells were then pelleted, and cell supernatant removed ensuring the only the cells and products amplified and currently in the cells are carried through to the next steps, which include adding PCR2 reagents to the cells and performing PCR2 in situ. Cells pelleted once again, and the cells lysed, and PCR products cleaned up. Amplified libraries were run on a TapeStation HSD1000 (Agilent), showing product sized after the two PCR steps as shown in FIG. 9.

### Materials and Methods:

Step 1: Cell Fixation

2.5×105 cells were fixed and permeabilized using IncellMax fixation/permeabilizer (IncellDx) according to manufacturer recommendations for 1 hour. Cells were then washed with PBS by centrifuging at 1,500×g for 5 minutes, removing the supernatant and then resuspended with PBS.

Step 2: In Situ Library Preparation—Amplicon

16,000 fixed cells were amplified in situ using rhAmpSeq Library Kit (IDT) using a mix of the Sample ID panel and a custom target panel by performing the PCR1 step according to protocol recommendations, except for the enzyme deactivation step, which was eliminated. In brief, the 16,000 cells were pelleted and resuspended in 11 μl of PBS and then the following was added to the cell suspension: 5 μl 4× rhAmpSeq Library Mix 1 and 41 each 10×library primer pool. Cells then underwent PCR amplification, by activating the enzyme for 10 min at 95*C and then amplifying for 14 cycles with 15 sec at 95*C then 8 min at 61*C. Following PCR, cells were incubated at 80*C. Then, the cells were centrifuged at 1,500×g for 5 minutes, the supernatant removed, and cells resuspended with 11 μl PBS.

Step 3: Library Amplification

rhAmpSeq PCR2 was then performed in situ by adding the following to the cell suspension: 5 μl 4×rhAmpSeq Library Mix 2, 2 μl each 1 uM indexing primer. Following PCR2, cells were immediately centrifuged at 1,500×g for 5 minutes, the supernatant removed, and cells resuspended with 25 μl PBS.

Step 4: Cell Lysis and Amplicon Purification

Resuspended cells were lysed by adding 25 μl Buffer AL (Qiagen) and 5 μl Protease or proteinaseK (Qiagen) and incubating at 70*C for 10 minutes. PCR amplicons were then purified using 1×SPRIselect beads (BeckmanCoulter) and washed with 80% ethanol and eluted in 20 μl IDTE (IDT).

Results

PCR products were observed after the two in situ PCR steps.

### 7.3 Example 3: In Situ Amplicon-Based Library Preparation with Cell Sorting—Scatter

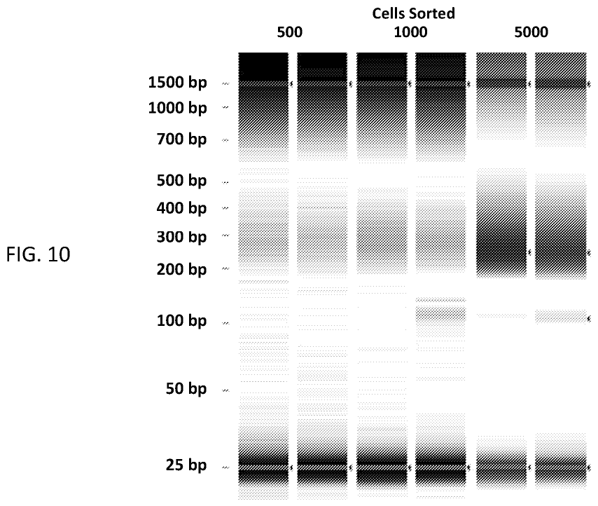

In situ amplicon library preparation was performed on 16K and 32K fixed and permeabilized cells. After PCR1, the cells were pelleted and resuspended in PBS, followed by sorting individual cells based on forward scatter and backscatter properties on a SONY SH800S, no dyes, stains or fluorophores were added to the cells. Subpopulations of 500, 1000, or 5000 cells were isolated, lysed and amplified using indexed primers. Then ran on a TapeStation HSD1000 (Agilent), indicating amplification product in all subpopulations as shown in FIG. 10.

### Materials and Methods

Performed the protocol as described in Example 1 with the following modifications:

### Example 1 Step 2

Either 16,000 fixed cells or 32,000 fixed cells were used as input to the PCR reaction. Cells were resuspended in 100 μl of PBS after the post-PCR centrifugation.

### Example 1 Step 3

Buffer AL and Protease K were not added to the reaction since they were already added to the sorted tubes.

### Example 1 Step 4

Sequencing was not performed on these samples.

The following steps were added between Example 1 Step 2 and Example 1 Step 3:

Step 2a:

1.5 μl microfuge tubes were prepared for cell sorting by adding 25 μl of Lysis buffer and 5 μl of protease/proteinase K to each tube. Control samples were used to set up the cell sorting instrument and gates. Once set up was complete, the reactions were loaded on the cell sorter, a SONY SH800S. Events were first classified as single intact cells by the forward scatter and backscatter and then sorted into the wells until each well had the appropriate number of cells added to it. After sorting, volumes of each tube were measured and adjusted to 55 μl using PBS.

### Results

Cells (defined by forward scatter and back scatter properties of the instruments) were identifiable with flow cytometry. After isolating different size populations of cells, libraries were able to be generated from all of the populations. Due to the large amount of dilution that occurs to the buffer the cells are in, these results indicate that the amplicons are inside of the cells and not carry over from the supernatant.

### 7.4 Example 4: In Situ Amplicon-Based Library Preparation with Cell Sorting—CD45

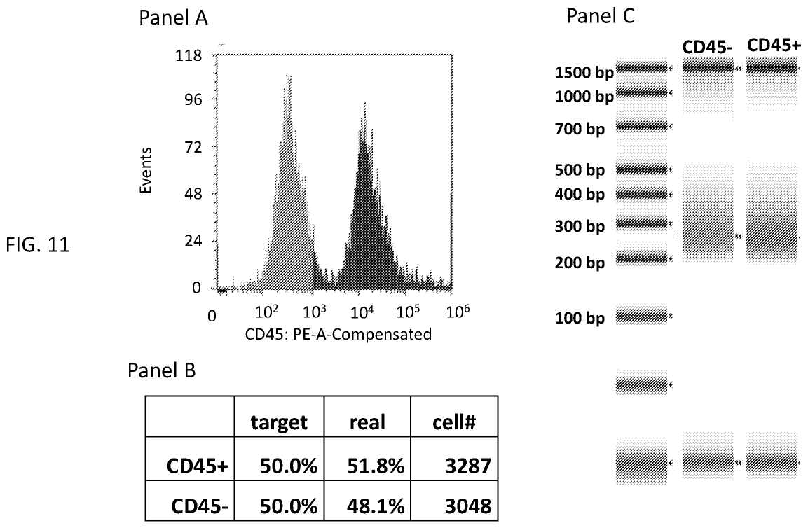

In situ amplicon library preparation was performed on two populations of fixed and permeabilized cells. After PCR1, the cells were pelleted and resuspend in cell staining buffer (Biolegend) and then stained according to the experiment protocol below for either CD45-PE or IgG-PE. Cells were mixed and then sorted on a SONY SH800S based on PE fluorescence intensity as shown in FIG. 11. FIG. 11, panel (A) contains a histogram of the fluorescence intensities, FIG. 11, panel (B) contains cell numbers and percentages total observed, and FIG. 11, panel (C) shows size profile of the library after PCR2 amplification with TapeStation HSD1000 (Agilent).

### Materials and Methods

Performed the protocol as described in Example 1 with the following modifications:

### Example 1 Step 2

After PCR1, the cells were not pelleted.

### Example 1 Step 3

Buffer AL and Protease K were not added to the reaction since they were already added to the sorted tubes.

### Example 1 Step 4

Sequencing was not performed on these samples.

The following steps were added between Example 1 Step 2 and Example 1 Step 3:

Step 2a

PCR amplified cells were resuspended with 180 μl of Cell Staining Buffer (Biolegend), then centrifuged, supernatant removed and resuspended in 100 μl of Cell Staining Buffer. 5 μl of Human TruStain Fcx (Biolegend) was added and incubated for 5 minutes. Then 1 μl CD45-PE (200 ng/μ1, H130, Biolegend) was added to one reaction and 1 μl IgG1-PE (200 ng/μl, MOPC-21, Biolegend) was added to another reaction, before incubating at 4*C for 1 hour in the dark.

The reactions were washed by increasing the reaction volume to 200 μl with Cell Staining buffer, centrifuging at 1,500×g for 5 minutes, removing supernatant and repeating 2 more times. Cells were then resuspended in 100 μl of Cell Staining buffer and sorted ASAP.

Step 2b

1.5 μl microfuge tubes were prepared for cell sorting by adding 25 μl of Lysis buffer and 5 μl of protease/proteinase K to each tube. Control samples were used to set up the cell sorting instrument and gates, however, once set up was complete, half of the CD45-PE stained reaction was mixed with half of the IgG-PE stained reaction and loaded on the cell sorter, a SONY SH800S. Events were first classified as single intact cells by the forward scatter and backscatter. Single intact cells containing PE signals above 103 were classified as CD45+, while ones below were classified as CD45− and sorted to appropriate tubes.

After sorting, volumes of each tube were measured and adjusted to 55 μl using PBS.

Results

PE signal was observed in approximately 50% of the observed cells, indicating the CD45-PE stained cells bound more antibody than the IgG-PE antibody (which indicates non-specific binding occurring on the cells). PCR amplicons were observed after the cell populations were lysed and amplified with PCR2, indicating the amplicons are definitively inside of the cells, due to the washes and dilution occurring during the staining procedure.

### 7.5 Example 5: In Situ Ligation Based Library Preparation

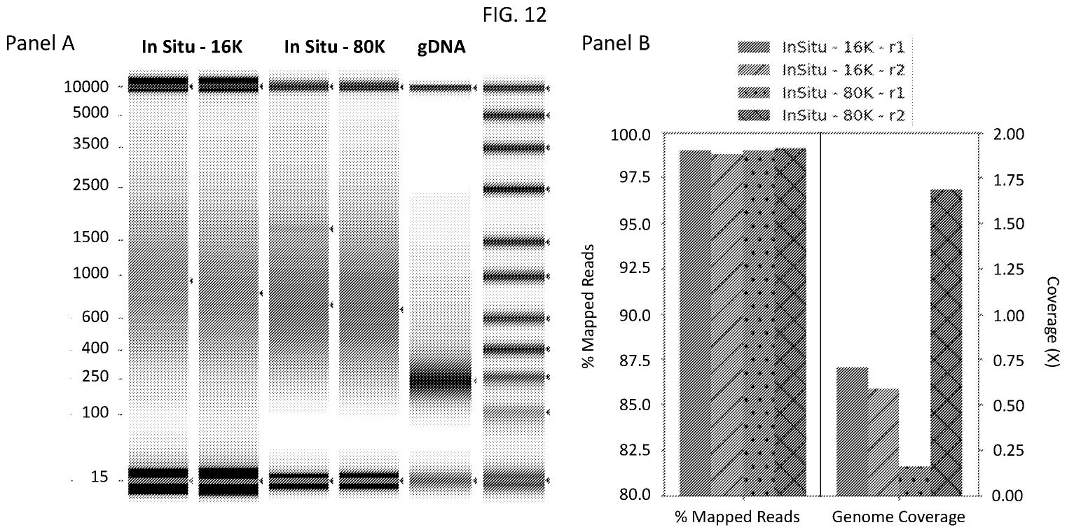

A Library preparation according to the methods of the present disclosure was performed on genomic DNA (gDNA) according to established manufacturer protocols. An in situ protocol was developed and performed on in situ cells (in situ) (see experiment protocols). In brief, cells were fixed and permeabilized using IncellMax (IncellDx). Fixed and permeabilized cells were heat denatured followed by enzymatic fragmentation, End Repair and A-tailing using our reagents. After incubation at recommended temperatures, and inactivation of the enzymes, cells were pelleted and resuspended in a Ligation Master mix. Post ligation, and ligase inactivation, the cells were pelleted and resuspending in a PCR amplification reaction, followed by another round of cell pelleting. After which the cells were lysed and amplified libraries were purified and then run on a TapeStation HSD5000 (Agilent) as shown in FIG. 12, showing product sized after the library preparation as shown in FIG. 12, panel (A). Libraries are not identical to gDNA, due to differences in efficiency of enzymatic fragmentation, however amplification products are present in the samples, as indicated by the gel. Samples were then sequenced to confirm these products contain the required sequences for Illumina sequencing. And 99% of reads sequenced are mapping to the human genome FIG. 12, panel (B). Genome coverage is low, however, that is due to sequencing depth, which was low.

### Materials and Methods

Step 1: Cell Fixation

2.5×105 cells were fixed and permeabilized using IncellMax (IncellDx), a fixation/permeabilizer, according to the following protocol: The suspension containing desired number of cells was diluted with 1 ml PBS and pelleted at 300×g for 5 minutes. The supernatant was removed, and the cell pellet was resuspended in 250 μl of 1×IncellMax reagent, followed by a 1-hour incubation at room temp. Cells were then washed with PBS by centrifuging at 1,500×g for 5 minutes, removing the supernatant and then resuspended with PBS.

Step 2: In Situ Library Preparation—Ligation

NGS libraries were prepared from 16,000 or 80,000 fixed cells using a modified Library Preparation and Amplification Protocol from the Library Preparation and Amplification Kit. In brief, the 16,000 or 80,000 fixed cells were pelleted and resuspended in 34 μl PBS. Cells were incubated at 95*C for 20 min, then 4 μl of Frag/AT Buffer and 12 μl Frag/AT Enzymes were added to the cells, followed by incubation at 37*C for 60 minutes and incubation at 65*C for 30 min. After incubation, the 5 μl of XGen Stubby Adapters (IDT) and 20 μl of the Ligation Master Mix were added to the cells with gentle mixing via pipetting. The ligation reaction was incubated at 20*C for 15 min, followed by ligase inactivation at 65*C for 10 min. Samples were centrifuged at 1,500×g for 5 min, supernatant removed, and resuspended in 20 μl.

Step 3: Library Amplification

In Situ ligated libraries were then amplified by adding 25 μl 2× Library Amplification Hot Start Master Mix and 5 μl of the xGen UDI primer Mix (IDT) performing the following PCR program: Denaturation using 98*C for 45 sec, 5 cycles of 95*C for 15 sec, 60*C for 30 sec and 72*C for 30 sec, and a final extension at 72*C for 60 sec. Cells were then pelleted using 1,500×g, supernatant removed, and cells resuspended in 25 μl PBS.

Step 4: Cell Lysis and Amplicon Purification

Resuspended cells were lysed by adding 25 μl Buffer AL (Qiagen) and 5 μl Protease or proteinaseK (Qiagen) and incubating at 70*C for 10 minutes. Libraries were then purified using 1× SPRIselect beads (BeckmanCoulter) and washed with 80% ethanol and eluted in 20 μl IDTE (IDT).

Step 5: Sequencing

Amplified libraries were quantified using HighSensitivity D1000 Tapestation (Agilent) and KAPA Library Quant Kit (Roche) and then sequencing on an Illumina MiSeq (Illumina) according to the manufacturer recommendations. Reads were mapped to the human genome (hg38) using BWA MEM. Performance metrics including coverage and uniformity were determined using a combination of picard tools and custom algorithms.

Results

A peak of enzymatically fragmented, ligated, and amplified libraries were observed in the in situ samples, with more library observed in the samples with more cells. Fragment size is much larger than that of gDNA, however, libraries were still pooled and sequenced to verify that the peaks contained sequence-able library. Sequencing showed a high percentage of mapped reads associated with each of the sample indexes used, showing these libraries contained sequence-able material.

### 7.6 Example 6: In Situ Ligation-Based Library Preparation with Cell Sorting—CD45

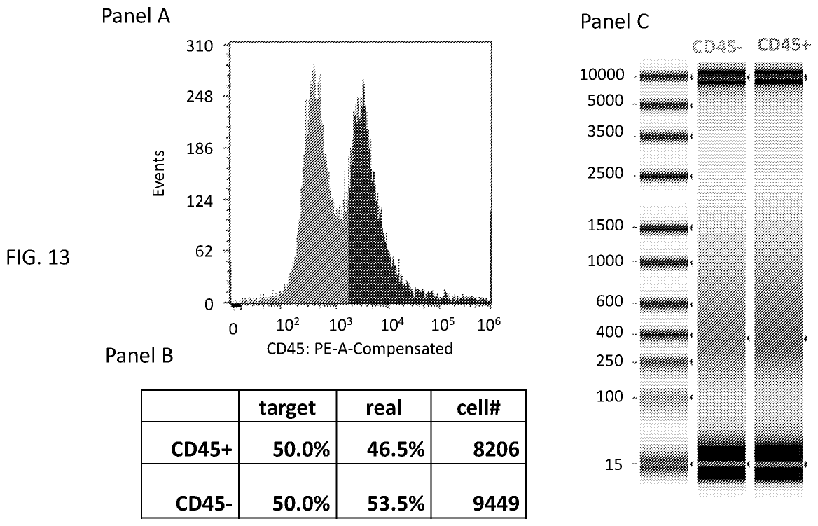

In situ ligation library preparation was performed on two populations of fixed and permeabilized cells. After the PCR step, the cells were pelleted and resuspend in cell staining buffer (Biolegend) and then stained according to the experiment protocol below for either CD45-PE or IgG-PE. Cells were mixed and then sorted on a SONY SH800S based on PE fluorescence intensity as shown in FIG. 13. FIG. 13, panel (A) contains a histogram of the fluorescence intensities, FIG. 13, panel (B) contains cell numbers and percentages total observed, and FIG. 13, panel (C) shows size profile of the library after PCR2 amplification with TapeStation HSD5000 (Agilent).

Performed the protocol as described in Example 5 with the following modifications:

### Example 5 Step 3

After PCR, the cells were not pelleted.

### Example 5 Step 4

Buffer AL and Protease K were not added to the reaction since they were already added to the sorted tubes.

### Example 5 Step 5

Sequencing was not performed on these samples.

The following steps were added between Example 5 Step 3 and Example 5 Step 4:

Step 3a

PCR amplified cells were resuspended with 180 μl of Cell Staining Buffer (Biolegend), then centrifuged, supernatant removed and resuspended in 100 μl of Cell Staining Buffer. 5 μl of Human TruStain Fcx (Biolegend) was added and incubated for 5 minutes. Then 1 μl CD45-PE (200 ng/μl, H130, Biolegend) was added to one reaction and 1 μl IgG1-PE (200 ng/μl, MOPC-21, Biolegend) was added to another reaction, before incubating at 4*C for 1 hour in the dark.

The reactions were washed by increasing the reaction volume to 200 μl with Cell Staining buffer, centrifuging at 1,500×g for 5 minutes, removing supernatant and repeating 2 more times. Cells were then resuspended in 100 μl of Cell Staining buffer and sorted ASAP.

Step 3b

1.5 μl microfuge tubes were prepared for cell sorting by adding 25 μl of Lysis buffer and 5 μl of protease/proteinase K to each tube. Control samples were used to set up the cell sorting instrument and gates, however, once set up was complete, half of the CD45-PE stained reaction was mixed with half of the IgG-PE stained reaction and loaded on the cell sorter, a SONY SH800S. Events were first classified as single intact cells by the forward scatter and backscatter. Single intact cells containing PE signals above 103 were classified as CD45+, while ones below were classified as CD45− and sorted to appropriate tubes.

After sorting, volumes of each tube were measured and adjusted to 55 μl using PBS.

Results

PE signal was observed in approximately 50% of the observed cells, indicating the CD45-PE stained cells bound more antibody than the IgG-PE antibody (which indicates non-specific binding occurring on the cells). Amplification product was observed after the cell populations were lysed and amplified with PCR2, indicating the amplified ligated fragments are definitively inside of the cells, due to the washes and dilution occurring during the staining and sorting procedure.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for preparing a ligation-based library in situ for sequencing, the method comprising:
(a) providing a sample comprising a heterogenous cell/nuclei population having a plurality of phenotypes;
(b) performing, in each cell/nuclei of the heterogenous cell/nuclei population, an enzymatic fragmentation reaction to form DNA fragments within the heterogenous cell/nuclei population;
(c) ligating, in each cell/nuclei, the DNA fragments to adapter sequences in situ to create a ligated library comprising ligated DNA fragments;
(d) sorting the cell/nuclei of the heterogenous cell/nuclei populations into subpopulations by phenotypes to determine target cells/nuclei and non-target cells/nuclei;
(e) lysing each of the target cells/nuclei to collect the ligated DNA fragments;
(f) purifying the ligated DNA fragments; and
(g) sequencing the ligated DNA fragments.

2. The method of claim 1, wherein after step (c), but before step (e), the method further comprises amplifying the ligated DNA fragments to form amplicon products.

3. The method of any one of claims 1-2, wherein after step (e), but before step (g), the method further comprises amplifying the ligated DNA fragments to form amplicon products.

4. The method of any one of claims 1-3, wherein after step (e) but before step (g) the method comprises ligating the ligated DNA fragments with barcode adapter sequences.

5. The method of any one of claims 1-4, wherein the method comprises, before step (a), adding primary antibodies to the sample, and wherein the method comprises, before step (d), adding detectable secondary antibodies or other detectable molecules to the sample.

6. The method of any one of claims 1-5, wherein the method comprises, before step (d), adding primary antibodies, followed by a detectable secondary antibody or other detectable molecule to the sample

7. The method of any one of claims 1-6, wherein the method comprises, before step (d), adding a detectable primary antibody to the sample.

8. The method of any one of claims 1-7, wherein before step (c), performing an end-repair and A-tailing reaction on the one or more DNA fragments.

9. The method of any one of claims 1-8, wherein the end-repair and A-tailing reaction and the enzymatic fragmentation reaction is a single reaction.

10. The method of any one of claims 1-9, where multiple PCR reactions are performed between steps (c) and (g).

11. The method of any one of claims 1-10, wherein ligating the DNA fragments to the adapter sequences comprises running the DNA fragments and adapter sequences in a thermocycler at a temperature and duration sufficient to ligate the DNA fragmented to the adapter sequences.

12. The method of any one of claims 1-11, wherein the adapter sequences comprise Y-adapter nucleotide sequences, hairpin nucleotide sequences, or duplex nucleotide sequences.

13. The method of any one of claims 1-12, wherein said contacting in step (e) comprises amplifying the ligated library to produce a barcoded indexed library.

14. The method of any one of claims 1-13, wherein the barcode adapter sequences comprise a set of forward and/or reverse barcoding adapters.

15. The method of any one of claims 1-14, wherein ligating the ligated DNA fragments with forward and/or reverse barcode adapters produce a barcoded indexed library.

16. The method of any one of claims 1-15, wherein the method further comprises, before step (h), performing hybridization capture on the ligated DNA fragments.

17. The method of any one of claims 1-16, wherein the method further comprises, before step (h), performing hybridization capture on the barcoded indexed library.

18. The method of any one of claims 1-17, wherein said ligating the barcode adapter sequences occurs before sorting in step (d), after step (d) but before step (e), or after step (e).

19. The method of any one of claims 1-18, wherein before step (b), the method comprises fixing and/or permeabilizing the heterogenous cell population.

20. The method of any one of claims 1-19, wherein said sequencing comprises next generation sequencing.

21. The method of any one of claims 1-20, wherein each population of target cells comprises 3-10 cells.

22. The method of any one of claims 1-21, wherein the sample is a cell suspension generated from a tissue sample or a cell suspension generated from a liquid biopsy.

23. The method of any one of claims 1-22, wherein the sample is a Formalin-Fixed Paraffin-Embedded (FFPE) tissue sample or a cryopreserved tissue sample.

24. A method for preparing an amplicon-based library in situ for sequencing, the method comprising:
(a) providing a sample comprising a heterogenous cell/nuclei population having a plurality of cell/nuclei phenotypes;
(b) amplifying, in each cell/nuclei within the heterogenous population, DNA with a primer pool set to produce a first set of amplicon products for each cell/nuclei;
(c) sorting the cell/nuclei phenotypes of the heterogenous cell populations into subpopulations by phenotypes to determine target cells and non-target cells;
(d) lysing each of the target cells to isolate DNA fragments from the first set of amplicon products;
(e) purifying the first set of amplicon products of the target cells; and
(f) sequencing the first set of amplicon products of the target cells.

25. The method of claim 24, wherein before step (b), the method comprises fixing and/or permeabilizing the heterogenous cell population.

26. The method of any one of claims 24-25, wherein before step (d), wherein the method further comprises amplifying the first set of amplicon products with adapter sequences to produce a second set of amplicon products.

27. The method of any one of claims 24-26, wherein the method further comprises, after step (c) or (d), contacting the first set of amplicon products with barcoding sequences.

28. The method of any one of claims 24-27, wherein said barcoding sequences comprise a set of forward and/or reverse barcoding primers, and wherein the method comprises amplifying the first set of amplicon products with the set of forward and/or reverse barcoding primers to produce a barcoded indexed library comprising barcoded amplicon products.

29. The method of any one of claims 24-28, wherein said barcoding sequences comprise a set of forward and/or reverse barcoding adapters, and wherein the method comprises ligating the set of forward and/or reverse barcode adapters to produce a barcoded indexed library comprising barcoded amplicon products.

30. The method of any one of claims 24-29, wherein before step (b), the method comprises fixing and/or permeabilizing the heterogenous cell population.

31. The method of any one of claims 24-30, wherein the primer pool set comprises primers that hybridize to a target region of a target sequence of the DNA within the heterogenous cell population.

32. The method of any one of claims 24-31, wherein the primer pool set further comprises indexing primers.

33. The method of any one of claims 24-32, wherein the sample is a cell suspension generated from a tissue sample or a cell suspension generated from a liquid biopsy.

34. The method of any one of claims 24-33, wherein the sample is a Formalin-Fixed Paraffin-Embedded (FFPE) tissue sample or a cryopreserved tissue sample.

35. The method of any one of claims 24-34, wherein said sequencing comprises next generation sequencing.

36. The method of any one of claims 24-35, wherein said contacting occurs before or after sorting in step (c).

37. The method of any one of claims 24-36, wherein said contacting occurs after lysing in step (d).

38. The method of any one of claims 24-37, wherein each population of target cells comprises 3-10 cells.

39. The method of any one of claims 24-38, where multiple PCR reactions are performed between steps (c) and (f).

40. A method for preparing a ligation-based library in situ for sequencing, the method comprising:
(a) providing a sample comprising a cell/nucleic population;
(b) performing, in each cell of the cell/nuclei population, an enzymatic fragmentation reaction to form DNA fragments within the cell/nuclei population;
(c) ligating, in each cell, the DNA fragments to adapter sequences in situ to create a ligated library comprising ligated DNA fragments;
(d) lysing each of the cells to collect the ligated DNA fragments;
(e) purifying the ligated DNA fragments; and
(f) sequencing the ligated DNA fragments.

41. The method of claim 41, wherein the method comprises, after step (c), sorting the cell/nuclei population into subpopulations:
by phenotypes to determine target cells/nuclei and non-target cells/nuclei; or
irrespective of phenotype.

42. The method of any one of claims 40-41, wherein the method comprises, after step (c), but before step (d), the method further comprises amplifying the ligated DNA fragments to form amplicon products.

43. The method of any one of claims 40-42, wherein after step (d), but before step (0, the method further comprises amplifying the ligated DNA fragments with amplification primers to form amplicon products.

44. The method of any one of claims 40-43, wherein after step (d) but before step (f) the method comprises ligating the ligated DNA fragments with barcode adapter sequences.

45. The method of any one of claims 40-44, wherein the method comprises, before step (a), adding primary antibodies to the sample, and wherein the method comprises, before step (d), adding detectable secondary antibodies or other detectable molecules to the sample.

46. The method of any one of claims 40-45, wherein the method comprises, before step (d), adding primary antibodies, followed by a detectable secondary antibody or other detectable molecule to the sample

47. The method of any one of claims 40-46, wherein the method comprises, before step (d), adding a detectable primary antibody to the sample.

48. The method of any one of claims 40-47, wherein before step (c), performing an end-repair and A-tailing reaction on the one or more DNA fragments.

49. The method of any one of claims 40-48, wherein the end-repair and A-tailing reaction and the enzymatic fragmentation reaction is a single reaction.

50. The method of any one of claims 40-49, where multiple PCR reactions are performed between steps (c) and (f).

51. The method of any one of claims 40-50, wherein ligating the DNA fragments to the adapter sequences comprises running the DNA fragments and adapter sequences in a thermocycler at a temperature and duration sufficient to ligate the DNA fragmented to the adapter sequences.

52. The method of any one of claims 40-51, wherein the adapter sequences comprise Y-adapter nucleotide sequences, hairpin nucleotide sequences, or duplex nucleotide sequences.

53. The method of any one of claims 40-52, wherein the method comprises, after step (d), contacting the ligated DNA fragments with a set of forward and/or reverse barcoding primers, and amplifying the ligated DNA fragments to produce a barcoded indexed library.

54. The method of any one of claims 40-53, wherein the barcode adapter sequences comprise a set of forward and/or reverse barcoding adapter sequences.

55. The method of any one of claims 40-54, wherein ligating the ligated DNA fragments with forward and/or reverse barcode adapter sequences produce a barcoded indexed library.

56. The method of any one of claims 40-55, wherein the method further comprises, before step (f), performing hybridization capture on the ligated DNA fragments.

57. The method of any one of claims 40-56, wherein the method further comprises, before step (f), performing hybridization capture on the barcoded indexed library.

58. The method of any one of claims 40-57, wherein said ligating the forward and/or reverse barcode adapter sequences occurs before sorting, after sorting but before purifying in step (e), or after purifying in step (e).

59. The method of any one of claims 40-58, wherein before step (b), the method comprises fixing and/or permeabilizing the heterogenous cell population.

60. The method of any one of claims 40-59, wherein said sequencing comprises next generation sequencing.

61. The method of any one of claims 40-60, wherein the cell population comprises target cells having 3-10 cells.

62. The method of any one of claims 40-61, wherein the sample is a cell suspension generated from a tissue sample or a cell suspension generated from a liquid biopsy.

63. The method of any one of claims 40-62, wherein the sample is a Formalin-Fixed Paraffin-Embedded (FFPE) tissue sample or a cryopreserved tissue sample.

64. A method for preparing an amplicon-based library in situ for sequencing, the method comprising:
(a) providing a sample comprising a cell/nuclei population;
(b) amplifying, in each cell within the cell/nuclei population, DNA with a primer pool set to produce a first set of amplicon products for each cell;
(c) lysing each of the cells to isolate DNA fragments within the first set of amplicon products;
(d) purifying the DNA fragments; and
(e) sequencing the DNA fragments.

65. The method of claim 64, wherein the method comprises, after step (b), sorting the cell/nuclei population into subpopulations by phenotypes to determine target cells/nuclei and non-target cells/nuclei.

66. The method of any one of claims 64-65, wherein before step (c), wherein the method further comprises amplifying the first set of amplicon products with adapter sequences to produce a second set of amplicon products.

67. The method of any one of claims 64-66, wherein the method further comprises, after step (b) or (c), contacting the first set of amplicon products with sample barcoding sequences.

68. The method of any one of claims 64-67, wherein said sample barcoding sequences comprise a set of forward and/or reverse barcoding primers, and wherein the method comprises amplifying the first set of amplicon products with the set of forward and/or reverse barcoding primers to produce a barcoded indexed library comprising barcoded amplicon products.

69. The method of any one of claims 64-68, wherein said barcoding sequences comprise a set of forward and/or reverse barcoding adapters, and wherein the method comprises ligating the set of forward and/or reverse barcode adapters to produce a barcoded indexed library comprising barcoded amplicon products.

70. The method of any one of claims 64-69, wherein before step (b), the method comprises fixing and/or permeabilizing the/nuclei population.

71. The method of any one of claims 64-70, wherein the primer pool set comprises primers that hybridize to a target region of a target sequence of the DNA within the/nuclei population.

72. The method of any one of claims 64-71, wherein the primer pool set further comprises indexing primers.

73. The method of any one of claims 64-72, wherein the sample is a cell suspension generated from a tissue sample or a cell suspension generated from a liquid biopsy.

74. The method of any one of claims 64-73, wherein the sample is a Formalin-Fixed Paraffin-Embedded (FFPE) tissue sample or a cryopreserved tissue sample.

75. The method of any one of claims 64-74, wherein said sequencing comprises next generation sequencing.

76. The method of any one of claims 64-75, wherein the method further comprises, after step (b), sorting the cell/nucleic population into subpopulations by phenotypes to determine target cells/nucleic and non-target cells/nuclei.

77. The method of any one of claims 64-76, wherein said contacting occurs after lysing in step (c).

78. The method of any one of claims 64-77, wherein the cell population comprises target cells having 3-10 cells.

79. The method of any one of claims 64-78, where multiple PCR reactions are performed between steps (b) and (e).

80. The method of any one of claims 64-79, wherein before step (b), the method comprises fixing and/or permeabilizing the heterogenous cell population.

81. A kit for amplicon-based library preparation in situ, the kit comprising:
a cell preservation agent capable of preserving a cell/nuclei population, the cell preservation agent selected from: a fixative, a permeabilizer, or a fixative and a permeabilizer;
a primer pool set capable of amplifying a target sequence region of DNA within one or more cells of the cell/nuclei population;
a polymerase chain reaction (PCR) Enzyme Master Mix comprising one or more of: an enzyme, a buffer, or an enzyme and a buffer;
a cell lysis buffer;
in an amount sufficient to prepare an amplicon-based library in situ for sequencing; and
instructions for carrying out the amplicon-based library preparation in situ, the instructions providing the following steps:
amplifying the target sequence region of DNA in the cell/nuclei population to produce a first set of amplicon products for each cell;
lysing each of the cells to isolate DNA fragments having the target sequence region within the first set of amplicon products;
purifying the DNA fragments; and
sequencing the DNA fragments.

82. The kit of claim 81, wherein the kit further comprises protease K for the lysing step.

83. The kit of any one of claims 81-82, wherein the kit further comprises barcoding primers, and a second PCR Enzyme master mix comprising one or more of: an enzyme, a buffer, or an enzyme and a buffer.

84. A kit for ligation-based library preparation in situ, the kit comprising:
a cell preservation agent capable of preserving a cell/nuclei population, the cell preservation agent selected from: a fixative, a permeabilizer, or a fixative and a permeabilizer;
a fragmentation enzyme and buffer for performing an enzymatic fragmentation reaction to form DNA fragments within the cell/nuclei population;
an End repair and A tail (ERA) master mix and buffer for performing an end-repair and A-tailing reaction on the one or more DNA fragments;
a ligation enzyme and buffer;
adapter sequences, wherein the ligation enzyme and buffer, and adapter sequences are capable of ligating, in each cell, the DNA fragments to the adapter sequences in situ to create a ligated library comprising ligated DNA fragments;
amplification primers for amplifying the ligated DNA fragments to form amplicon products;
a polymerase chain reaction (PCR) enzyme master mix comprising one or more of: an enzyme, a buffer, or an enzyme and a buffer;
a cell lysis buffer;
in an amount sufficient to prepare a ligation-based library in situ for sequencing; and
instructions for carrying out the ligation-based library preparation in situ, the instructions providing the following steps:
performing, in each cell of the cell/nuclei population, an enzymatic fragmentation reaction to form DNA fragments within the cell/nuclei population;
ligating, in each cell, the DNA fragments to adapter sequences in situ to create the ligated library comprising ligated DNA fragments;
lysing each of the cells to collect the ligated DNA fragments;
purifying the ligated DNA fragments; and
sequencing the ligated DNA fragments.

85. The kit of claim 84, wherein the amplification primers comprise barcoding primers, sequencing primers, or a combination thereof.

86. The kit of any one of claims 84-85, wherein the kit further comprises protease K for the lysing step.

87. The kit of any one of claims 84-86, wherein the kit further comprises barcoding primers, and a second PCR Enzyme master mix comprising one or more of: an enzyme, a buffer, or an enzyme and a buffer.
